(19) United States
(12) Patent Application Publication
(10) Pub. No.: US 2023/0184766 A1
(43) Pub. Date: Jun. 15, 2023

(54) COMPOSITIONS AND METHODS FOR CORONAVIRUS DETECTION

(71) Applicant: The Johns Hopkins University, Baltimore, MD (US)

(72) Inventors: Robert Layne Kruse, Baltimore, MD (US); Yuting Huang, Baltimore, MD (US); Zack Z. Wang, Baltimore, MD (US)

(21) Appl. No.: 17/922,662

(22) PCT Filed: Apr. 30, 2021
(86) PCT No.: PCT/US2021/030212

§ 371 Date: Nov. 1, 2022

Related U.S. Application Data
(60) Provisional application No. 63/019,022, filed on May 1, 2020.
(60) Provisional application No. 63/070,463, filed on Aug. 26, 2020.

(51) Int. Cl. G01N 33/569; C07K 14/005; C07K 16/34

(52) U.S. Cl. CPC G01N 33/56983; C07K 14/005; C07K 16/34; G01N 2333/165; G01N 2469/20; G01N 2469/10; C12N 2770/20022; C07K 2319/33; C07K 2317/76; C07K 2317/622; C07K 2317/569; C07K 2317/522

(57) ABSTRACT

Disclosed herein, are recombinant polypeptides comprising a first domain, wherein the first domain comprises an epitope of a coronavirus or wherein the first domain is a moiety that is capable of specifically binding a coronavirus antigen; a linker; and a second domain, wherein the second domain is a moiety that is capable of specifically binding an antigen on the surface of a red blood cell. Also, disclosed herein are methods for detecting anti-coronavirus antibodies, one or more coronavirus antigens, or one or more coronavirus virions by mixing the recombinant polypeptide with red blood cells and anti-coronavirus antibodies resulting in visible agglutination.

### CROSS REFERENCE TO RELATED APPLICATIONS

This application claims the benefit of U.S. Provisional Application No. 63/019,022, filed on May 1, 2020, and 63/070,463, filed on Aug. 26, 2020. The content of these earlier filed applications is hereby incorporated by reference herein in their entirety.

### INCORPORATION OF THE SEQUENCE LISTING

The present application contains a sequence listing that is submitted via EFS-Web concurrent with the filing of this application, containing the file name “36406_0020P1_SL.txt” which is 139,264 bytes in size, created on Apr. 28, 2021, and is herein incorporated by reference in its entirety.

### FIELD OF THE INVENTION

The present invention relates to compositions and methods for detecting antibodies against severe acute respiratory syndrome coronavirus 2 antigens, and more particularly to methods that are a rapid, point of care red blood cell (RBC) agglutination test based on fusion proteins of viral antigens and single chain variable fragments against RBC antigens. The present invention also describes compositions for detecting severe acute respiratory syndrome coronavirus 2 antigens using recombinant polypeptides (e.g., bispecific antibody fusion proteins). These methods can be useful in detecting agglutination upon the addition of a fusion protein to COVID-19 whole blood within two minutes.

### BACKGROUND

An urgent need exists for a scalable solution to identify antibodies in recovered COVID-19 patients, which can identify subjects infected with virus previously and suggest who might have immune protection. An urgent need also exists for detection of COVID-19 infection in patients, which can be performed by testing for the presence of SARS-CoV-2 antigens in the bloodstream of patients. Given the global scale of the pandemic, a solution should be low-cost and function without the need for machines.

### SUMMARY

The COVID-19 pandemic has brought the world to a halt, with cases observed around the globe causing significant mortality. An urgent need exists for serological tests to detect antibodies against SARS-CoV-2, which could provide information related to the extent of infection in populations, as well as ascertain whether individuals may be protected from future infection. Current serological tests developed for SARS-CoV-2 rely on traditional technologies such as enzyme-linked immunosorbent assays and lateral flow assays, which may lack scalability to meet the demand of hundreds of millions of antibody tests in the coming year. Described herein, are methods of antibody testing that uses one protein reagent that is added to patient serum or whole blood. The assay tags the red blood cell (RBC) surface with a fusion protein of SARS-CoV-2 antigen (e.g., spike domains or nucleocapsid) connected to a single-chain variable fragment (scFv) against an RBC antigen, such as the carbohydrate H antigen or Glycophorin A. The results show that upon mixing of the fusion protein with recovered COVID-19 patient serum and RBCs, agglutination of RBCs is observed, indicating that the patient developed antibodies against SARS-CoV-2. Given that the test uses methods that can be used in hospital clinical labs across the world, the test can be rapidly deployed with the protein reagent requiring manufacturing for a projected cost of U.S. cents per test. The assay described herein can also be uses in low-resource settings for detecting SARS-CoV-2 antibodies.

The SARS-CoV-2 virus causing COVID-19 disease represents a growing pandemic infection around the world. SARS-CoV-2 is a coronavirus infecting cells primarily in the lungs and gastrointestinal tract, leading to acute respiratory distress syndrome in a small portion of patients and ultimately significant mortality (Guan, W.-J. et al. N. Engl. J. Med. NEJMoa2002032 (2020)). SARS-CoV-2 imposes both diagnostic and therapeutic challenges. On the diagnostic front, reverse transcriptase-polymerase chain reaction (RT-PCR) is the gold standard for detecting the virus, but PCR represents significant costs in low-income countries and is not always widely available (Tang, Y.-W., et al. J. Clin. Microbiol. (2020)). Importantly, RT-PCR cannot detect evidence of past infection, which will be important for epidemiological efforts to assess how many people have been infected. While viral load determined by RT-PCR appears to have prognostic value (Liu, Y. et al. Lancet Infect Dis (2020)), measuring the immune response against SARS-CoV-2 may also be beneficial into assessing the outcomes of patients (Wolfel, R. et al. Nature 1-10 (2020)).

Serologic testing for antibodies against SARS-CoV-2 could detect and diagnose both current and past infection, which is important for surveillance and epidemiological studies (Guo, L. et al. Clin Infect Dis. 24, 490 (2020)). No serologic testing platforms exist widely for COVID-19, however. An urgent need exists for a low complexity assay that could be performed as a point of care test in low-income countries, without the need for machines. However, current enzyme-linked immunosorbent assay (ELISA) tests for COVID-19 require a number of steps, washes, and reagents, involving hours of manual time and/or automated machines (Okba, N. M. A. et al. Emerging Infect. Dis. 26, 270 (2020)). Lateral flow immunoassays have been developed for SARS-CoV-2, but still require the manufacturing of strips, plastic holders, and multiple different antibody types and conjugates (Li, Z. et al. J. Med. Virol. jmv.25727 (2020)). A rapid lateral flow immunoassay is also limited as one-time use.

As an alternative method to detect antibodies, blood banks across the world routinely detect antibodies against blood group antigens as part of the type and screen assay, necessary before blood transfusions can be given. The readout of the assay is hemagglutination, or the aggregation of red blood cells (RBCs), which can be captured by a camera or easily observed with the naked eye. Furthermore, hemagglutination testing, whether by hand or by an automated machine, can be used to titer antibodies, measuring their levels in the serum (Lally, K. et al. Transfusion 60, 628-636 (2020)). This particular flexibility to range from point of care, single patient testing to wide scalable on existing automated platforms in clinical labs is uncommon among the different serologic technology options.

Hemagglutination has been leveraged in the past to detect antibodies against pathogens. The first iteration consisted of cross-linking an antibody against RBC antigens with a peptide antigen from human immunodeficiency virus (HIV) (Kemp, B. E. et al. Science 241, 1352-1354 (1988); and Wilson, K. M., et al. J Immunol. Methods 175, 267-273 (1994)). When incubated with whole blood from HIV patients, RBC agglutination could be observed, indicating antibodies specific to that antigen were detected (Kemp, B. E. et al. Science 241, 1352-1354 (1988)). A comparison of 1800 patient blood specimens found similar sensitivity and specificity among commercial ELISA kits and 2-minute autologous RBC agglutination testing (Sirivichayakul, S. et al. J. Clin. Microbiol. 31, 1373-1375 (1993)). Later studies improved on the technology by building fusion proteins of antibody fragments with antigens from HIV (Gupta, A., et al. J. Immunol. Methods 256, 121-140 (2001); and Gupta, A. & Chaudhary, V. K. Protein Expr. Purif 26, 162-170 (2002)). Targeting multiple different RBC antigens at the same time improved performance characteristics of the assay (Gupta, A. & Chaudhary, V. K. J. Clin. Microbiol. 41, 2814-2821 (2003)). Antibodies against West Nile virus have also been to be detectable by autologous RBC agglutination assay (Hobson-Peters, J., et al. Journal of Virological Methods 168, 177-190 (2010)). Outside of infectious disease, elevated D-dimer levels could also be detected with a similar red agglutination assay, SimpliRED, for point of care testing for patients with suspected deep vein thrombosis (John, M. A. et al. Thromb. Res. 58, 273-281 (1990); and Wells, P. S. et al. Lancet 351, 1405-1406 (1998)).

Described herein are methods that use RBC agglutination to detect antibodies against SARS-CoV-2 spike protein in COVID-19 patients. This method can be used in low-resource settings as a rapid method of testing for current or past SARS-CoV-2 infection.

An urgent need exists for a scalable solution to identify antibodies in recovered COVID-19 patients, which can identify subjects infected with the coronavirus previously and suggest who might have immune protection. Given the global scale of the pandemic, the methods should be low-cost and function without the new for machines. Described herein is a rapid, point of care red blood cell (RBC) agglutination test based on fusion proteins of viral antigens and single chain variable fragments against RBC antigens. In some aspects, upon the addition of fusion protein to COVID19 whole blood, agglutination can be detected within two minutes after mixing one drop of blood, for example, on a card. In some aspects, the fusion protein can already be dried onto the card, such that just the drop of blood needs to be added to the card, followed by solubilization of the fusion protein and the agglutination reaction to occur by mixing.

Disclosed herein are recombinant polypeptides comprising: a first domain, wherein the first domain comprises an epitope of a coronavirus; a linker; and a second domain, wherein the second domain is a moiety that is capable of specifically binding an antigen on the surface of a red blood cell.

Disclosed herein are recombinant polypeptides comprising: a first domain, wherein the first domain is a moiety that is capable of specifically binding a coronavirus antigen; a linker; and a second domain, wherein the second domain is a moiety that is capable of specifically binding an antigen on the surface of a red blood cell.

### BRIEF DESCRIPTION OF THE DRAWINGS

FIGS. 1A-B show the construction of a fusion protein and the mechanism of agglutination. FIG. 1A shows a fusion protein consisting of the receptor-binding domain (RBD) of the SARS-CoV-2 spike protein connected via a linker to a single chain variable fragment (scFv, consisting of VH and VL domains connected with a flexible linker) targeting the H antigen on the surface of the red blood cells (RBCs). Antibodies targeting the RBD could potentially crosslink two different RBCs. FIG. 1B shows the cross-linking of multiple RBCs in mass would eventually lead to visible agglutination being seen with the naked eye. VH=variable heavy; VL=variable light.

FIGS. 2A-B shows RBD-scFv fusion protein can be successfully isolated. FIG. 2A shows the size of RBD-2E8 protein expressed in 293T cells was confirmed on protein gel electrophoresis demonstrating expected band size (˜57 kDa). A cartoon image of RBD-2E8 is also provided, along with sequence information. SEQ ID NO: 38 is shown. FIG. 2B shows the size of B6-CH1-RBD expressed in E. coli is confirmed on protein gel electrophoresis demonstrating expected band size (˜66 kDa). A cartoon image of B6-CH1-RBD is also provided, along with sequence information. His-tag sequence for purification of fusion proteins is also included. SEQ ID NO: 39 is shown.

FIGS. 3A-C shows RBD-scFv (e.g., RBD-2E8) mediates agglutination of the red blood cells in the presence of COVID-19 patient serum after 5 min. FIG. 3A shows the control agglutination reaction was performed for 5-min between B and O anti-sera and A cells. FIG. 3B shows bacterial B6-CH1-RBD and FIG. 3C shows mammalian RBD-2E8 proteins were serially diluted in the presence of fixed concentrations of RBC and undiluted COVID-19 convalescent serum from a single patient. Agglutination was seen in the highest three concentrations of RBD-2E8 after 5-min of incubation, most intense in the highest concentration of RBD-2E8. Negative control contained phosphate-buffered solution (PBS) in the place of patient serum. Bacterial B6-CH1-RBD showed no agglutination at any protein concentration.

FIGS. 4A-D show prolonged hemagglutination assay yields strong agglutination. A longer, 1 h incubation was performed, whereafter the plate was tilted; downward motion of RBCs indicates no agglutination, while spreading RBC surface or stable RBC pellet indicates agglutination. FIG. 4A shows a control agglutination reaction was performed between B and O anti-sera and A cells. FIG. 4B shows mammalian RBD-2E8 and B6-CH1-RBD were mixed with ACE2-Fc (500 ng), CR3022 (30 ng), or PBS and evaluated at 1 h. FIG. 4C shows that to test the analytic sensitivity, a dilution series of CR3022 antibody (ng) was prepared and tested to detect the lowest antibody concentration yielding agglutination. FIG. 4D shows that three different COVID-19 patient sera were incubated with B6-CH1-RBD, RBD-2E8, and RBCs. Control conditions had PBS alone and non-infected patient plasma.

FIGS. 5A-D show the components and steps of the methods disclosed herein. FIG. 5A shows a schematic of the recombinant polypeptide that can be used to detect coronaviral antigen. FIG. 5B shows that the recombinant polypeptide is bispecific and allows for decorating the surface of red blood cells with a capture antibody against the coronavirus antigen. FIG. 5C shows that in the presence of SARS-CoV-2 virions would trigger binding of different red blood cells in the presence of bispecific antibodies. FIG. 5D shows that in the presence of SARS-CoV-2 nucleocapsid would trigger binding of different red blood cells in the presence of bispecific antibodies.

FIGS. 6A-B show the Eldon Card as a test of point of care, hemagglutination for ABO typing. The Eldon Card (FIG. 6A) is a commercially sold point of care test for blood typing. The kit (FIG. 6B) includes an alcohol pad to sterilize a fingertip, and lancet to yield drops of blood. Plastic sticks are included to help collect and stir blood drops. The card is kept in a desiccating pouch for shipping and is stable at room temperature. Results from testing an A-positive person is shown, demonstrating strong agglutination.

FIG. 7 shows the mechanism of a dry card assay for hemagglutination-based detection of SARS-CoV-2 antibodies. A fusion protein consisting of a nanobody targeting human glycophorin A and the receptor binding domain (RBD) of SARS-CoV-2 was used. The fusion proteins are dried onto a non-water absorbent card, remaining stable at room temperature indefinitely in a desiccant pouch. For testing, the fusion proteins are resuspended in a water droplet, followed by the addition of whole blood containing antibodies and RBCs. Stirring facilitates cross-linking of large aggregates of RBCs, which are visible by the naked eye.

FIG. 8 shows that hemagglutination can be scored for reaction strength. Scores were developed to quantify the degree of agglutination observed across COVID-19 convalescent samples. Cards are depicted horizontal on table surface after testing. Strong agglutinations (ex: 4) quantified the majority of red blood cells sticking together, with a white background without unbound cells. Weaker reactions (ex: 2) had smaller, but frequent agglutinations. The scores of 0 and 1 were deemed negative for the purpose of the test.

FIG. 9 shows that the tilted cards can facilitate agglutination scoring. Tilted cards were evaluated for agglutination after the last step stirring step. Strong agglutinations (ex: 4) quantified the majority of red blood cells sticking together, with a white background without unbound cells. Weaker reactions (ex: 2) had smaller, but frequent agglutinations, that were observed during card tilting. The scores of 0 and 1 were deemed negative for the purpose of the test.

FIGS. 10A-B show that COVID-19 recovered patients exhibit a distribution of agglutination scores that correlate with the dilution of COVID-19 convalescent serum. FIG. 10A shows the agglutination scores for 200 patient samples were tabulated, and percentages for each agglutination score provided. Agglutination scores of 1 and 0 were deemed negative and are stripped for distinction from the positive test results in solid color. FIG. 10B show a sample with a strong agglutination (4) was obtained, and the serum progressively diluted with pre-pandemic serum. The same amount of RBC's was added to all conditions. Agglutinations are depicted in the titled position, and were clearly seen down to 1:10, while 1:50 only had very weak agglutinations below the assay cutoff.

FIGS. 11A-C show that agglutination scores correlate with ELISA and neutralizing antibody titer assays. FIG. 11A shows the optical density (OD) of the Euroimmun Spike IgG ELISA (1:100) that was categorized for each agglutination score. FIG. 11B shows neutralizing antibody AUC (area under curve) that was quantified for each specimen, and plotted against the respective agglutination score. FIG. 11C shows a scatter plot of neutralizing antibody titers at different agglutination score is presented. Each dot represents a single sample, and each bar represents the median among the samples. Statistical analysis was performed using parametric, t-tests for ELISA and AUC data, and a non-parametric, Mann-Whitney U test for titer data. Statistical significance (* p<0.05, ** p<0.01 *** p<0.001).

FIGS. 12A-C show that specimens requiring longer assay time to yield agglutination yield low agglutination scores and neutralizing antibody titer. A subset of convalescent COVID-19 samples (n=73) was scored after the first round of card tilting, followed by three minutes incubation and a second round of card tilting for a second score. FIG. 12A shows that the distribution of samples that were positive already at the first round (+/+), positive after the second round (−/+), and negative after the complete assay (−/−). FIG. 12B shows the agglutination scores of −/+ samples, demonstrating most scored a 2 agglutination score. FIG. 12C shows the neutralizing antibody titers for negative (−/−), delayed agglutination (−/+), and fast (+/+) samples with low agglutination scores, 2 and 2.5, respectively. The latter corresponds to the ultimate agglutination scores observed by −/+ samples. Each point represents a single sample, and each bar represents the median of the group. Statistical analysis was performed using a non-parametric, Mann-Whitney U test. Statistical significance (* p<0.05) n.s.=non=significant.

FIGS. 13A-C show that false positive samples on hemagglutination test yield weak agglutinations, while high-titer false negative agglutinations are rare. Nine samples that were false positives among the pre-pandemic, emergency department samples were further analyzed. FIG. 13A shows the distribution of samples that were positive already at the first round (+/+) versus positive after the second round (−/+). FIG. 13B shows the distribution of agglutination scores among false positive samples are also demonstrated, generally showing very weak agglutination. FIG. 13C show neutralizing antibody titers for false negative samples among the hemagglutination-based test, Euroimmun Spike IgG ELISA, and the CoronaChek lateral flow assay. Each point represents a single sample, and each bar represents the median of the group. Statistical analysis was performed using a non-parametric, Mann-Whitney U test. n.s.=non=significant.

FIG. 14 shows the relationship between Spike IgG ELISA and neutralizing antibody levels. The optical density (OD) for the Euroimmun Spike IgG ELISA at 1:100 dilution is characterized versus the neutralizing antibody titer AUC (area under curve) for each COVID-19 convalescent plasma specimen.

### DETAILED DESCRIPTION

The present disclosure can be understood more readily by reference to the following detailed description of the invention, the figures and the examples included herein.

Before the present methods and compositions are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### Definitions

As used in the specification and the appended claims, the singular forms “a,” “an” and “the” include plural referents unless the context clearly dictates otherwise.

The word “or” as used herein means any one member of a particular list and also includes any combination of members of that list.

Ranges can be expressed herein as from “about” or “approximately” one particular value, and/or to “about” or “approximately” another particular value. When such a range is expressed, a further aspect includes from the one particular value and/or to the other particular value. Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “sample” is meant a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

As used herein, the term “subject” refers to the target of administration, e.g., a human. Thus the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In some aspects, a subject is a human. The term does not denote a particular age or sex. Thus, adult, child, adolescent and newborn subjects, as well as fetuses, whether male or female, are intended to be covered.

As used herein, the term “patient” refers to a subject afflicted with a disease or disorder. The term “patient” includes human and veterinary subjects. In some aspects of the disclosed methods, the “patient” has been identified with a need for testing for suspected coronavirus exposure, such as, for example, prior to a blood draw.

As used herein, the term “comprising” can include the aspects “consisting of” and “consisting essentially of.”

A “SARS virus protein” refers to any protein of any SARS virus strain or its functional equivalent as defined herein. Thus, the invention includes, but is not limited to, SARS polymerase, the S (spike) protein, the N (nucleocapsid) protein, the M (membrane) protein, the small envelope E protein and their functional equivalents.

“Epitope” as used herein refers to an antigenic determinant of a polypeptide. An epitope could comprise three amino acids in a spatial conformation which is unique to the epitope. Generally, an epitope consists of at least five such amino acids, and more usually consists of at least 8-10 such amino acids. Methods of determining the spatial conformation of such amino acids are known in the art.

As used herein, the term “polypeptide” refers to any peptide, oligopeptide, polypeptide, gene product, expression product, or protein. A polypeptide is comprised of consecutive amino acids. The term “polypeptide” encompasses naturally occurring or synthetic molecules. As used herein, the term “amino acid sequence” refers to a list of abbreviations, letters, characters or words representing amino acid residues. Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

By “isolated polypeptide” or “purified polypeptide” is meant a polypeptide (or a fragment thereof) that is substantially free from the materials with which the polypeptide is normally associated in nature. The polypeptides of the invention, or fragments thereof, can be obtained, for example, by extraction from a natural source (for example, a mammalian cell), by expression of a recombinant nucleic acid encoding the polypeptide (for example, in a cell or in a cell-free translation system), or by chemically synthesizing the polypeptide. In addition, polypeptide fragments may be obtained by any of these methods, or by cleaving full length polypeptides.

By “specifically binds” is meant that an antibody recognizes and physically interacts with its cognate antigen (for example, a c-Met polypeptide) and does not significantly recognize and interact with other antigens; such an antibody may be a polyclonal antibody or a monoclonal antibody, which are generated by techniques that are well known in the art.

As used herein, the term “neutralize” refers to the ability of an antibody, or antigen binding fragment thereof, to bind to an infectious agent, such as coronavirus, and reduce the biological activity, for example, virulence, of the infectious agent. An antibody can neutralize the activity of an infectious agent, at various points during the lifecycle of the virus. For example, an antibody may interfere with viral attachment to a target cell by interfering with the interaction of the virus and one or more cell surface receptors. Alternately, an antibody may interfere with one or more post-attachment interactions of the virus with its receptors, for example, by interfering with viral internalization by receptor-mediated endocytosis.

As used herein, the terms “recombinant polypeptide” or “fusion protein” refers to a composition comprising a first domain comprising an epitope of a coronavirus; a linker; and a second domain, wherein the second domain is a moiety that is capable of specifically binding an antigen on the surface of a red blood cell. In some aspects, the first domain can comprise two or more epitopes of one or more coronaviruses.

As used herein, the term “determining” can refer to measuring or ascertaining an activity or an event or a quantity or an amount or a change in expression and/or in activity level or in prevalence and/or incidence. For example, determining can refer to measuring or ascertaining the quantity or amount of red blood cell agglutination. Methods and techniques used to determining an activity or an event or a quantity or an amount or a change in expression and/or in activity level or in prevalence and/or incidence as used herein can refer to the steps that the skilled person would take to measure or ascertain some quantifiable value. The art is familiar with the ways to measure an activity or an event or a quantity or an amount or a change in expression and/or in activity level or in prevalence and/or incidence.

Compositions

Recombinant polypeptides. As used herein, the term “recombinant polypeptide” refers to a polypeptide generated by a variety of methods including recombinant techniques. In some aspects, the recombinant polypeptide comprises a first domain comprising an epitope of a coronavirus; a linker; and a second domain, wherein the second domain is a moiety that is capable of specifically binding an antigen on the surface of a red blood cell. In some aspects, the first domain can be a moiety that is capable of specifically binding a coronavirus antigen. Tables 1 and 2 list polypeptide sequences.

In some aspects, the first domain can be derived from a viral antigen. In some aspects, a domain of the viral antigen can be used. In some aspects, an epitope of the viral antigen can be used. In some aspects, the first domain comprises a sequence that does not dimerize or multimerize in a solution. In some aspects, the first domain comprises a sequence that remains a monomer in solution. In some aspects, the first domain can comprise one or more epitopes or one or more viral antigens. In some aspects, two or more epitopes can be from the same or different coronavirus. In some aspects, two or more viral antigens can be from the same or different proteins from the same or different coronaviruses.

In some aspects, the first domain can comprise a sequence from a coronavirus spike (S) protein, envelope (E) protein, a membrane (M) protein, a nucleocapsid (N) protein or an antigenic fragment thereof. In some aspects, the first domain can comprise a sequence from a coronavirus spike (S) protein, envelope (E) protein, a membrane (M) protein, a nucleocapsid (N) protein, an antigenic fragment thereof or a combination thereof. In some aspects, the S protein sequence can comprise a S1 domain, a S2 domain, the N-terminal domain, a receptor-binding domain or the entire S protein ectodomain. In some aspects, the antigenic fragment thereof can comprise an immunodominant epitope from coronavirus spike (S) protein, envelope (E) protein, a membrane (M) protein, or nucleocapsid (N) protein.

In some aspects, the first domain can comprise one or more of SEQ ID NOs: 5-15 or an antigenic fragment thereof.

In some aspects, wherein the second domain can be a single chain variable fragment (scFv), a Fab, a camelid antibody, a nanobody, a shark vNAR antibody, or a peptide. In some aspects, the peptide can be adnectins, anticalins, avimers, Fynomers, Kunitz domains, knottins, affibodies, p-hairpin mimetics. In some aspects, the peptide can be an ankyrin repeat protein. In some aspects, the peptide can be 10 to 100 amino acids long. In some aspects, the peptide is capable of binding to an antigen on the surface of a red blood cells. In some aspects, the peptide can be derived from an erythrocyte-binding sequence. In some aspects, the erythrocyte-binding sequence can be from one or more Plasmodium proteins. Examples of erythrocyte-binding sequences include but are not limited to Plasmodium proteins, wherein the Plasmodium protein can be serine repeat antigen (SERA), STEVOR, erythrocyte binding antigen-175, erythrocyte binding antigen-181, erythrocyte binding antigen-140, erythrocyte-binding ligand-1, or P falciparum glutamic acid-rich protein (PfGARP). The following publications are incorporated herein by reference to disclose and describe the sequences in connection with examples of erythrocyte-binding sequences: Almukadi et al., Blood. 2019 Jan. 31; 133(5):470-480; Puentes et al., Parasitol Int. 2000 August; 49(2):105-17; Garcia et al., Peptides. 2005 July; 26(7):1133-43; Jakobsen et al., Infect Immun. 1998 September; 66(9):4203-7; and Jaskiewicz et al., Parasites & Vectors volume 12, Article number: 317 (2019).

In some aspects, the scFv can be constructed from the variable domains of the heavy (VH) and light (VL) chain, wherein the C-terminus of the VH can be linked to the N-terminus of the VL using a flexible linker. Examples of flexible linkers are described herein.

In some aspects, the second domain can be a sequence that is capable of specifically binding an antigen on the surface of a red blood cell. In some aspects, the sequence specific for an antigen on the surface of a red blood cell can be an antibody or a fragment thereof.

In some aspects, the second domain can be a moiety that is capable of specifically binding an antigen on the surface of a red blood cell. In some aspects, the antigen on the surface of the red blood cell can be a carbohydrate antigen. In some aspects, the carbohydrate antigen can be a H antigen, a antigen, a B antigen, a I antigen, or a Lewis antigen. In some aspects, the antigen on the surface of the red blood cell can be a protein antigen. In some aspects, the protein antigen can be a Rh antigen, a Kell antigen, a Kidd antigen, a Duffy antigen, a Lutheran antigen, a glycophorin A, or a glycophorin B. The human sequences of carbohydrate and protein antigens disclosed herein are known. The following publications are incorporated herein by reference to disclose and describe the sequences in connection with examples of D antibody sequence, antibodies against ABO and I blood group systems (B and HI), of the Rh system (D and E) and of the Kell system (Kpb), and Lutheran antibody sequence: Dziegiel et al., J Immunol Methods. 1995 May 11; 182(1):7-19; Proulx et al., Transfusion. 2002 January; 42(1):59-65; Marks et al., Bio/Technology volume 11, pages 1145-1149(1993); Richard et al., Transfusion. 2006 June; 46(6):1011-7.

In some aspects, the scFv can be derived from an antibody that is capable of specifically binding to an antigen on the surface of a red blood cell. In some aspects, the scFv can be derived from an antibody that is capable of specifically binding to the surface of a red blood cell. In some aspects, the scFv can be derived from an antibody that is capable of specifically binding to red blood cell surface antigens. In some aspects, the scFv can be 10F7, A41, B6, 2E8, 1C3, ABO.B1, ABO.HI1, Rh.D1, Rh.E1, Ery.X1, K.Kpb1, 4G11, or a single domain antibody IH4.

In some aspects, the second domain can comprise one of SEQ ID NOs: 1-4.

TABLE 1
Examples of polypeptide sequences.

| SEQ ID | | |
|---|---|---|
| NO: | Sequence | Name |
| 1 | QVQLKESGPGLVAPSSQLSITCTVSGFSLSGYSVHWVRQPPGK | 2E8 scFv |
| | GLEWLGMIWGGGNTDYKSALKSRLTISKDNSRSQVLLKMNSL | |
| | QIDDTAIYYCARNYGYSPFVHWGQGTLVTVSAGGGGSGGGGS | |
| | GGGGSDIVMTQSPSSLAMSVGQKVTMSCKSSQSLLNSDNQKN | |
| | YLAWYQQKPGQSPKLLVYFASSRESGVSDRFIGSGSGTDFTLTI | |
| | GSVQSEDLAYYFCQQLYRTPFTFGSGTKLEIK | |
| 2 | QVKLVQSGPELVKPGASVKMSCKASGYTFTNYVMHWVKQQP | B6 scFv |
| | GQVLEWIGYINPYNDDTKYNEKFKGKATLASDKSSNTAYMEL | |
| | SSLPSEDSAVYYCASGGYYTMDYWGQGTSVTVSSGGGGSGG | |
| | GGSGGGGSDIVLTQSPLSLPVSLGDQASISCRSSQSLVHSKGYT | |
| | YLHWYLQKPGQSPKLLIYKVSRRFSGVPDRFNGSGSGTDFTLK | |
| | ISRVEAEDLGVYFCSQSTHVPYTFGGGTKLELKRA | |
| 3 | QVKLQQSGAELVKPGASVKLSCKASGYTFNSYFMHWMKQRP | 10F7 scFv |
| | VQGLEWIGMIRPNGGTTDYNEKFKNKATLTVDKSSNTAYMQL | |
| | NSLTSGDSAVYYCARWEGSYYALDYWGQGTTVTVSSGGGGS | |
| | GGGGSSGGGGSSDIELTQSPAIMSATLGEKVTMTCRASSNVKY | |
| | MYWYQQKSGASPKLWIYYTSNLASGVPGRFSGSGSGTSYSLTI | |
| | SSVEAEDAATYYCQQFTSSPYTFGGGTKLEIKRAAA | |
| 4 | QVQLQESGGGSVQAGGSLRLSCVASGYTDSTYCVGWFRQAPG | IH4 VHH |
| | KEREGVARINTISGRPWYADSVKGRFTISQDNSKNTVFLQMNS | nanobody |
| | LKPEDTAIYYCTLTTANSRGFCSGGYNYKGQGQVTVS | |
| 5 | VSSQCVNLTTRTQLPPAYTNSFTRGVYYPDKVFRSSVLHSTQD | SARS-CoV- |
| | LFLPFFSNVTWFHAIHVSGTNGTKRFDNPVLPFNDGVYFASTE | 2 Spike |
| | KSNIIRGWIFGTTLDSKTQSLLIVNNATNVVIKVCEFQFCNDPFL | protein, |
| | GVYYHKNNKSWMESEFRVYSSANNCTFEYVSQPFLMDLEGK | extracellular |
| | QGNFKNLREFVFKNIDGYFKIYSKHTPINLVRDLPQGFSALEPL | domain |
| | VDLPIGINITRFQTLLALHRSYLTPGDSSSGWTAGAAAYYVGY | |
| | LQPRTFLLKYNENGTITDAVDCALDPLSETKCTLKSFTVEKGIY | |
| | QTSNFRVQPTESIVRFPNITNLCPFGEVFNATRFASVYAWNRKR | |
| | ISNCVADYSVLYNSASFSTFKCYGVSPTKLNDLCFTNVYADSF | |
| | VIRGDEVRQIAPGQTGKIADYNYKLPDDFTGCVIAWNSNNLDS | |
| | KVGGNYNYLYRLFRKSNLKPFERDISTEIYQAGSTPCNGVEGF | |
| | NCYFPLQSYGFQPTNGVGYQPYRVVVLSFELLHAPATVCGPK | |
| | KSTNLVKNKCVNFNFNGLTGTGVLTESNKKFLPFQQFGRDIAD | |
| | TTDAVRDPQTLEILDITPCSFGGVSVITPGTNTSNQVAVLYQDV | |
| | NCTEVPVAIHADQLTPTWRVYSTGSNVFQTRAGCLIGAEHVN | |
| | NSYECDIPIGAGICASYQTQTNSPRRARSVASQSIIAYTMSLGAE | |
| | NSVAYSNNSIAIPTNFTISVTTEILPVSMTKTSVDCTMYICGDST | |
| | ECSNLLLQYGSFCTQLNRALTGIAVEQDKNTQEVFAQVKQIYK | |
| | TPPIKDFGGFNFSQILPDPSKPSKRSFIEDLLFNKVTLADAGFI | |
| | KQYGDCLGDIAARDLICAQKFNGLTVLPPLLTDEMIAQYTSALLA | |
| | GTITSGWTFGAGAALQIPFAMQMAYRFNGIGVTQNVLYENQK | |
| | LIANQFNSAIGKIQDSLSSTASALGKLQDVVNQNAQALNTLVK | |
| | QLSSNFGAISSVLNDILSRLDKVEAEVQIDRLITGRLQSLQTYVT | |
| | QQLIRAAEIRASANLAATKMSECVLGQSKRVDFCGKGYHLMS | |
| | FPQSAPHGVVFLHVTYVPAQEKNFTTAPAICHDGKAHFPREGV | |
| | FVSNGTHWFVTQRNFYEPQIITTDNTFVSGNCDVVIGIVNNTV | |
| | YDPLQPELDSFKEELDKYFKNHTSPDVDLGDISGINASVVNIQK | |
| | EIDRLNEVAKNLNESLIDLQELGKYEQYIKWP | |
| 6 | VSSQCVNLTTRTQLPPAYTNSFTRGVYYPDKVFRSSVLHSTQD | SARS-CoV- |
| | LFLPFFSNVTWFHAIHVSGTNGTKRFDNPVLPFNDGVYFASTE | 2 Spike |
| | KSNIIRGWIFGTTLDSKTQSLLIVNNATNVVIKVCEFQFCNDPFL | protein, |
| | GVYYHKNNKSWMESEFRVYSSANNCTFEYVSQPFLMDLEGK | extracellular |
| | QGNFKNLREFVFKNIDGYFKIYSKHTPINLVRDLPQGFSALEPL | domain with |
| | VDLPIGINITRFQTLLALHRSYLTPGDSSSGWTAGAAAYYVGY | mutated furin |
| | LQPRTFLLKYNENGTITDAVDCALDPLSETKCTLKSFTVEKGIY | protease site |
| | QTSNFRVQPTESIVRFPNITNLCPFGEVFNATRFASVYAWNRKR | |
| | ISNCVADYSVLYNSASFSTFKCYGVSPTKLNDLCFTNVYADSF | |
| | VIRGDEVRQIAPGQTGKIADYNYKLPDDFTGCVIAWNSNNLDS | |
| | KVGGNYNYLYRLFRKSNLKPFERDISTEIYQAGSTPCNGVEGF | |
| | NCYFPLQSYGFQPTNGVGYQPYRVVVLSFELLHAPATVCGPK | |
| | KSTNLVKNKCVNFNFNGLTGTGVLTESNKKFLPFQQFGRDIAD | |
| | TTDAVRDPQTLEILDITPCSFGGVSVITPGTNTSNQVAVLYQDV | |
| | NCTEVPVAIHADQLTPTWRVYSTGSNVFQTRAGCLIGAEHVN | |
| | NSYECDIPIGAGICASYQTQTNSPAAAASVASQSIIAYTMSLGA | |
| | ENSVAYSNNSIAIPTNFTISVTTEILPVSMTKTSVDCTMYICGDS | |
| | TECSNLLLQYGSFCTQLNRALTGIAVEQDKNTQEVFAQVKQIY | |
| | KTPPIKDFGGFNFSQILPDPSKPSKRSFIEDLLFNKVTLADAGFIK | |
| | QYGDCLGDIAARDLICAQKFNGLTVLPPLLTDEMIAQYTSALL | |
| | AGTITSGWTFGAGAALQIPFAMQMAYRFNGIGVTQNVLYENQ | |
| | KLIANQFNSAIGKIQDSLSSTASALGKLQDVVNQNAQALNTLV | |
| | KQLSSNFGAISSVLNDILSRLDKVEAEVQIDRLITGRLQSLQTY | |
| | VTQQLIRAAEIRASANLAATKMSECVLGQSKRVDFCGKGYHL | |
| | MSFPQSAPHGVVFLHVTYVPAQEKNFTTAPAICHDGKAHFPRE | |
| | GVFVSNGTHWFVTQRNFYEPQIITTDNTFVSGNCDVVIGIVNN | |
| | TVYDPLQPELDSFKEELDKYFKNHTSPDVDLGDISGINASVVNI | |
| | QKEIDRLNEVAKNLNESLIDLQELGKYEQYIKWP | |
| 7 | MSDNGPQNQRNAPRITFGGPSDSTGSNQNGERSGARSKQRRPQ | SARS-CoV- |
| | GLPNNTASWFTALTQHGKEDLKFPRGQGVPINTNSSPDDQIGY | 2 |
| | YRRATRRIRGGDGKMKDLSPRWYFYYLGTGPEAGLPYGANK | Nucleocapsid |
| | DGIIWVATEGALNTPKDHIGTRNPANNAAIVLQLPQGTTLPKG | |
| | FYAEGSRGGSQASSRSSSRSRNSSRNSTPGSSRGTSPARMAGN | |
| | GGDAALALLLLDRLNQLESKMSGKGQQQQGQTVTKKSAAEA | |
| | SKKPRQKRTATKAYNVTQAFGRRGPEQTQGNFGDQELIRQGT | |
| | DYKHWPQIAQFAPSASAFFGMSRIGMEVTPSGTWLTYTGAIKL | |
| | DDKDPNFKDQVILLNKHIDAYKTFPPTEPKKDKKKKADETQA | |
| | LPQRQKKQQTVTLLPAADLDDFSKQLQQSMSSADSTQA | |
| 8 | MSDNGPQNQRNAPRITFGGPSDSTGSNQNGERSGARSKQRRPQ | SARS-CoV- |
| | GLPNNTASWFTALTQHGKEDLKFPRGQGVPINTNSSPDDQIGY | 2 |
| | YRRATRRIRGGDGKMKDLSPRWYFYYLGTGPEAGLPYGANK | Nucleocapsid |
| | DGIIWVATEGALNTPKDHIGTRNPANNAAIVLQLPQGTTLPKG | with deleted |
| | FYAEGSRGGSQASSRSSSRSRNSSRNSTPGSSRGTSPARMAGN | dimerization |
| | GGDAALALLLLDRLNQLESKMSGKGQQQQGQTVTKKSAAEA | domain with |
| | SKKGSGSTFPPTEPKKDKKKKADETQALPQRQKKQQTVTLLP | glycine- |
| | AADLDDFSKQLQQSMSSADSTQA | serine linker |
| | | in place |
| 9 | MYSFVSEETGTLIVNSVLLFLAFVVFLLVTLAILTALRLCAYCC | SARS-CoV- |
| | NIVNVSLVKPSFYVYSRVKNLNSSRVPDLLV | 2 Envelope |
| | | protein |
| 10 | MADSNGTITVEELKKLLEQWNLVIGFLFLTWICLLQFAYANRN | SARS-CoV- |
| | RFLYIIKLIFLWLLWPVTLACFVLAAVYRINWITGGIAIAMACL | 2 Membrane |
| | VGLMWLSYFIASFRLFARTRSMWSFNPETNILLNVPLHGTILTR | protein |
| | PLLESELVIGAVILRGHLRIAGHHLGRCDIKDLPKEITVATSRTL | |
| | SYYKLGASQRVAGDSGFAAYSRYRIGNYKLNTDHSSSSDNIAL | |
| | LVQ | |
| 11 | VSSQCVNLTTRTQLPPAYTNSFTRGVYYPDKVFRSSVLHSTQD | SARS-CoV- |
| | LFLPFFSNVTWFHAIHVSGTNGTKRFDNPVLPFNDGVYFASTE | 2 S1 domain |
| | KSNIIRGWIFGTTLDSKTQSLLIVNNATNVVIKVCEFQFCNDPFL | of Spike |
| | GVYYHKNNKSWMESEFRVYSSANNCTFEYVSQPFLMDLEGK | Protein, |
| | QGNFKNLREFVFKNIDGYFKIYSKHTPINLVRDLPQGFSALEPL | extracellular |
| | VDLPIGINITRFQTLLALHRSYLTPGDSSSGWTAGAAAYYVGY | |
| | LQPRTFLLKYNENGTITDAVDCALDPLSETKCTLKSFTVEKGIY | |
| | QTSNFRVQPTESIVRFPNITNLCPFGEVFNATRFASVYAWNRKR | |
| | ISNCVADYSVLYNSASFSTFKCYGVSPTKLNDLCFTNVYADSF | |
| | VIRGDEVRQIAPGQTGKIADYNYKLPDDFTGCVIAWNSNNLDS | |
| | KVGGNYNYLYRLFRKSNLKPFERDISTEIYQAGSTPCNGVEGF | |
| | NCYFPLQSYGFQPTNGVGYQPYRVVVLSFELLHAPATVCGPK | |
| | KSTNLVKNKCVNFNFNGLTGTGVLTESNKKFLPFQQFGRDIAD | |
| | TTDAVRDPQTLEILDITPCSFGGVSVITPGTNTSNQVAVLYQDV | |
| | NCTEVPVAIHADQLTPTWRVYSTGSNVFQTRAGCLIGAEHVN | |
| | NSYECDIPIGAGICASYQTQTNSP | |
| 12 | SVASQSIIAYTMSLGAENSVAYSNNSIAIPTNFTISVTTEILPVS | SARS-CoV- |
| | MTKTSVDCTMYICGDSTECSNLLLQYGSFCTQLNRALTGIAVEQ | 2 S2 domain |
| | DKNTQEVFAQVKQIYKTPPIKDFGGFNFSQILPDPSKPSKRSFIE | of Spike |
| | DLLFNKVTLADAGFIKQYGDCLGDIAARDLICAQKFNGLTVLP | Protein, |
| | PLLTDEMIAQYTSALLAGTITSGWTFGAGAALQIPFAMQMAYR | extracellular |
| | FNGIGVTQNVLYENQKLIANQFNSAIGKIQDSLSSTASALGKLQ | |
| | DVVNQNAQALNTLVKQLSSNFGAISSVLNDILSRLDKVEAEVQ | |
| | IDRLITGRLQSLQTYVTQQLIRAAEIRASANLAATKMSECVLGQ | |
| | SKRVDFCGKGYHLMSFPQSAPHGVVFLHVTYVPAQEKNFTTA | |
| | PAICHDGKAHFPREGVFVSNGTHWFVTQRNFYEPQIITTDNTF | |
| | VSGNCDVVIGIVNNTVYDPLQPELDSFKEELDKYFKNHTSPDV | |
| | DLGDISGINASVVNIQKEIDRLNEVAKNLNESLIDLQELGKYEQ | |
| | YIKWP | |
| 13 | SVASQSIIAYTMSLGAENSVAYSNNSIAIPTNFTISVTTEILPVSM | SARS-CoV- |
| | TKTSVDCTMYICGDSTECSNLLLQYGSFCTQLNRALTGIAVEQ | 2 S2 domain |
| | DKNTQEVFAQVKQIYKTPPIKDFGGFNFSQILPDPSKPSKRSFIE | of Spike |
| | DLLFNKVTLADAGFIKQYGDCLGDIAARDLICAQKFNGLTVLP | Protein, |
| | PLLTDEMIAQYTSALLAGTITSGWTFGAGAALQIPFAMQMAYR | extracellular, |
| | FNGIGVTQNVLYENQKLIANQFNSAIGKIQDSLSSTASALGKLQ | stabilized |
| | DVVNQNAQALNTLVKQLSSNFGAISSVLNDILSRLDPPEAEVQI | |
| | DRLITGRLQSLQTYVTQQLIRAAEIRASANLAATKMSECVLGQ | |
| | SKRVDFCGKGYHLMSFPQSAPHGVVFLHVTYVPAQEKNFTTA | |
| | PAICHDGKAHFPREGVFVSNGTHWFVTQRNFYEPQIITTDNTF | |
| | VSGNCDVVIGIVNNTVYDPLQPELDSFKEELDKYFKNHTSPDV | |
| | DLGDISGINASVVNIQKEIDRLNEVAKNLNESLIDLQELGKYEQ | |
| | YIKWP | |
| 14 | PNITNLCPFGEVFNATRFASVYAWNRKRISNCVADYSVLYNSA | SARS-CoV- |
| | SFSTFKCYGVSPTKLNDLCFTNVYADSFVIRGDEVRQIAPGQTG | 2 Receptor |
| | KIADYNYKLPDDFTGCVIAWNSNNLDSKVGGNYNYLYRLFRK | Binding |
| | SNLKPFERDISTEIYQAGSTPCNGVEGFNCYFPLQSYGFQPTNG | Domain |
| | VGYQPYRVVVLSFELLHAPATV | (RBD), 330- |
| | | 524 |
| 15 | RVQPTESIVRFPNITNLCPFGEVFNATRFASVYAWNRKRISNCV | SARS-CoV- |
| | ADYSVLYNSASFSTFKCYGVSPTKLNDLCFTNVYADSFVIRGD | 2 Receptor |
| | EVRQIAPGQTGKIADYNYKLPDDFTGCVIAWNSNNLDSKVGG | Binding |
| | NYNYLYRLFRKSNLKPFERDISTEIYQAGSTPCNGVEGFNCYFP | Domain |
| | LQSYGFQPTNGVGYQPYRVVVLSFELLHAPATVCGPKKSTNL | (RBD), 319- |
| | VKNKCVNFNFNGLTGT | 541 |
| 16 | PNITNLCPFGEVFNATRFASVYAWNRKRISNCVADYSVLYNSA | SARS-CoV- |
| | SFSTFKCYGVSPTKLNDLCFTNVYADSFVIRGDEVRQIAPGQTG | 2 RBD-2E8 |
| | KIADYNYKLPDDFTGCVIAWNSNNLDSKVGGNYNYLYRLFRK | scFv fusion |
| | SNLKPFERDISTEIYQAGSTPCNGVEGFNCYFPLQSYGFQPTNG | protein |
| | VGYQPYRVVVLSFELLHAPATVTNLVKNKSVNSNSNGLTGTG | |
| | SGSGQVQLKESGPGLVAPSQSLSITCTVSGFSLSGYSVHWVRQ | |
| | PPGKGLEWLGMIWGGGNTDYKSALKSRLTISKDNSRSQVLLK | |
| | MNSLQIDDTAIYYCARNYGYSPFVHWGQGTLVTVSAGGGGSG | |
| | GGGSGGGGSDIVMTQSPSSLAMSVGQKVTMSCKSSQSLLNSD | |
| | NQKNYLAWYQQKPGQSPKLLVYFASSRESGVSDRFIGSGSGT | |
| | DFTLTIGSVQSEDLAYYFCQQLYRTPFTFGSGTKLEIK | |
| 17 | QVKLVQSGPELVKPGASVKMSCKASGYTFTNYVMHWVKQQP | B6 scFv- |
| | GQVLEWIGYINPYNDDTKYNEKFKGKATLASDKSSNTAYMEL | human CH1- |
| | SSLPSEDSAVYYCASGGYYTMDYWGQGTSVTVSSGGGGSGG | RBD |
| | GGSGGGGSDIVLTQSPLSLPVSLGDQASISCRSSQSLVHSKGYT | fusion |
| | YLHWYLQKPGQSPKLLIYKVSRRFSGVPDRFNGSGSGTDFTLK | protein |
| | ISRVEAEDLGVYFCSQSTHVPYTFGGGTKLELKRAASTKGPSV | |
| | FPLAPSSKSTSGGTAALGCLVKDYFPEPVTVSWNSGALTSGV | |
| | HTFPAVLQSSGLYSLSSVVTVPSSSLGTQTYICNVNHKPSNTKV | |
| | DKKVEPKSGSGGGSGGSGGRVQPTESIVRFPNITNLCPFGEVFN | |
| | ATRFASVYAWNRKRISNCVADYSVLYNSASFSTFKCYGVSPTK | |
| | LNDLCFTNVYADSFVIRGDEVRQIAPGQTGKIADYNYKLPDDF | |
| | TGCVIAWNSNNLDSKVGGNYNYLYRLFRKSNLKPFERDISTEI | |
| | YQAGSTPCNGVEGFNCYFPLQSYGFQPTNGVGYQPYRVVVLS | |
| | FELLHAPATVCGPKKSTNLVKNKCVNFNFNGLTGT | |
| 18 | QVKLQQSGAELVKPGASVKLSCKASGYTFNSYFMHWMKQRP | 10F7 scFv- |
| | VQGLEWIGMIRPNGGTTDYNEKFKNKATLTVDKSSNTAYMQL | human CH1 |
| | NSLTSGDSAVYYCARWEGSYYALDYWGQGTTVTVSSGGGGS | Nucleocapsid |
| | GGGGSSGGGGSSDIELTQSPAIMSATLGEKVTMTCRASSNVKY | (mutant) |
| | MYWYQQKSGASPKLWIYYTSNLASGVPGRFSGSGSGTSYSLTI | fusion |
| | SSVEAEDAATYYCQQFTSSPYTFGGGTKLEIKRAAAASTKGPS | protein |
| | VFPLAPSSKSTSGGTAALGCLVKDYFPEPVTVSWNSGALTSGV | |
| | HTFPAVLQSSGLYSLSSVVTVPSSSLGTQTYICNVNHKPSNTKV | |
| | DKKVEPKSGSGGGSGGSGGMSDNGPQNQRNAPRITFGGPSDS | |
| | TGSNQNGERSGARSKQRRPQGLPNNTASWFTALTQHGKEDLK | |
| | FPRGQGVPINTNSSPDDQIGYYRRATRRIRGGDGKMKDLSPRW | |
| | YFYYLGTGPEAGLPYGANKDGIIWVATEGALNTPKDHIGTRNP | |
| | ANNAAIVLQLPQGTTLPKGFYAEGSRGGSQASSRSSSRSRNSSR | |
| | NSTPGSSRGTSPARMAGNGGDAALALLLLDRLNQLESKMSGK | |
| | GQQQQGQTVTKKSAAEASKKGSGSTFPPTEPKKDKKKKADET | |
| | QALPQRQKKQQTVTLLPAADLDDFSKQLQQSMSSADSTQA | |
| 19 | QVQLKESGPGLVAPSSQLSITCTVSGFSLSGYSVHWVRQPPGK | 2E8 scFv- |
| | GLEWLGMIWGGGNTDYKSALKSRLTISKDNSRSQVLLKMNSL | mouse CH1- |
| | QIDDTAIYYCARNYGYSPFVHWGQGTLVTVSAGGGGSGGGGS | S1 Spike |
| | GGGGSDIVMTQSPSSLAMSVGQKVTMSCKSSQSLLNSDNQKN | |
| | YLAWYQQKPGQSPKLLVYFASSRESGVSDRFIGSGSGTDFTLTI | |
| | GSVQSEDLAYYFCQQLYRTPFTFGSGTKLEIKAKTTPPSVYPLA | |
| | PGSAAQTNSMVTLGCLVKGYFPEPVTVTWNSGSLSSGVHTFP | |
| | AVLQSDLYTLSSSVTVPSSTWPSETVTCNVAHPASSTKVDKKI | |
| | VPRDGTSGGSGGSGGVSSQCVNLTTRTQLPPAYTNSFTRGVYY | |
| | PDKVFRSSVLHSTQDLFLPFFSNVTWFHAIHVSGTNGTKRFDN | |
| | PVLPFNDGVYFASTEKSNIIRGWIFGTTLDSKTQSLLIVNNATN | |
| | VVIKVCEFQFCNDPFLGVYYHKNNKSWMESEFRVYSSANNCT | |
| | FEYVSQPFLMDLEGKQGNFKNLREFVFKNIDGYFKIYSKHTPIN | |
| | LVRDLPQGFSALEPLVDLPIGINITRFQTLLALHRSYLTPGDSSS | |
| | GWTAGAAAYYVGYLQPRTFLLKYNENGTITDAVDCALDPLSE | |
| | TKCTLKSFTVEKGIYQTSNFRVQPTESIVRFPNITNLCPFGEVFN | |
| | ATRFASVYAWNRKRISNCVADYSVLYNSASFSTFKCYGVSPTK | |
| | LNDLCFTNVYADSFVIRGDEVRQIAPGQTGKIADYNYKLPDDF | |
| | TGCVIAWNSNNLDSKVGGNYNYLYRLFRKSNLKPFERDISTEI | |
| | YQAGSTPCNGVEGFNCYFPLQSYGFQPTNGVGYQPYRVVVLS | |
| | FELLHAPATVCGPKKSTNLVKNKCVNFNFNGLTGTGVLTESN | |
| | KKFLPFQQFGRDIADTTDAVRDPQTLEILDITPCSFGGVSVITPG | |
| | TNTSNQVAVLYQDVNCTEVPVAIHADQLTPTWRVYSTGSNVF | |
| | QTRAGCLIGAEHVNNSYECDIPIGAGICASYQTQTNSP | |
| 20 | QVQLQESGGGSVQAGGSLRLSCVASGYTDSTYCVGWFRQAPG | IH4-mouse |
| | KEREGVARINTISGRPWYADSVKGRFTISQDNSKNTVFLQMNS | CH1-S2 |
| | LKPEDTAIYYCTLTTANSRGFCSGGYNYKGQGQVTVSAKTTP | spike |
| | PSVYPLAPGSAAQTNSMVTLGCLVKGYFPEPVTVTWNSGSLSS | |
| | GVHTFPAVLQSDLYTLSSSVTVPSSTWPSETVTCNVAHPASSK | |
| | VDKKIVPRDGTSGGSGGSGGSVASQSIIAYTMSLGAENSVAYS | |
| | NNSIAIPTNFTISVTTEILPVSMTKTSVDCTMYICGDSTECSNLL | |
| | LQYGSFCTQLNRALTGIAVEQDKNTQEVFAQVKQIYKTPPIKD | |
| | FGGFNFSQILPDPSKPSKRSFIEDLLFNKVTLADAGFIKQYGDCL | |
| | GDIAARDLICAQKFNGLTVLPPLLTDEMIAQYTSALLAGTITSG | |
| | WTFGAGAALQIPFAMQMAYRFNGIGVTQNVLYENQKLIANQF | |
| | NSAIGKIQDSLSSTASALGKLQDVVNQNAQALNTLVKQLSSNF | |
| | GAISSVLNDILSRLDKVEAEVQIDRLITGRLQSLQTYVTQQLIRA | |
| | AEIRASANLAATKMSECVLGQSKRVDFCGKGYHLMSFPQSAP | |
| | HGVVFLHVTYVPAQEKNFTTAPAICHDGKAHFPREGVFVSNG | |
| | THWFVTQRNFYEPQIITTDNTFVSGNCDVVIGIVNNTVYDPLQP | |
| | ELDSFKEELDKYFKNHTSPDVDLGDISGINASVVNIQKEIDRLN | |
| | EVAKNLNESLIDLQELGKYEQYIKWP | |
| 35 | MGWSCIILFLVATATGVHS | 10F7-RBD; |
| | QVKLQQSGAELVKPGASVKLSCKASGYTFNSYFMHWMKQ | underline |
| | RPVQGLEWIGMIRPNGGTTDYNEKFKNKATLTVDKSSNTA | indicates the |
| | YMQLNSLTSGDSAVYYCARWEGSYYALDYWGQGTTVTVS | leader |
| | SGGGGSGGGGSSGGGGSSDIELTQSPAIMSATLGEKVTMTCR | peptide |
| | ASSNVKYMYWYQQKSGASPKLWIYYTSNLASGVPGRFSGS | sequence; |
| | GSGTSYSLTISSVEAEDAATYYCQQFTSSPYTFGGGTKLEIK | underline and |
| | RAAAsstgsggsgNITNLCPFGEVFNATRFASVYAWNRKRISNC | italics |
| | VADYSVLYNSASFSTFKCYGVSPTKLNDLCFTNVYADSFVIR | indicates |
| | GDEVRQIAPGQTGKIADYNYKLPDDFTGCVIAWNSNNLDS | linker |
| | KVGGNYNYLYRLFRKSNLKPFERDISTEIYQAGSTPCNGVE | sequences; |
| | GFNCYFPLQSYGFQPTNGVGYQPYRVVVLSFELLHAPATV | bold |
| | CGPKKHHHHHH | indicates the |
| | | scFv |
| | | sequences; |
| | | bold |
| | | underline |
| | | indicates the |
| | | RBD |
| | | sequence; |
| | | italics |
| | | indicates the |
| | | His-tag; |
| | | lowercase |
| | | sequence |
| | | indicates a |
| | | linker |
| | | sequence |
| 36 | NITNLCPFGEVFNATRFASVYAWNRKRISNCVADYSVLYNSAS | South Africa |
| | FSTFKCYGVSPTKLNDLCFTNVYADSFVIRGDEVRQIAPGQTG | Variant- |
| | NIADYNYKLPDDFTGCVIAWNSNNLDSKVGGNYNYLYRLFRK | B.1.351 |
| | SNLKPFERDISTEIYQAGSTPCNGVKGFNCYFPLQSYGFQPTYG | RBD |
| | VGYQPYRVVVLSFELLHAPATVCGPKK | sequence |
| 37 | NITNLCPFGEVFNATRFASVYAWNRKRISNCVADYSVLYNSAS | Brazil |
| | FSTFKCYGVSPTKLNDLCFTNVYADSFVIRGDEVRQIAPGQTG | Variant-P.1 |
| | TIADYNYKLPDDFTGCVIAWNSNNLDSKVGGNYNYLYRLFRK | RBD |
| | SNLKPFERDISTEIYQAGSTPCNGVKGFNCYFPLQSYGFQPTYG | sequence |
| | VGYQPYRVVVLSFELLHAPATVCGPKK | |
| 38 | MDWIWRILFLVGAATGAHSPNITNLCPFGEVFNATRFASVYA | RBD-2E8 |
| | WNRKRISNCVADYSVLYNSASFSTFKCYGVSPTKLNDLCFT | Fusion |
| | NVYADSFVIRGDEVRQIAPGQTGKIADYNYKLPDDFTGVIA | Protein; |
| | WNSNNLDSKVGGNYNYLYRLFRKSNLKPFERDISTEIYQAG | underline |
| | STPCNGVEGFNCYFPLQSYGFQPTNGVGYQYRVVVLSFELL | indicates the |
| | HAPATVTNLVKNKSVNSNSNGLTGTGSGSGQVQLKESGPGLVA | leader |
| | PSQSLSITCTVSGFSLSGYSVHWVRQPPGKGLEWLGMIWG | peptide |
| | GGNTDYKSALKSRLTISKDNSRSQVLLKMNSLQIDDTAIYY | sequence; |
| | CARNYGYSPFVHWGQGTLVTVSAGGGGWSGGGGSGGGG | bold |
| | SDIVMTQSPSSLAMSVGQKVTMSCKSSQSLLNSDNQKNYLA | underline |
| | WYQQKPGQSPKLLVYFASSRESGVSDRFIGSGSGTDFTLTI | indicates the |
| | GSVQSEDLAYYFCQQLYRTPFTFGSGTKLEIKHHHHHH | RBD |
| | | sequence; |
| | | underline and |
| | | italics |
| | | indicate |
| | | Spike |
| | | protein, |
| | | glycine- |
| | | serine linker; |
| | | bold |
| | | indicates the |
| | | scFv |
| | | sequences; |
| | | italics |
| | | indicates the |
| | | His-tag |
| 39 | MQVKLVQSGPELVKPGASVKMSCKASGYTFTNYVMHWV | B6-CH1- |
| | KQQPGQVLEWIGYINPYNDDTKYNEKFKGKATLASDKSSN | RBD fusion |
| | TAYMELSSLPSEDSAVYYCASGGYYTMDYWGQGTSVTVSS | protein; bold |
| | GGGGSGGGGSGGGGSDIVLTQSPLSLPVSLGDQASISCRSS | indicates the |
| | QSLVHSKGTYYLHWYLQKPGQSPKLLIYKVSRRFSGVPDR | scFv |
| | FNGSGSGTDFTLKISRVEAEDLGVYFCSQSTHVPYTFGGGT | sequences; |
| | KELKRAASTKGPSVFPLAPSSKSTSGGTAALGCLVKDYFPEPV | underline |
| | TVSWNSGALTSGVHTFPAVLQSSGLYSLSSVVTVPSSSLGTQT | indicates |
| | YICNVNHKPSNTKVDKKVEPKSGSGGGSGGSGGRVQPTESIVR | CH1 |
| | FPNITNLCPFGEVFNATRFASVYAWNRKRISNCVADYSVLY | antibody |
| | NSASFSTFKCYGVSPTKLNDLCFTNVYADSFVIRGDEVRQIA | domain |
| | PGQTGKIADYNYKLPDDFTGCVIAWNSNNLDSKVGGNYNY | linker |
| | LYRLFRKSNLKPFERDISTEIYQAGSTPCNGVEGFNCYFPLQ | sequence; |
| | SYGFQPTNGVGYQPYRVVVLSFELLHAPATVCGPKKSTNL | underline and |
| | VKNKCVNFNFNGLTGTGGSGG hhhhhh | italics |
| | | indicate |
| | | linker |
| | | sequence; |
| | | bold |
| | | underline |
| | | indicates the |
| | | RBD |
| | | sequence; |
| | | lowercase |
| | | indicates |
| | | His-tag |
| | | sequence |
| 42 | QVQLQESGGGSVQAGGSLRLSCVASGYTDSTYCVGWFRQ | IH4-RBD |
| | APGKEREGVARINTISGRPWYADSVKGRFTISQDNSKNTVF | fusion |
| | LQMNSLKPEDTAIYYCTLTTANSRGFCSGGYNYKGQGTQV | protein; bold |
| | TVSSASTGSGGSGNITNLCPFGEVFNATRFASVYAWNRKRIS | indicates the |
| | NCVADYSVLYNSASFSTFKCYGVSPTKLNDLCFTNVYADSF | scFv |
| | VIRGDEVRQIAPGQTGKIADYNYKLPDDFTGCVIAWNSNNL | sequence; |
| | DSKVGGNYNYLYRLFRKSNLKPFERDISTEIYQAGSTPCNG | italics |
| | VEGFNCYFPLQSYGFQPTNGVGYQPYRVVVLSFELLHAPA | indicates the |
| | TVCGPKKHHHHHH | linker |
| | | sequence; |
| | | bold |
| | | underline |
| | | indicates the |
| | | RBD |
| | | sequence; |
| | | and underline |
| | | indicates the |
| | | his tag |

In some aspects, the first domain can be one or more of SEQ TD NOs: 5-15, 36 or 37 or an antigenic fragment thereof, the optional linker can be one of SEQ TD NOs: 21-24; and the second domain can one of SEQ TD Nos: 1-4. In some aspects, the first domain can be one or more of SEQ TD Nos: 1-4; the optional linker can be one of SEQ TD NOs: 21-24; and the second domain can be one or more of SEQ TD NOs: 5-15, 36 or 37 or an antigenic fragment thereof. In some aspects, the first domain can SEQ TD NO: 14, and the second domain can be SEQ TD NO: 1. In some aspects, the first domain can be SEQ ID NO: 15, and the second domain can be SEQ TD NO: 2. In some aspects, the first domain can be SEQ ID NO: 15, the second domain can be SEQ TD NO: 2, and the linker can be SEQ TD NO: 23. In some aspects, the first domain can be SEQ TD NO: 7, and the second domain can be SEQ TD NO: 3. In some aspects, the first domain can be SEQ TD NO: 7, the second domain can be SEQ ID NO: 3, and the linker can be SEQ TD NO: 23. In some aspects, the first domain can be SEQ TD NO: 8, and the second domain can be SEQ ID NO: 3. In some aspects, the first domain can be SEQ ID NO: 8, the second domain can be SEQ ID NO: 3, and the linker can be SEQ ID NO: 23. In some aspects, the first domain can be SEQ ID NO: 10, and the second domain can be SEQ ID NO: 1. In some aspects, the first domain can be SEQ ID NO: 10, the second domain can be SEQ ID NO: 1, and the linker can be SEQ ID NO: 22 In some aspects, the first domain can be SEQ ID NO: 10, and the second domain can be SEQ ID NO: 4. In some aspects, the first domain can be SEQ ID NO: 10, the second domain can be SEQ ID NO: 4, and the linker can be SEQ ID NO: 22. In some aspects, the recombinant polypeptide can be any of SEQ ID NOs: 16-20, 35, 38, 39 and 42. In some aspects, the recombinant polypeptide can be any of SEQ ID NOs: 16-20, 35, 38, 39, and 42 without the label or detection tag and secretion sequence.

In some aspects, the recombinant polypeptide can also be flanked by one or more amino acid residues (e.g., glycine residues) at one or both of the N-terminus and C-terminus ends.

In some aspects, the first domain sequence and/or the second domain sequence can exhibit a certain degree of identity or homology to sequence that is derived from. The degree of identity can vary and be determined by methods known to one of ordinary skill in the art. The terms “homology” and “identity” each refer to sequence similarity between two polypeptide sequences. Homology and identity can each be determined by comparing a position in each sequence which can be aligned for purposes of comparison. When a position in the compared sequence is occupied by the same amino acid residue, then the polypeptides can be referred to as identical at that position; when the equivalent site is occupied by the same amino acid (e.g., identical) or a similar amino acid (e.g., similar in steric and/or electronic nature), then the molecules can be referred to as homologous at that position. A percentage of homology or identity between sequences is a function of the number of matching or homologous positions shared by the sequences. The first domain and/or the second domain of a recombinant polypeptide described herein can have at least or about 25%, 50%, 65%, 75%, 80%, 85%, 90%, 95%, 96%, 97%, 98%, or 99% identity or homology to a naturally occurring viral antigen or a red blood cell antigen, respectively.

Disclosed herein are the recombinant polypeptides comprising a domain (referred to herein as the second domain) that binds to the red blood cell surface and another domain (referred to herein as the first domain) that binds to a coronaviral antigen. The first and second domain can be connected by a linker, creating a functionally bispecific fusion protein that can simultaneously bind red blood cells and coronavirus antigen.

Disclosed herein are recombinant polypeptides that comprise two scFv fragments that can be connected via a linker. For example, one of the scFv fragments can target and bind to a red blood cell antigen, such as the H antigen or the glycophorin A antigen, while the other scFv fragment can target and bind to a coronavirus antigen such as the spike protein or the nucleocapsid protein. In some aspects, the coronavirus spike protein and the coronavirus nucleocapsid can be targeted with scFv's or nanobodies.

In some aspects, the first domain can be a moiety that is capable of specifically binding a coronavirus antigen. As such, also disclosed herein are recombinant polypeptides comprising: a first domain, wherein the first domain is a moiety that is capable of specifically binding a coronavirus antigen; a linker; and a second domain, wherein the second domain is a moiety that is capable of specifically binding an antigen on the surface of a red blood cell.

In some aspects, the coronavirus antigen can be a severe acute respiratory syndrome coronavirus 2 (SARS-CoV-2), a severe acute respiratory syndrome (SARS), middle east respiratory syndrome (MERS), a human coronavirus 229E, a human coronavirus NL63, Miniopterus bat coronavirus 1, a Miniopterus bat coronavirus HKU8, porcine epidemic diarrhea virus, a Rhinolophus bat coronavirus HKU2, a Scotophilus bat coronavirus 512, a bovine coronavirus, a human coronavirus OC43, a human coronavirus HKU1, murine coronavirus, a Pipistrellus bat coronavirus HKU5, a Rousettus bat coronavirus HKU9, a Tylonycteris bat coronavirus HKU4, a hedgehog coronavirus 1, an infectious bronchitis virus, a beluga whale coronavirus SW1, an infectious bronchitis virus, a Bulbul coronavirus HKU11, a pangolin coronavirus, a porcine coronavirus HKU15, a WIV1-CoV, a SHC014-CoV, a bat-SL-CoVZC45, a bat-SLCoVZXC21, a SARS-CoVGZ02, a BtKY72, a WIV16, Rs4231, a Rs7327, a Rs9401, a BtRs-BetaCoV/YN2018R, a BtRs-BetaCoV/YN2013, Anlong-112, a Rf2092, a BtRs-BetaCoV/YN2018C, a As6526, Rs4247, a BtRs-BetaCoV/GX2013, a Yunnan2011, a BtRl-BetaCoV/SC2018, a Shannxi2011, a BtRs-BetaCoV/HuB2013, a Bat_CoV_279/2005, a HKU3-12, a HKU3-3, a HKU3-7, a Longquan-140, or a RaTG13 antigen. In some aspects, the coronavirus antigen can be a variant of severe acute respiratory syndrome coronavirus 2 (SARS-CoV-2), a severe acute respiratory syndrome (SARS), middle east respiratory syndrome (MERS), a human coronavirus 229E, a human coronavirus NL63, Miniopterus bat coronavirus 1, a Miniopterus bat coronavirus HKU8, porcine epidemic diarrhea virus, a Rhinolophus bat coronavirus HKU2, a Scotophilus bat coronavirus 512, a bovine coronavirus, a human coronavirus OC43, a human coronavirus HKU1, murine coronavirus, a Pipistrellus bat coronavirus HKU5, a Rousettus bat coronavirus HKU9, a Tylonycteris bat coronavirus HKU4, a hedgehog coronavirus 1, an infectious bronchitis virus, a beluga whale coronavirus SW1, an infectious bronchitis virus, a Bulbul coronavirus HKU11, a pangolin coronavirus, a porcine coronavirus HKU15, a WIV1-CoV, a SHC014-CoV, a bat-SL-CoVZC45, a bat-SLCoVZXC21, a SARS-CoVGZ02, a BtKY72, a WIV16, Rs4231, a Rs7327, a Rs9401, a BtRs-BetaCoV/YN2018R, a BtRs-BetaCoV/YN2013, Anlong-112, a Rf2092, a BtRs-BetaCoV/YN2018C, a As6526, Rs4247, a BtRs-BetaCoV/GX2013, a Yunnan2011, a BtRl-BetaCoV/SC2018, a Shannxi2011, a BtRs-BetaCoV/HuB2013, a Bat_CoV_279/2005, a HKU3-12, a HKU3-3, a HKU3-7, a Longquan-140, a RaTG13 antigen or a variant thereof.

In some aspects, the coronavirus antigen can comprise a sequence from a coronavirus spike (S) protein, envelope (E) protein, a membrane (M) protein, a nucleocapsid (N) protein, an antigenic fragment thereof, or a combination thereof. In some aspects, the coronavirus spike (S) protein can comprise a S1 domain, a S2 domain, the N-terminal domain, a receptor-binding domain or the entire S protein ectodomain. In some aspects, the antigenic fragment thereof can comprise an immunodominant epitope from coronavirus spike (S) protein, envelope (E) protein, a membrane (M) protein, or nucleocapsid (N) protein.

In some aspects, the first domain can be a single chain variable fragment (scFv), a Fab, a camelid antibody, a nanobody, a shark vNAR antibody, adnectins, anticalins, avimers, Fynomers, Kunitz domains, knottins, affibodies, β-hairpin mimetics, and designed ankyrin repeat proteins. In some aspects, the first domain can be a peptide capable of binding to the coronavirus antigen or a portion of an angiotensin converting enzyme-2 (ACE2) protein receptor that binds to the coronavirus spike protein. ACE2 is a protein receptor that, for example, a coronavirus can bind to leading to an infection of one or more cells. For example, using sequences that the coronavirus binds to that result in infection can be exploited such that they are used as a capture ligand that can bind to the coronavirus spike protein in the methods disclosed herein. In some aspects, the portion of the ACE2 protein receptor that binds to the coronavirus spike protein can be or is derived from human ACE2 (amino acid 18-615), human ACE2 (amino acid 18-740) or human ACE2 (amino acid 18-55) or human ACE2 (amino acid 18-88). For example, the portion of the ACE2 protein receptor can bind to the coronavirus spike protein that is on the surface of SARS-CoV-2 to infect one or more cells. In some aspects, the first domain can be SEQ ID NO: 30. In some aspects, the first domain can be SEQ ID NO: 31. In some aspects, the portion of the ACE2 protein receptor that can bind to the coronavirus spike protein that is on the surface of SARS-CoV-2 to infect one or more cells is SEQ ID NO: 30 or SEQ ID NO: 31.

In some aspects, the scFv can be derived from an antibody capable of specifically binding to a spike protein. In some aspects, wherein the scFv can be derived from CR3022, CR3013, m396, 80R, F26G29, 18F3, 7B11, B38, H4, CA1, CB6, S309, 47D11, 311mab-31B5, 311mab-32D4, 311mab-31B9, H014, 5A6, COV2-2196, COV-2130, COV2-2381, 414-1, P2C-1F11, P2B-2F6, or a P2C-1A3 antibody. In some aspects, CR3022, CR3013, m396, 80R, F26G29, 18F3, 7B11, B38, H4, CA1, CB6, S309, 47D11, 311mab-31B5, 311mab-32D4, 311mab-31B9, H014, 5A6, COV2-2196, COV-2130, COV2-2381, 414-1, P2C-1F11, P2B-2F6, or a P2C-1A3 antibody are capable of binding to a spike protein.

In some aspects, the scFv can be derived from an antibody capable of specifically binding to a nucleocapsid protein. In some aspects, the scFv can be derived from S-A5D5, 18F629.1, P140.20B7, P140.19B6, P140.19C7, S-39-2, S-125-2, S-144-3, S-162-2, N-17-3, N-30-12, CR3009, CR3018, N10E4, N1E8, N8E1, N18, MA2.D5, MA2.D7, MA2.E3, or A17 antibody. In some aspects, the S-A5D5, 18F629.1, P140.20B7, P140.19B6, P140.19C7, S-39-2, S-125-2, S-144-3, S-162-2, N-17-3, N-30-12, CR3009, CR3018, N10E4, N1E8, N8E1, N18, MA2.D5, MA2.D7, MA2.E3, or A17 antibody are capable of binding to a nucleocapsid protein. In some aspects, the scFv can be derived from mBG17, mBG21, mBG22, mBG57, and mBG67.

In some aspects, the scFv can be constructed from the variable domains of the heavy (VH) and light (VL) chain, wherein the C-terminus of the VH can be linked to the N-terminus of the VL using a flexible linker. Examples of flexible linkers are described herein.

In some aspects, the nanobody can bind to the spike protein. In some aspects, the nanobody can be SARS VHH-72 or MERS VHH-55.

In some aspects, the first domain can comprise SEQ ID NOs: 28, 29, 30, 31, 32, 33, 34 or 40.

In some aspects, the first domain can be SEQ ID NOs: 28-34; the optional linker can comprise SEQ ID NOs: 21, 22, 23 or 24; and the second domain can comprise SEQ ID NOs: 1, 2, 3, or 4. In some aspects, the first domain can comprise SEQ ID NOs: 1, 2, 3, or 4; the optional linker can comprise SEQ TD NOs: 21, 22, 23 or 24; and the second domain can comprise SEQ TD NOs: 28-34 or 40.

TABLE 2
Examples of polypeptide sequences.

| SEQ ID | | |
|---|---|---|
| NO: | Sequence | Name |
| 28 | QMQLVQSGTEVKKPGESLKISCKGSGYGFITYWIGWVRQMPG | CR3022 scFv |
| | KGLEWMGIIYPGDSETRYSPSFQGQVTISADKSINTAYLQWSS | |
| | LKASDTAIYYCAGGSGISTPMDVWGQGTTVTVGGGGSGGGGSG | |
| | GGGSDIQLTQSPDSLAVSLGERATINCKSSQSVLYSSINKNYLA | |
| | WYQQKPGQPPKLLIYWASTRESGVPDRFSGSGSGTDFTLTISSL | |
| | QAEDVAVYYCQQYYSTPYTFGQGTKVEIK | |
| 29 | EVQLVESGGGLVQPGGSLRLSCAASGFTVSSNYMSWVRQAPG | CB6 scFv |
| | KGLEWVSVIYSGGSTFYADSVKGRFTISRDNSMNTLFLQMNSL | |
| | RAEDTAVYYCARVLPMYGDYLDYWGQGTLVTVSSGGGGSG | |
| | GGGSGGGGSDIVMTQSPSSLSASVGDRVTITCRASQSISRYLN | |
| | WYQQKPGKAPKLLIYAASSLQSGVPSRFSGSGSGTDFTLTISSL | |
| | QPEDFATYYCQQSYSTPPEYTFGQGTKLEIKR | |
| 30 | QSTIEEQAKTFLDKFNHEAEDLFYQSSLASWNYNTNITEENVQ | ACE2 |
| | NMNNAGDKWSAFLKEQSTLAQMYPLQEIQNLTVKLQLQAL | (amino acids |
| | QQNGSSVLSEDKSKRLNTILNTMSTIYSTGKVCNPDNPQECLLL | residues 18- |
| | EPGLNEIMANSLDYNERLWAWESWRSEVGKQLRPLYEEYVVL | 615) |
| | KNEMARANHYEDYGDYWRGDYEVNGVDGYDYSRGQLIEDV | |
| | EHTFEEIKPLYEHLHAYVRAKLMNAYPSYISPIGCLPAHLLGD | |
| | MWGRFWTNLYSLTVPFGQKPNIDVTDAMVDQAWDAQRIFKE | |
| | AEKFFVSVGLPNMTQGFWENSMLTDPGNVQKAVCHPTAWDL | |
| | GKGDFRILMCTKVTMDDFLTAHHEMGHIQYDMAYAAQPFLL | |
| | RNGANEGFHEAVGEIMSLSAATPKHLKSIGLLSPDFQEDNETEI | |
| | NFLLKQALTIVGTLPFTYMLEKWRWMVFKGEIPKDQWMKKW | |
| | WEMKREIVGVVEPVPHDETYCDPASLFHVSNDYSFIRYYTRTL | |
| | YQFQFQEALCQAAKHEGPLHKCDISNSTEAGQKLFNMLRLGK | |
| | SEPWTLALENVVGAKNMNVRPLLNYFEPLFTWLKDQNKNSFV | |
| | GWSTDWSPYAD | |
| 31 | QSTIEEQAKTFLDKFNHEAEDLFYQSSLASWNYNTNITEENVQ | ACE2 |
| | NMNNAGDKWSAFLKEQSTLAQMYPLQEIQNLTVKLQLQALQ | (amino acids |
| | QNGSSVLSEDKSKRLNTILNTMSTIYSTGKVCNPDNPQECLLLE | residues 18- |
| | PGLNEIMANSLDYNERLWAWESWRSEVGKQLRPLYEEYVVK | 740) |
| | NEMARANHYEDYGDYWRGDYEVNGVDGYDYSRGQLIEDVE | |
| | HTFEEIKPLYEHLHAYVRAKLMNAYPSYISPIGCLPAHLLGDM | |
| | WGRFWTNLYSLTVPFGQKPNIDVTDAMVDQAWDAQRIFKEA | |
| | EKFFVSVGLPNMTQGFWENSMLTDPGNVQKAVCHPTAWDLG | |
| | KGDFRILMCTKVTMDDFLTAHHEMGHIQYDMAYAAQPFLLR | |
| | NGANEGFHEAVGEIMSLSAATPKHLKSIGLLSPDFQEDNETEIN | |
| | FLLKQALTIVGTLPFTYMLEKWRWMVFKGEIPKDQWMKKWE | |
| | MKREIVGVVEPVPHDETYCDPASLFHVSNDYSFIRYYTRTLYQ | |
| | FQFQEALCQAAKHEGPLHKCDISNSTEAGQKLFNMLRLGKSEP | |
| | WTLALENVVGAKNMNVRPLLNYFEPLFTWLKDQNKNSFVGW | |
| | STDWSPYADQSIKVRISLKSALGDKAYEWNDNEMYLFRSSVA | |
| | YAMRQYFLKVKNQMILFGEEDVRVANLKPRISFNFFVTAPKVS | |
| | DIIPRTEVEKAIRMSRSRINDAFRLNDNSLEFLGIQPTLGPPN | |
| | QPPVS | |
| 32 | EVQLVESGGGVVQPGRSLRLSCAASGFTFSDYPMNWVRQAPG | CR3009 scFv |
| | KGLEWVSSISGSGGSTYYADSVKGRFTISRDNSKNTLYLQMNL | |
| | RAEDTAVYYCAKGLFMVTTYAFDYWGQGTLVTVGGGGSGG | |
| | GGSGGGGSELTQSPSSLSASVGDRVTITCRASQSISSYLNWYQQ | |
| | KPGKAPKLLIYAASSLQSGVPSRFSGSGSGTDFTLTISSLQPEDF | |
| | ATYYCQQSYSTPPTFGQGTKVEIK | |
| 33 | EVQLVESGGGLVQPGGSLRLSCAASGFTFSSYAMSWVRQAPG | CR3018 scFv |
| | KGLEWVSAISGSGGSTYYADSVKGRFTISRDNSKNTLYLQMNS | |
| | LRAEDTAVYYCAKFNPFTSFDYWGQGTLVTVGGGGSGGGGS | |
| | GGGGSELTQSPSSLSASVGDRVTITCRASQSISSYLNWYQQKPG | |
| | KAPKLLIYAASSLQSGVPSRFSGSGSGTDFTLTISSLQPEDFATY | |
| | YCQQSYSTPPTFGQGTKVEIK | |
| 34 | SYVLTQPPSVSVAPGKTARIPCGGNNIGSKSVHWYQQKPGQAP | N18 scFv |
| | VLVIYYDSDRPSGIPERFSGSNSGNTATLTISRVEAGDEADYYC | |
| | QVWDRSSDLVVFGGGTKLTVLSGGSTITSYNVYYTKLSSSGTQ | |
| | VQLVQSGAEVKKPGSSVKVSCKASGGTFSSYAISWVRQAPGQ | |
| | GLEWMGGIIPIFGTANYAQKFQGRVTITADESTSTAYMELSSLR | |
| | SEDTAVYYCARGYWGSGYHYYGMDVWGQGTTVTVSSAS | |
| 40 | EVQLVESGGGLVQPGGSLKLSCAASGFTFSNYGMSWVRQTPD | mBG67 |
| | KRLELVATINRNGGSTYYLDSVKVRFTISRDNAKSTLFLQLSSL | scFv, binding |
| | KSDDTAMYYCARIYDFDEDYFDVWGAGTTVTVSSGGGGSGG | to |
| | GGSGGGGSQIVLTQSPAIMSASLGERVTMTCTASSSVSSSYLH | nucleocapsid |
| | WYQQKPGSSPKLWIYSTSNLASGVPARFSGSGSGTSYSLTISSM | protein |
| | EAEDAATYYCLQYHRSPWTFGGGTKLEIK | |

Nucleic acids. Also disclosed herein, are nucleic acids, including DNA and RNA molecules, capable of encoding the recombinant polypeptides disclosed herein. The nucleic acids encoding the recombinant polypeptides disclosed herein can be have at least or about 25%, 5000, 6500, 7500, 80%, 85%, 90%, 95%, 96%, 97%, 98%, 25 or 990% identity to a corresponding naturally occurring nucleic acid sequence.

Vectors. Further disclosed herein, are vectors comprising the disclosed nucleic acids capable of encoding the recombinant polypeptides disclosed herein. In some aspects, the vector can be an expression vectors, especially those for expression in eukaryotic cells. Such vectors can, for example, be viral, plasmid, cosmid, or artificial chromosome (e.g., yeast artificial chromosome) vectors.

The vectors of the invention can advantageously include the recombinant polypeptides described herein. Other elements included in the design of a particular expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein. The vectors described herein can be introduced into cells or tissues by any one of a variety of known methods within the art. The methods include, for example, stable or transient transfection, lipofection, electroporation and infection with recombinant viral vectors. The term “transfecting” or “transfection” is intended to encompass all conventional techniques for introducing nucleic acid into host cells, including calcium phosphate co-precipitation, DEAE-dextran-mediated transfection, lipofection, electroporation and microinjection.

Cells. Another aspect of the invention pertains to host cells into which a nucleic acid construct has been introduced, i.e., a “recombinant host cell.” It is understood that the term “recombinant host cell” refers not only to the particular subject cell but to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell, although eukaryotic cells are preferred. Exemplary eukaryotic cells include mammalian cells (such as Chinese hamster ovary cells (CHO) or COS cells). Other suitable host cells are known to those skilled in the art.

Coronavirus. In some aspects, the first domain can be derived from a viral antigen. In some aspects, the viral antigen can be derived from a coronavirus. In some aspects, the first domain can be derived from a coronavirus. In some aspects, the first domain can be derived from two or more coronaviruses. In some aspects, the two or more coronaviruses can be the same coronavirus or different coronaviruses or a combination thereof. Examples of coronaviruses include but are not limited to severe acute respiratory syndrome coronavirus 2 (SARS-CoV-2), severe acute respiratory syndrome (SARS), middle east respiratory syndrome (MERS), human coronavirus 229E, human coronavirus NL63, Miniopterus bat coronavirus 1, Miniopterus bat coronavirus HKU8, porcine epidemic diarrhea virus, Rhinolophus bat coronavirus HKU2, Scotophilus bat coronavirus 512, bovine coronavirus, human coronavirus OC43, human coronavirus HKU1, murine coronavirus, Pipistrellus bat coronavirus HKU5, Rousettus bat coronavirus HKU9, Tylonycteris bat coronavirus HKU4, hedgehog coronavirus 1, infectious bronchitis virus, beluga whale coronavirus SW1, infectious bronchitis virus, Bulbul coronavirus HKU11, pangolin coronavirus, and porcine coronavirus HKU15. Examples of SARS-like coronaviruses (SL-CoVs) in bats include but are not limited to WIV1-CoV, SHC014-CoV, bat-SL-CoVZC45, bat-SLCoVZXC21, SARS-CoVGZ02, BtKY72, WIV16, Rs4231, Rs7327, Rs9401, BtRs-BetaCoV/YN2018R, BtRs-BetaCoV/YN2013, Anlong-112, Rf2092, BtRs-BetaCoV/YN2018C, As6526, Rs4247, BtRs-BetaCoV/GX2013, Yunnan2011, BtRl-BetaCoV/SC2018, Shannxi2011, BtRs-BetaCoV/HuB2013, Bat_CoV_279/2005, HKU3-12, HKU3-3, HKU3-7, Longquan-140, and RaTG13.

In some aspects, the coronavirus antigen can be derived from a variant of any of the viruses listed herein. In some aspects, the coronavirus antigen can be derived from a coronavirus variant. In some aspects, the coronavirus antigen can be a receptor binding domain derived from a coronavirus variant. In some aspects, the coronavirus variant differs from a canonical virus sequence by one or more amino acid mutations. In some aspects, the receptor binding domain sequence can be derived from a SARS-CoV-2 variant. In some aspects, the SARS-CoV-2 variant can be B1.1.17, P1 and B.1.351. In some aspects, the receptor binding domain sequence derived from a SARS-CoV-2 variant can have at least or about 25%, 50%, 65%, 75%, 80%, 85%, 90%, 95%, 96%, 97%, 98%, or 99% identity or homology to a native or canonical sequence for the same coronaviral protein.

In some aspects, the recombinant polypeptide described herein can further comprise one or more residues positioned at the N-terminus, C-terminus, or both the N-terminus and C-terminus of the recombinant polypeptide. The one or more residues can be glycine, alanine or serine or a combination thereof. The one or more residues described herein can be any residue that reduces immunogenicity.

Linkers. For a given linker within the compositions disclosed herein, sites available for linking can be found on the polypeptides described herein. One of ordinary skill in the art is capable of selecting the appropriate linker.

In some aspects, the linker can be a flexible linker. In some aspects, the flexible linker can be a glycine-serine linker. In some aspects, the glycine-serine linker can have the formula (GGGGS)x (SEQ ID NO: 25). In some aspects, the x can be 1 to 20. In some aspects, the glycine-serine linker can be at least 5 amino acids in length. In some aspects, the glycine-serine linker can be about 5 to about 100 amino acids in length. In some aspects, the flexible linker can be a glycine linker. In some aspects, the glycine linker can be at least 5 amino acids in length. In some aspects, the flexible linker can be an immunoglobulin G hinge region.

In some aspects, the linker can be a rigid linker. Examples of rigid linkers include but are not limited to an alpha helical linker, an immunoglobulin domain, or a fibronectin-type domain. In some aspects, the alpha helical linker can have the formula (EAAAK)x (SEQ ID NO: 26). In some aspects, the x can be 1 to 20. In some aspects, the alpha helical linker can be at least 5 amino acids in length. In some aspects, the alpha helical linker can be about 5 to about 100 amino acids in length. In some aspects, the rigid linker can be one or more of the entire protein domain (e.g., immunoglobulin domain, or a fibronectin-type domain). In some aspects, the immunoglobulin domain can be a constant domain of immunoglobulin A, M, D, E, or G. In some aspects, the immunoglobulin constant domain can include CH1, CH2, CH3, or CL. In some aspects, the immunoglobulin domain can comprise one or more mutations. In some aspects, the mutation can be a mutation that abrogates dimerization and ensures monomer formation of the recombinant polypeptide.

In some aspects, the linker can be SEQ ID NOs: 21, 22, 23, 24, 25 or 26. In some aspects, the linker comprises the sequence of SEQ ID NOs: 21, 22, 23, 24, 25 or 26. Table 3 lists linker sequences.

The linker can be a covalent bond. To form covalent bonds, a chemically reactive group can be used, for instance, that has a wide variety of active carboxyl groups (e.g., esters) where the hydroxyl moiety is physiologically acceptable at the levels required to modify the recombinant polypeptide.

Any of the polypeptides described herein and incorporated into the recombinant polypeptides can be modified to chemically interact with, or to include, a linker as described herein. In some aspects, a linker sequence (or a spacer sequence) can be incorporated between the first domain and the label or detection tag. In some aspects, a linker sequence (or a spacer sequence) can be incorporated between the second domain and the label or detection tag. In some aspects, a linker sequence (or a spacer sequence) can be incorporated between the one or more sequences of the first domain (e.g., two or more epitopes of coronaviruses; or two or more viral antigen sequences, e.g., S protein and N protein) and the label or detection tag.

Chen et al., Adv Drug Deliv Rev. 2013 October; 65(10):1357-69 is hereby incorporated herein by reference for disclosing examples of fusion protein linkers.

In some aspects, the linker can be optional. In any of the embodiments disclosed herein, the linker can be optional. In some aspects, the recombinant polypeptides disclosed herein can be generated without a linker.

TABLE 3
Examples of linker sequences

| SEQ | | |
|---|---|---|
| ID | | |
| NO: | Sequence | Name |
| 21 | GGGGSGGGGSGGGGSGGGGS | Flexible |
| | | glycine-serine |
| | | linker |
| 22 | AKTTPPSVYPLAPGSAAQTN | Mouse CH1 |
| | SMVTLGCLVKGYFPEPVTVT | linker region |
| | WNSGSLSSGVHTFPAVLQSD | with mutated |
| | LYTLSSSVTVPSSTWPSETV | C-terminal |
| | TCNVAHPASSTKVDKKIVPR | cysteine |
| | DGTS | |
| 23 | ASTKGPSVFPLAPSSKSTSG | Human CH1 |
| | GTAALGCLVKDYFPEPVTVS | linker region |
| | WNSGALTSGVHTFPAVLQSS | with mutated |
| | GLYSLSSVVTVPSSSLGTQT | C-terminal |
| | YICNVNHKPSNTKVDKKVEP | cysteine |
| | KSGSG | |
| 24 | TNLVKNKSVNSNSNGLTGTG | Spike protein, |
| | SGSG | glycine-serine |
| | | linker |

Methods of Making Recombinant Polypeptides

As used herein, the term “recombinant polypeptide” refers to a polypeptide comprising a first domain, a linker, and a second domain as described herein. In some aspects, the linker can be optional.

Disclosed herein are techniques that can be used to produce the recombinant polypeptide described herein.

In some aspects, a secretion signal can be included as part of the recombinant polypeptide for secretion into the supernatant of the cells for downstream collection. In some aspects, the secretion signal can be MDWIWRILFLVGAATGAHS (SEQ TD NO: 27) or MGWSCIILFLVATATGVHIS (SEQ TD NO: 41). In some aspects, the secretion signal can be from a human immunoglobulin heavy chain, a human immunoglobulin light chain, a mouse immunoglobulin heavy chain, a mouse immunoglobulin light chain, or a cytokine. Other secretion signals known in the art can also be used.

Design. In some aspects, any of the individual components can be combined to generate a multitude of recombinant polypeptides as described herein.

In some aspects, the recombinant polypeptides described herein can be designed as monomers. In designing the recombinant polypeptides disclosed herein, they should not aggregate in solution or have tendencies to aggregate in solution. The recombinant polypeptides described herein should also not intrinsically agglutinate red blood cells in the absence of anti-coronavirus antibodies. The recombinant polypeptides should not comprise epitopes that naturally react with other antibodies in uninfected individuals. The recombinant polypeptides disclosed herein can be of any amino acid length, as long as they maintain the properties described herein. In some aspects, the recombinant polypeptides disclosed herein can be less than 1000 amino acids in length. Recombinant polypeptides designed to have less than 1000 amino acids in length can ensure stability. The recombinant polypeptides disclosed herein can specifically bind to red blood cell antigens with high affinity to ensure stable cross-linking. In some aspects, the epitopes on viral antigens that are most targeted by human antibodies are displayed in order to maintain small size of the recombinant polypeptide.

In some aspects, the recombinant polypeptide can contain one or more epitopes to bind to an antigen of the coronavirus, and a sequence that is capable of specifically binding to an antigen on the surface of a red blood cell. The recombinant polypeptide sequences selected should not intrinsically induce an autoimmune response (i.e., the sequences should not intrinsically bind to B cell or T cell receptors).

In some aspects, the first domain can comprise a sequence from the coronavirus nucleocapsid protein. In some aspects, the first domain can comprise the nucleocapsid sequence, wherein the dimerization domain is removed to ensure monomeric nucleocapsid protein production.

In some aspects, the moiety that is capable of specifically binding to a coronavirus antigen can be a scFv, wherein the scFv comprises a VL linked to a VH. In some aspects, the VL can be conjugated to the second domain.

In some aspects, the first domain can comprise a sequence of the coronavirus S2 domain of the spike protein. In some aspects, the first domain can comprise the S2 domain of the spike protein, wherein the S2 domain of the spike protein has a mutation of K986P and V987P to stabilize the protein.

In some aspects, the first domain can comprise two or more epitopes or antigens from a coronavirus protein. In some aspects, the first domain can comprise two or more epitopes or antigens from two or more different coronavirus proteins. In some aspects, the first domain can comprise a spike protein sequence and a nucleocapsid protein sequence. When the first domain comprises two or more different epitopes or antigens, the first domain can specifically bind to anticoronavirus antibodies that can bind to two or more proteins.

In some aspects, the polypeptide sequences selected can further be flanked by one or more amino acid residues at the N- and/or C-terminuses. In some aspects, the recombinant polypeptides disclosed herein can further comprise one or more residues positioned at the N-terminus, C-terminus, or both the N-terminus and C-terminus. In some aspects, the one or more residues can be glycine, alanine or serine or a combination thereof.

The recombinant polypeptides and its component parts can be produced by synthetic methods and recombinant techniques used routinely to produce protein from nucleic acids. The recombinant polypeptides can be stored in an unpurified or in an isolated or substantially purified form until later use.

In some aspects, the first domain can comprise a sequence from the receptor binding domain of the coronavirus spike protein. In some aspects, the receptor binding domain can vary slightly in sequence to match the amino acid sequence of a circulating strains of the target coronavirus.

In some aspects, the recombinant polypeptide disclosed herein can be a recombinant fusion protein. It can be expressed in a variety of expression systems (e.g., bacteria (e.g., E. coli), yeast, insect cells, and mammalian cell). Briefly, a plasmid DNA encoding the recombinant fusion protein can be transfected into cells of any of the expression systems described above. After the recombinant polypeptide or recombinant fusion protein is produced in any one of these systems, they can then also be purified, lyophilized and stored until use.

Antibodies. As noted above, the recombinant polypeptide as disclosed herein, can include an antibody, antibody fragment, or a biologically active variant thereof. As is well known in the art, monoclonal antibodies can be made by recombinant DNA. DNA encoding monoclonal antibodies can be readily isolated and sequenced using conventional procedures (e.g., by using oligonucleotide probes that are capable of binding specifically to genes encoding the heavy and light chains of murine antibodies). Libraries of antibodies or active antibody fragments can also be generated and screened using phage display techniques.

In vitro methods are also suitable for preparing monovalent antibodies. As it is well known in the art, some types of antibody fragments can be produced through enzymatic treatment of a full-length antibody. Digestion of antibodies to produce fragments thereof, particularly, Fab fragments, can be accomplished using routine techniques known in the art. For instance, digestion can be performed using papain. Papain digestion of antibodies typically produces two identical antigen binding fragments, called Fab fragments, each with a single antigen binding site, and a residual Fc fragment. Pepsin treatment yields a fragment that has two antigen combining sites and is still capable of cross-linking antigen. Antibodies incorporated into the present bi-functional allosteric protein-drug molecules can be generated by digestion with these enzymes or produced by other methods.

The fragments, whether attached to other sequences or not, can also include insertions, deletions, substitutions, or other selected modifications of particular regions or specific amino acids residues, provided the activity of the antibody or antibody fragment is not significantly altered or impaired compared to the non-modified antibody or antibody fragment. These modifications can provide for some additional property, such as to remove/add amino acids capable of disulfide bonding, to increase its bio-longevity, to alter its secretory characteristics, etc. In any case, the antibody or antibody fragment must possess a bioactive property, such as specific binding to its cognate antigen. Functional or active regions of the antibody or antibody fragment can be identified by mutagenesis of a specific region of the protein, followed by expression and testing of the expressed polypeptide. Such methods are readily apparent to a skilled practitioner in the art and can include site-specific mutagenesis of the nucleic acid encoding the antibody or antibody fragment.

As used herein, the term “antibody” or “antibodies” can also refer to a human antibody and/or a humanized antibody. Many non-human antibodies (e.g., those derived from mice, rats, or rabbits) are naturally antigenic in humans, and thus can give rise to undesirable immune responses when administered to humans. Therefore, the use of human or humanized antibodies in the methods serves to lessen the chance that an antibody administered to a human will evoke an undesirable immune response.

Antibody humanization techniques generally involve the use of recombinant DNA technology to manipulate the DNA sequence encoding one or more polypeptide chains of an antibody molecule. Accordingly, a humanized form of a non-human antibody (or a fragment thereof) is a chimeric antibody or antibody chain (or a fragment thereof, such as an Fv, Fab, Fab′, or other antigen binding portion of an antibody) which contains a portion of an antigen binding site from a non-human (donor) antibody integrated into the framework of a human (recipient) antibody.

The Fv region is a minimal fragment containing a complete antigen-recognition and binding site consisting of one heavy chain and one light chain variable domain. The three CDRs of each variable domain interact to define an antigen-biding site on the surface of the Vh-Vl dimer. Collectively, the six CDRs confer antigen-binding specificity to the antibody. As well known in the art, a “single-chain” antibody or “scFv” fragment is a single chain Fv variant formed when the VH and VL domains of an antibody are included in a single polypeptide chain that recognizes and binds an antigen. Typically, single-chain antibodies include a polypeptide linker between the Vh and Vl domains that enables the scFv to form a desired three-dimensional structure for antigen binding.

To generate a humanized antibody, residues from one or more complementarity determining regions (CDRs) of a recipient (human) antibody molecule are replaced by residues from one or more CDRs of a donor (non-human) antibody molecule that is known to have desired antigen binding characteristics (e.g., a certain level of specificity and affinity for the target antigen). In some instances, Fv framework (FR) residues of the human antibody are replaced by corresponding non-human residues. Humanized antibodies can also contain residues which are found neither in the recipient antibody nor in the imported CDR or framework sequences. Generally, a humanized antibody has one or more amino acid residues introduced into it from a source which is non-human. In practice, humanized antibodies are typically human antibodies in which some CDR residues and possibly some FR residues are substituted by residues from analogous sites in rodent antibodies. Humanized antibodies generally contain at least a portion of an antibody constant region (Fc), typically that of a human antibody.

Methods for humanizing non-human antibodies are well known in the art. For example, humanized antibodies can be generated by substituting rodent CDRs or CDR sequences for the corresponding sequences of a human antibody. Methods that can be used to produce humanized antibodies are also well known in the art.

Configurations. Each part of the recombinant polypeptide, including the first domain, linker, and second domain can be selected independently. One of ordinary skill in the art would understand that the component parts need to be associated in a compatible manner. In some aspects, the linker can be optional. In some aspects, the recombinant polypeptides can be used to detect coronavirus antibodies in a sample from a subject. In some aspects, the recombinant polypeptides can be used to detect one or more coronavirus antigens. In some aspects, the recombinant polypeptides can be used to detect one or more coronavirus virions in a sample.

In some aspects, the first domain can be positioned at the N-terminus and the second domain can be positioned at the C-terminus of the recombinant polypeptide. In some aspects, the second domain can be positioned at the N-terminus and the first domain can be positioned at the C-terminus of the recombinant polypeptide.

In some aspects, the recombinant polypeptides disclosed herein can further comprise one or more spacer sequences (e.g., glycine residues) that can be inserted between, for example, a polypeptide sequence and the linker sequence or the polypeptide sequence and the label. The number of spacers can be adjusted according to the design and configuration of the recombinant polypeptide. The spacers can serve to provide ample space to accommodate any of the components of the recombinant polypeptide. Spacers can be one or more glycines or serines or a combination thereof.

Labels. The recombinant polypeptides described herein can further comprise one or more labels or detection tags (e.g., FLAG™ tag, epitope or protein tags, such as myc tag, 6 His, and fluorescent fusion protein). In some aspects, the label or detection tag can be a protein purification affinity tag. In some aspects, the protein purification affinity tag can be a His-tag. In some aspects, a label (e.g., FLAG™ tag) can be fused to the recombinant polypeptide. In some aspects, the disclosed methods and compositions further comprise a fusion protein, or a polynucleotide encoding the same. In various aspects, the recombinant polypeptide can comprise at least one epitope-providing amino acid sequence (e.g., “epitope-tag”), wherein the epitope-tag is selected from i) an epitope-tag added to the N- and/or C-terminus of the recombinant polypeptide; or ii) an epitope-tag inserted into a region of the recombinant polypeptide, and an epitope-tag replacing a number of amino acids in the recombinant polypeptide.

Epitope tags are short stretches of amino acids to which a specific antibody can be raised, which in some aspects allows one to specifically identify and track the tagged protein that has been added to a living organism or to cultured cells. Detection of the tagged molecule can be achieved using a number of different techniques. Examples of such techniques include: immunohistochemistry, immunoprecipitation, flow cytometry, immunofluorescence microscopy, ELISA, immunoblotting (“Western blotting”), and affinity chromatography. Epitope tags add a known epitope (e.g., antibody binding site) on the subject protein, to provide binding of a known and often high-affinity antibody, and thereby allowing one to specifically identify and track the tagged protein that has been added to a living organism or to cultured cells. Examples of epitope tags include, but are not limited to, myc, T7, GST, GFP, HA (hemagglutinin), V5 and FLAG tags. The first four examples are epitopes derived from existing molecules. In contrast, FLAG is a synthetic epitope tag designed for high antigenicity (see, e.g., U.S. Pat. Nos. 4,703,004 and 4,851,341). Epitope tags can have one or more additional functions, beyond recognition by an antibody.

In some aspects, the disclosed methods and compositions comprise an epitope-tag wherein the epitope-tag has a length of between 6 to 15 amino acids. In some aspects, the epitope-tag has a length of 9 to 11 amino acids. The disclosed methods and compositions can also comprise a recombinant polypeptide comprising two or more epitope-tags, either spaced apart or directly in tandem. Further, the disclosed methods and composition can comprise 2, 3, 4, 5 or even more epitope-tags, as long as the fusion protein maintains its biological or desired activity/activities (e.g., “functional”).

In some aspects, label, detection tag, epitope-tag, affinity tag or protein purification affinity tag can be His-tag, a FLAG-tag, a HA (hemagglutinin)-tag, a Strep-tag, a C9-tag, a glutathione S-transferase tag, a maltose-binding protein tag, a T7 tag, a V5 tag, an S tag, a SUMO tag, a TAP tag, a TRX tag, a calmodulin binding peptide, a chitin binding domain, a E2 epitope, a HaloTag, a HSV tag, a HBH tag, a KT3 tag, VSV-G tag, CD tag, Avitag, or GFP-tag or a myc-tag. The sequences of these tags are described in the literature and well known to the person of skill in art.

In some aspects, the recombinant polypeptide can be purified with an affinity capture column through binding to the label (e.g., protein purification tag). In some aspects, the expressed recombinant polypeptide can be purified using a using a fast protein liquid chromatography. The purified recombinant polypeptide can be then lyophilized and stored at −80° C. until use.

As described herein, the term “immunologically binding” is a non-covalent form of attachment between an epitope of an antigen (e.g., the epitope-tag) and the antigen-specific part of an antibody or fragment thereof. Antibodies are preferably monoclonal and must be specific for the respective epitope tag(s) as used. Antibodies include murine, human and humanized antibodies. Antibody fragments are known to the person of skill and include, amongst others, single chain Fv antibody fragments (scFv fragments) and Fab-fragments. The antibodies can be produced by regular hybridoma and/or other recombinant techniques. Many antibodies are commercially available.

The construction of recombinant polypeptides and fusion proteins from domains of known proteins, or from whole proteins or proteins and peptides, is well known. Generally, a nucleic acid molecule that encodes the desired protein and/or peptide portions are joined using genetic engineering techniques to create a single, operably linked fusion oligonucleotide. Appropriate molecular biological techniques can be found in Sambrook et al. (Molecular Cloning: A laboratory manual Second Edition Cold Spring Harbor Laboratory Press, Cold spring harbor, NY, USA, 1989). Examples of genetically engineered multi-domain proteins, including those joined by various linkers, and those containing peptide tags, can be found in the following patent documents: U.S. Pat. No. 5,994,104 (“Interleukin-12 fusion protein”); U.S. Pat. No. 5,981,177 (“Protein fusion method and construction”); U.S. Pat. No. 5,914,254 (“Expression of fusion polypeptides transported out of the cytoplasm without leader sequences”); U.S. Pat. No. 5,856,456 (“Linker for linked fusion polypeptides”); U.S. Pat. No. 5,767,260 (“Antigen-binding fusion proteins”); U.S. Pat. No. 5,696,237 (“Recombinant antibody-toxin fusion protein”); U.S. Pat. No. 5,587,455 (“Cytotoxic agent against specific virus infection”); U.S. Pat. No. 4,851,341 (“Immunoaffinity purification system”); U.S. Pat. No. 4,703,004 (“Synthesis of protein with an identification peptide”); and WO 98/36087 (“Immunological tolerance to HIV epitopes”).

The placement of the functionalizing peptide portion (epitope-tag) within the recombinant polypeptide or fusion proteins can be influenced by the activity of the functionalizing peptide portion and the need to maintain at least substantial fusion protein, such as TCR, biological activity in the fusion. Two methods for placement of a functionalizing peptide are: N-terminal, and at a location within a protein portion that exhibits amenability to insertions. Though these are not the only locations in which functionalizing peptides can be inserted, they serve as good examples, and will be used as illustrations. Other appropriate insertion locations can be identified by inserting test peptide encoding sequences (e.g., a sequence encoding the FLAG peptide) into a construct at different locations, then assaying the resultant fusion for the appropriate biological activity and functionalizing peptide activity, using assays that are appropriate for the specific portions used to construct the fusion. The activity of the subject proteins can be measured using any of various known techniques, including those described herein.

Methods of Detecting

Disclosed herein, are methods for detecting one or more anti-coronavirus antibodies in a sample. Also disclosed herein, are methods of detecting one or more antibodies against the viral antigens of coronaviruses in a sample. Further, disclosed herein, are methods of detecting one or more coronavirus antigens or one or more coronavirus virions in a sample. In some aspects, the methods can comprise a) incubating the sample with any of recombinant polypeptides disclosed herein. In some aspects, the recombinant polypeptides can comprise a first domain, wherein the first domain comprises an epitope of a coronavirus; a linker; and a second domain, wherein the second domain is a moiety that is capable of specifically binding an antigen on the surface of a red blood cell. In some aspects, the recombinant polypeptides can comprise a first domain, wherein the first domain is a moiety that is capable of specifically binding a coronavirus antigen; a linker; and a second domain, wherein the second domain is a moiety that is capable of specifically binding an antigen on the surface of a red blood cell. In some aspects, the sample comprises one or more red blood cells. In some aspects, the methods can also comprise b) mixing the sample with the recombinant polypeptide, wherein the second domain is capable of specifically binding to an antigen on the surface of a red blood cell in the sample. In some aspects, the methods can further comprise c) observing or determining whether the one or more red blood cells of the sample are agglutinated. In some aspects, the observation or determination that the one or more red blood cells of the sample are agglutinated is via cross-linking of the one or more red blood cells to the second domain of the recombinant polypeptide. In some aspects, said cross-linking of the one or more red blood cells to the second domain of the recombinant polypeptide is a result of the one or more red blood cells being induced by the binding of the first domain of the recombinant polypeptide to one or more of the anti-coronavirus antibodies in the sample. Thus, method described herein can be used to detect one or more anti-coronavirus antibodies in the sample. In some aspects, said cross-linking of the one or more red blood cells to the second domain of the recombinant polypeptide is a result of the one or more red blood cells being induced by the binding of the first domain of the recombinant polypeptide to one or more coronavirus antigens or one or more coronavirus virions in the sample. In some aspects, the method described herein can be used to detect one or more coronavirus antigens or one or more coronavirus virions in a sample. In some aspects, the time from the incubating step to agglutination can be about 1 minutes, 2 minutes, 3 minutes, 4 minutes, 5 minutes or more or any time in between.

In some aspects, the methods disclosed herein can comprise incubating and mixing a whole blood sample with the recombinant polypeptide adding fusion protein to patient whole blood, or alternatively, incubating and mixing the recombinant polypeptide to reagent red blood cells and the serum sample or plasma sample. For the latter scenario, the three substances may be incubated and mixed at the same time, or the recombinant polypeptide can be premixed with reagent red blood cells, or the recombinant polypeptide can be premixed with the serum sample or plasma sample. The mixing step can be by manual force to facilitate agglutination, which can occur, for example, on a slide and visualized with the naked eye after several minutes. In some aspects, the sample can be centrifuged to facilitate clump visualization as is performed in traditional tube testing. This process can take place in commercially available automated machines. When utilized on automated machines, the reaction may proceed with solid-phase technology, wherein, for example, the recombinant polypeptide can be present on the surface of a plate, and the red blood cells can be dispersed across the plate when agglutination occurs. For gel-based methods, the red blood cells and the plasma sample and the recombinant polypeptide can be tested in a chamber at the top of the column and incubated, followed by centrifugation to try to force the red blood cells through the gel to the bottom of the column. Red blood cells that are agglutinated will be stopped earlier in the gel than those that are not agglutinated. The gel can also contain anti-IgG, which binds to the IgG coating red blood cells in positive reactions, and further inhibits transport of the red blood cells through the gel.

In some aspects, the methods disclosed herein can further comprise scoring the red blood cell agglutination. Red blood cell agglutination can be scored according to traditional blood bank guidelines (0 to 4+), and can be done by humans or camera and image processing technologies. Beyond traditional bench tube determination of agglutination or slide assay for agglutination, other methods such as gel card testing, dry card testing, or solid-phase testing can be utilized.

In some aspects, the recombinant polypeptide can further comprise one or more labels or detection tags. In some aspects, the methods disclosed herein can further comprise mixing the sample with the recombinant polypeptide in step b) in the presence of an antibody specific to the label, thereby yielding agglutination. In some aspects, the methods disclosed herein can further comprise mixing the sample with the recombinant polypeptide in step b) in the presence of an antibody capable of specifically binding to the epitope of the coronavirus of the first domain of the recombinant polypeptide, thereby yielding agglutination. In some aspects, the methods disclosed herein can further comprise mixing the sample with the recombinant polypeptide in step b) in the presence of an antibody capable of specifically binding to the sequence or moiety that is capable of specifically binding to an antigen on the surface of the one or more red blood cells of the second domain of the recombinant polypeptide, thereby yielding agglutination. In some aspects, the antibody can be a monoclonal antibody. In some aspects, the antibody can be a polyclonal antibody.

In some aspects, the methods disclosed herein can further comprise determining coronavirus antibody titers. In some aspects, a serial dilution can be performed on a sample prior to the incubation step. In some aspects, the antibody levels or titer can be determined based on the maximal dilution that still results in agglutination. In some aspects, serial dilutions of a sample (e.g., whole blood, plasma or serum) can be performed in a 1:1 fashion (1:1, 1:2: 1:4, 1:8, 1:16, 1:32, etc.). Titer is determined by the maximal dilution that still yields agglutination. For example, serial dilution of control monoclonal antibody against a viral coronavirus antibody, such as CR3022 antibody, can serve as a control for assay validation.

In some aspects, the method can further comprise a negative control. In some aspects, the negative control can comprise a sample from a subject not exposed to a coronavirus. In some aspects, the negative control can be performed to rule out that the sample alloantibody induced agglutination. In some aspects, the method can further comprise mixing the recombinant polypeptide with a second sample. In some aspects, the second sample can be from a subject not exposed to a coronavirus.

In some aspects, the method can further comprise a positive control. In some aspects, the positive control can comprise mixing the recombinant polypeptide and the sample in the presence of antibody that is capable of specifically binding to the first domain (e.g., a viral antigen). In some aspects, the binding of the antibody to the first domain results in agglutination of red blood cells. For example, an antibody (e.g., SARS-CoV-2 monoclonal or polyclonal antibody—CR3022 antibody, ACE2-Fc protein) against the first domain comprising a coronavirus antigen (SARS-CoV-2 RBD domain). In some aspects, the positive control can comprise mixing the recombinant polypeptide and the sample in the presence of an antibody that is capable of specifically binding to the first domain, wherein the first domain is a moiety that is capable of specifically binding a coronavirus antigen. In some aspects, the positive control can comprise mixing the recombinant polypeptide and the sample in the presence of antibody that is capable of specifically binding to a label. In some aspects, the binding of the antibody to the label results in agglutination of red blood cells. In some aspects, the label can be detected by mixing the sample with an antibody that is capable of specifically binding to the label thereby detecting agglutination by the binding of the antibody to the label. For example, an anti-His tag antibody-mediated agglutination can be used as a positive control to validate the methods disclosed herein.

In some aspects, the sample can be whole blood. In some aspects, the sample can be serum or plasma. In some aspects, heterologous red blood cells can be added to the serum sample or plasma sample. In some aspects, autologous red blood cells can be added to the serum or plasma sample. In some aspects, heterologous red blood cells or autologous red blood cells can be added to the serum or plasma sample if the plasma or serum sample is lacking heterologous red blood cells or autologous red blood cells. In some aspects, the sample can be viral transport media. In some aspects, the viral transport media can be generated from a nasopharyngeal or oropharyngeal swab. In some aspects, the sample can be nasopharyngeal or oropharyngeal aspirate, respiratory secretions, sputum, or bronchalveolar lavage fluid. In some aspects, the sample can be about 20 μL, 25 μL, 30 μL, 50 μL, 100 μL, 200 μL or more, or any amount in between. In some aspects, the sample can be obtained from a subject via a finger-stick. In some aspects, the amount of the whole blood sample can be about a drop of whole blood obtain via a finger-stick. For example, about 20 μL of a red blood cell solution can be mixed with about 10 μL of an undiluted serum sample along with about 10 μL of any of the recombinant polypeptides disclosed herein in a well.

In some aspects, the time of the incubating and mixing steps can be about 1 minute, 2 minutes, 3 minutes, 4 minutes, 5 minutes or more or any amount of time in between.

In some aspects, the mixing and/or observing step can be performed in a plate well (e.g., a 96-well plate), on a slide, in a test tube, on a gel card, a dry card, a microfluidic chip or by an automated machine. In some aspects, the observing and determining whether the one or more red blood cells of the sample are agglutinated can be performed visually, for example, by the naked eye. In some aspects, a recombinant polypeptide as described herein can be added to a subject's blood, and agglutination can be detected after mixing the blood on a card. In some aspects, a recombinant polypeptide as described herein can be present on the card, and the drop of subject's blood can be added to the card, followed by solubilization of the fusion protein and agglutination can be detected after mixing.

In some aspects, the sample can be from a subject exposed to or suspected of being exposed to a coronavirus. In some aspects, the sample can be from a subject not exposed to or not suspected of being exposed to a coronavirus. In some aspects, the subject can be a human. In some aspects, the coronavirus can be severe acute respiratory syndrome coronavirus 2 (SARS-CoV-2), severe acute respiratory syndrome (SARS), middle east respiratory syndrome (MERS), human coronavirus 229E, human coronavirus NL63, Miniopterus bat coronavirus 1, Miniopterus bat coronavirus HKU8, porcine epidemic diarrhea virus, Rhinolophus bat coronavirus HKU2, Scotophilus bat coronavirus 512, bovine coronavirus, human coronavirus OC43, human coronavirus HKU1, murine coronavirus, Pipistrellus bat coronavirus HKU5, Rousettus bat coronavirus HKU9, Tylonycteris bat coronavirus HKU4, hedgehog coronavirus 1, infectious bronchitis virus, beluga whale coronavirus SW1, infectious bronchitis virus, Bulbul coronavirus HKU11, pangolin coronavirus, porcine coronavirus HKU15, WIV1-CoV, SHC014-CoV, bat-SL-CoVZC45, bat-SLCoVZXC21, SARS-CoVGZ02, BtKY72, WIV16, Rs4231, Rs7327, Rs9401, BtRs-BetaCoV/YN2018R, BtRs-BetaCoV/YN2013, Anlong-112, Rf2092, BtRs-BetaCoV/YN2018C, As6526, Rs4247, BtRs-BetaCoV/GX2013, Yunnan2011, BtRl-BetaCoV/SC2018, Shannxi2011, BtRs-BetaCoV/HuB2013, Bat_CoV_279/2005, HKU3-12, HKU3-3, HKU3-7, Longquan-140, or RaTG13.

Also disclosed herein are methods of detecting antibodies to variants of SARS-CoV-2. In some aspects, the methods disclosed herein can detect antibodies to coronavirus spike proteins and specifically receptor binding domains (RBD) of SARS-CoV-2 that develop overtime with mutations. It is thought that these mutations develop overtime to evade immune responses and specifically antibody responses. Given that vaccines are designed using spike proteins corresponding to the original sequence of SARS-CoV-2, it is a question whether the antibodies generated in response to a vaccine could still efficaciously bind to variant RBD domains. It has been shown that monoclonal antibodies designed against the RBD lose efficacy with mutations in the RBD sequence, and based on ELISA assays, that less binding strength to the RBD is noted. In some aspects, the methods described herein can be adapted to accommodate and detect changes in the SARS-CoV-2 antibodies generated in response to variants of SARS-CoV-2. For example, fusion proteins or recombinant polypeptides can be designed to incorporate these variant RBD domains. In some aspects, the canonical and variant RBD fusion proteins can be mixed together to determine if any antibodies are present that can bind to either the original or variant RBD proteins. In some aspects, it may be desirable to separate the two types of RBD fusion proteins into two separate tests, such that one test can assess whether an individual's antibody(ies) successfully binds to both RBD variant proteins. In some aspects, the strength of the agglutination can be evaluated, and such that the relative difference in binding agglutination strength between the two RBD fusion protein reactions can demonstrate differing anybody levels and/or anybody potency for an individual. A decrease in agglutination strength indicates a decreased ability for the individual's antibodies to bind to variant RBD proteins. Such a result may indicate a clinically more susceptible rate of re-infection for a vaccinated individual, or a previously coronavirus infected individual. Examples of variant RBD sequences that could be added are provided in SEQ ID NOs: 36 and 37.

Methods of Semi-Quantitative Determination of Antibody Levels or Binding Potency

Disclosed herein are methods of scoring agglutination. In some aspects, the agglutination score can be used to determine the antibody level detected in a sample. Also, disclosed herein are methods using the specific RBD agglutination score to determine the neutralizing antibody titer.

Disclosed herein are methods determining antibody levels or binding potency in a sample. In some aspects, the methods can comprise a) incubating the sample with a recombinant polypeptide comprising a first domain, wherein the first domain comprises an epitope of a coronavirus; a linker; and a second domain, wherein the second domain is a moiety that is capable of specifically binding an antigen on the surface of a red blood cell, wherein the sample comprises one or more red blood cells; b) mixing the sample with the recombinant polypeptide, wherein the second domain is capable of binding to the one or more red blood cells in the sample; c) observing or determining whether the one or more red blood cells of the sample are agglutinated; thereby detecting one or more anti-coronavirus antibodies in the sample; and d) determining an agglutination score for the sample, wherein the agglutination score indicates the antibody levels or binding potency in a sample. In some aspects, an agglutination score of 0 or 1 are deemed negative, wherein almost no red blood cell clumping occurs. In some aspects, an agglutination score of 2, 3 or 4 can indicate an increased detection of antibody levels or binding potency, respectively, in the sample. For example, an agglutination score of 4 will show a stronger agglutination (e.g., observed by large red blood cell clumps with minimal unagglutinated red blood cells) and thus indicate a higher detection of antibody levels or binding potency than an agglutination score of 3.5. An agglutination score of 4 indicates a higher detection of antibody levels or binding potency than an agglutination score of 3.5. An agglutination score of 3.5 indicates a higher detection of antibody levels or binding potency than an agglutination score of 3.0. An agglutination score of 3 indicates a higher detection of antibody levels or binding potency than an agglutination score of 2.5. An agglutination score of 2.5 indicates a higher detection of antibody levels or binding potency than an agglutination score of 2.

Further, the compositions and methods disclosed herein can be used to assess agglutination strength and antibody levels in a sample. In some aspects, the methods comprise the step of visually inspecting and assessing an agglutination reaction on a slide or a dry card to determine the strength of agglutination. In some aspects, stronger agglutinations form larger red blood cell clumps distributed across the surface indicating a higher antibody concentration. In some aspects, weaker agglutinations will form smaller red blood cell clumps indicating a lower antibody concentration. In some aspects, the methods comprise the step of visually inspecting and assessing an agglutination reaction on a gel card to determine the strength of agglutination. In some aspects, stronger agglutinations remain at the top of the gel column. In some aspects, intermediate reactions (e.g., agglutinations) will be present in the middle of the gel column. In some aspects, no reaction (e.g., agglutination) will be at the bottom of the gel column. Stronger reactions or agglutinations can indicate a higher antibody concentration. In some aspects, the methods comprise the step of visually inspecting and assessing an agglutination reaction in a test tube to determine the strength of agglutination. In some aspects, stronger agglutinations will remain as a focal red blood cell clump. In some aspects, intermediate reactions (e.g., agglutinations) will be present in small clumps across the test tube. In some aspects, no reaction (e.g., agglutination) will have red blood cells dissolve back into solution. Stronger reactions or agglutinations indicate a higher antibody concentration. In some aspects, the methods comprise the step of visually inspecting and assessing an agglutination reaction in a solid phase well to determine the strength of agglutination. In some aspects, stronger agglutinations will diffusely spread across the bottom of the well. In some aspects, intermediate reactions (e.g., agglutinations) will be a smaller focus toward the center of the well. In some aspects, no reaction (e.g., agglutination) will have red blood cells located at the center of the well. Stronger reactions or agglutinations indicate a higher antibody concentration.

In some aspects, the first domain of the recombinant polypeptide can comprise a sequence from a coronavirus spike protein. In some aspects, the sequence from the coronavirus spike protein can be the receptor binding domain of the coronavirus spike protein. In some aspects, wherein the sequence from the coronavirus spike protein can be the receptor binding domain of the coronavirus spike protein, the agglutination strength indicates the neutralizing antibody titer against the coronavirus.

In some aspects, the sample can be whole blood. In some aspects, the sample can be a serum sample or a plasma sample. In some aspects, heterologous red blood cells can be added to the serum sample or plasma sample. In some aspects, the sample can be from a subject exposed to or suspected of being exposed to a coronavirus.

In some aspects, the epitope of a coronavirus can be an epitope of a severe acute respiratory syndrome coronavirus 2 (SARS-CoV-2), a severe acute respiratory syndrome (SARS), middle east respiratory syndrome (MERS), a human coronavirus 229E, a human coronavirus NL63, Miniopterus bat coronavirus 1, a Miniopterus bat coronavirus HKU8, porcine epidemic diarrhea virus, a Rhinolophus bat coronavirus HKU2, a Scotophilus bat coronavirus 512, a bovine coronavirus, a human coronavirus OC43, a human coronavirus HKU1, murine coronavirus, a Pipistrellus bat coronavirus HKU5, a Rousettus bat coronavirus HKU9, a Tylonycteris bat coronavirus HKU4, a hedgehog coronavirus 1, an infectious bronchitis virus, a beluga whale coronavirus SW1, an infectious bronchitis virus, a Bulbul coronavirus HKU11, a pangolin coronavirus, a porcine coronavirus HKU15, a WIV1-CoV, a SHC014-CoV, a bat-SL-CoVZC45, a bat-SLCoVZXC21, a SARS-CoVGZ02, a BtKY72, a WIV16, Rs4231, a Rs7327, a Rs9401, a BtRs-BetaCoV/YN2018R, a BtRs-BetaCoV/YN2013, Anlong-112, a Rf2092, a BtRs-BetaCoV/YN2018C, a As6526, Rs4247, a BtRs-BetaCoV/GX2013, a Yunnan2011, a BtRl-BetaCoV/SC2018, a Shannxi2011, a BtRs-BetaCoV/HuB2013, a Bat_CoV_279/2005, a HKU3-12, a HKU3-3, a HKU3-7, a Longquan-140, or a RaTG13 or a variant thereof.

Methods for Determining Virus Variants

Disclosed herein are methods identifying a difference in antibody levels or binding strength to one or more SAR-CoV-2 proteins in a sample. In some aspects, the method can comprise performing two separate agglutination reactions either sequentially or simultaneously. In some aspects, the first agglutination reaction can be used to determine the antibody level or binding strength to a SAR-CoV-2 variant protein in a sample. In some aspects, the second agglutination reaction can be used to determine the antibody level or binding strength to a canonical SAR-CoV-2 protein in a sample. In some aspects, the method can comprise determining the relative agglutination strength in the first and second agglutination reactions, comparing the relative agglutination strength between the first and second agglutination reactions, and identifying a difference in the relative agglutination strength between the first and second agglutination reactions.

Disclosed herein are methods of improving sensitivity for an agglutination assay. In some aspects, the methods can use two or more recombinant polypeptides. In some aspects, one or more of the recombinant polypeptides can comprise a first domain, wherein the first domain comprises an epitope of a variant coronavirus, and thus, the methods can generate a readout that comprises information relating to antibody binding to any coronavirus variant. In some aspects, the methods can comprise a) incubating a sample with two or more recombinant polypeptides, wherein a first recombinant polypeptide comprising a first domain, wherein the first domain comprises an epitope of a variant coronavirus; a linker; and a second domain, wherein the second domain is a moiety that is capable of specifically binding an antigen on the surface of a red blood cell; wherein a second recombinant polypeptide comprising a first domain, wherein the first domain comprises an epitope of a canonical coronavirus; a linker; and a second domain, wherein the second domain is a moiety that is capable of specifically binding an antigen on the surface of a red blood cell; wherein the sample comprises one or more red blood cells; b) mixing the sample with the first and second recombinant polypeptides, wherein the second domain of the first and second recombinant polypeptides is capable of binding to the one or more red blood cells in the sample; and c) observing or determining whether the one or more red blood cells of the sample are agglutinated; thereby detecting one or more anti-coronavirus antibodies in the sample. In some aspects, the sample can bind to both of the second domains of the first and second recombinant polypeptides. In some aspects, the sample can bind to the second domain of the first recombinant polypeptide. In some aspects, the sample can bind to the second domain of the second recombinant polypeptide.

Kits

Disclosed herein are kits comprising one or more of the disclosed recombinant polypeptides. Disclosed herein are kits comprising recombinant polypeptide comprising: a first domain comprising an epitope of a coronavirus; a linker; and a second domain, wherein the second domain is a moiety that is capable of specifically binding an antigen on the surface of a red blood cell. In some aspects, the recombinant polypeptides can comprise a first domain, wherein the first domain is a moiety that is capable of specifically binding a coronavirus antigen; a linker; and a second domain, wherein the second domain is a moiety that is capable of specifically binding an antigen on the surface of a red blood cell. In some aspects, the disclosed kits can comprise instructions for preparing the recombinant polypeptide and performing the method of detecting one or more anti-coronavirus antibodies in a sample.

In some aspects, the kits can comprise recombinant polypeptides wherein the first domain is a sequence from the coronavirus spike (S) protein, envelope (E) protein, a membrane (M) protein, a nucleocapsid (N) protein or an antigenic fragment thereof and the second domain is a single chain variable fragment (scFv) specific for an antigen on the surface of one or more red blood cells. In some aspects, the kits can comprise recombinant polypeptides wherein the first domain is a capable of specifically binding a coronavirus antigen and the second domain is a single chain variable fragment (scFv) specific for an antigen on the surface of one or more red blood cells. In other aspects, the kits can further comprise a first linker between the first domain and the second domain. In some aspects, the linker can be a peptide. In some aspects, the kits can further comprise a label or detectable tag. In some aspects, the kits can also comprise nucleic acids comprising the recombinant polypeptides, vectors and/or cells as described herein.

### EXAMPLES

### Example 1: A Method for Detecting Antibodies Against SARS-CoV-2

Design, Construction and Validation of Hemagglutination-inducing fusion proteins. Design of fusion proteins: Fusion proteins will be designed to tag the RBC surface with SARS-CoV-2 spike protein. A combination of different viral antigens will be tested (S1 domain of Spike, S2 domain of Spike, Receptor Binding Domain (RBD) of Spike, and Nucleocapsid) that have shown high level induction of antibodies in SARS patients. Similarly, several different antibodies against red blood cell antigens such as H antigen (carbohydrate in ABO) and Glycophorin A will be tested in order to ascertain which antigen has the best properties for RBC binding and agglutination. Data with fusion protein targeting H antigen and RBD is described herein.

Validation of fusion protein function: Fusion protein expression will be verified by Western blot, and binding to the surface of red blood cells will be verified by flow cytometry with secondary ant-His tag antibody. Anti-His tag antibody will also be used as a control for agglutination. Stability of the fusion proteins in solution and in different temperatures over time, important for global health uses.

Define assay parameters for efficient red blood cell agglutination in presence of COVID-19 patient serum. Testing agglutination of RBCs in presence of patient serum. Different amounts of fusion protein will be tested, along with different viral antigens, to determine, which antigen is at the highest levels in patients, as well as determining which combination of antigens can be used to induce agglutination. The results described herein indicates successful agglutination with a SARS-CoV2 RBD-scFv fusion protein. The fusion protein can be re-designed to improve potency.

Developing agglutination test for use in different formats: The methods will be developed as a point of care slide agglutination test with an optimized mixing time. The methods will also be tested for, tube and gel testing blood bank assays. The ability of automated hemagglutination testing systems to be repurposed will also be explored. The performance of assay for manual and automated antibody titration testing will be evaluated.

Correlate test with ELISA and lateral flow assays: The methods described herein will be compared with other serological tests on the same clinical samples. Antibody titers will also be compared with in vitro neutralization assay results.

The clinical profile of hemagglutination-based SARS-CoV-2 serology test will also be defined. For example, the assay will be tested in blinded COVID19 patients and negative patient samples to determine sensitivity and specificity for regulatory approval. Ease of use will also be evaluated, by teaching healthcare and non-healthcare providers how to perform the assay to assess performance in real world conditions.

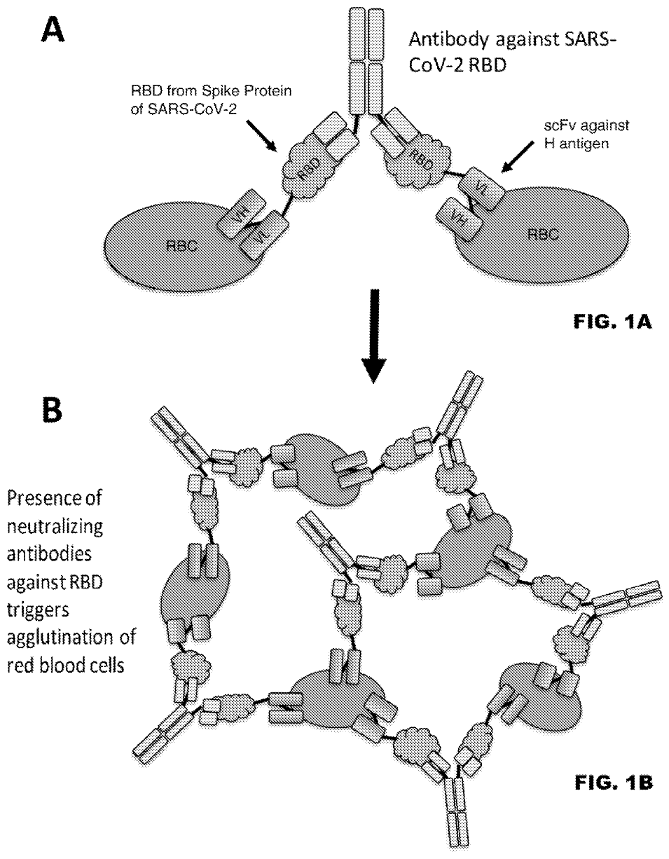

Design and Construction of fusion protein. Fusion proteins will be constructed with viral antigen and a single chain variable fragment (scFv) against a red blood cell antigen. To this end, a receptor binding domain (RBD)-scFv fusion protein was designed as depicted in FIG. 1A to decorate SARS-CoV-2 antigens on the surface of RBCs. These decorated RBCs would then yield hemagglutination in the presence of SARS-CoV-2 antibodies (FIG. 1). The RBD of the SARS-CoV-2 spike protein, corresponding to amino acids 330-524 of the spike protein (Wrapp, D. et al. Science 367, 1260-1263 (2020)), was chosen for its small size and stable folding, as well as the fact that the RBD is the target of the majority of neutralizing antibodies against coronaviruses (Tai, W. et al. Cellular and Molecular Immunology 7, 226-8 (2020)). Any positive test for antibodies binding to RBD would be highly suggestive of the presence of neutralizing antibodies that would be protective of reinfection (Sui, J. et al. Journal of Virology 88, 13769-13780 (2014)).

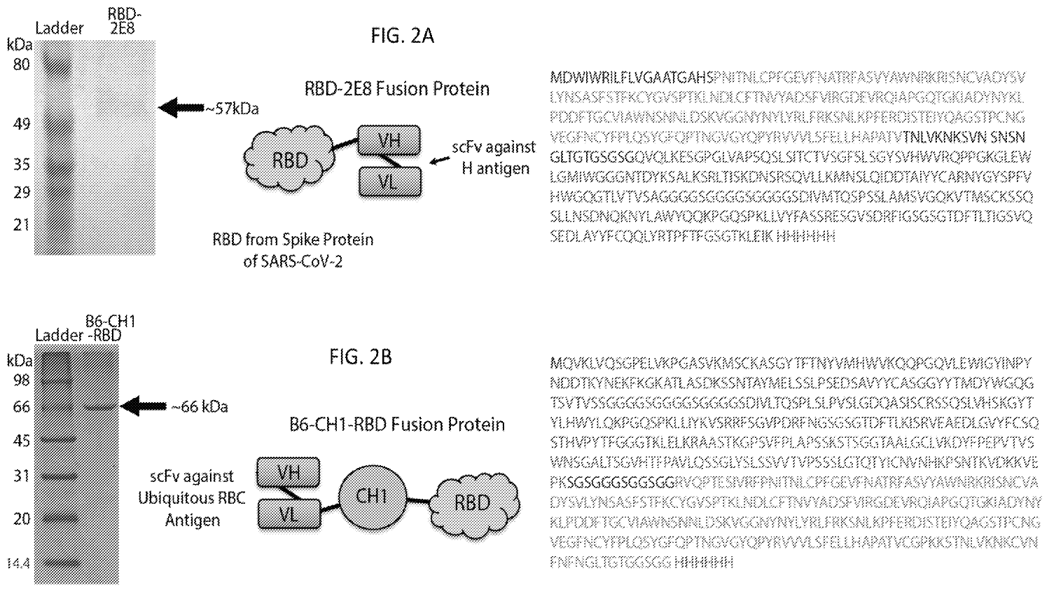

The scFv was connected to the RBD that targets the H antigen, a carbohydrate antigen located within the ABO polysaccharides (Scharberg, E. A., Olsen, C. & Bugert, P. The H blood group system. Immunohematology 32, 112-118 (2016)). The H antigen is ubiquitous on RBCs in the human population, except among Bombay donors (Shrivastava, M., et al. Asian J Transfus Sci 9, 74-77 (2015)). A previous study indicated that the scFv 2E8 could successfully bind to RBCs and be used to display HIV gp41 in an assay to detect HIV antibodies in a similar RBC agglutination assay (Shao, C. & Zhang, J. Cellular and Molecular Immunology 5, 299-306 (2008)). The RBD-scFv also contained an IgG heavy chain secretion signal to allow export from mammalian cells, and a hexa-histidine tag located at the end of the protein to allow for convenient purification. The RBD-scFv was synthesized (Twist Bioscience) and cloned into pIRII-IRES-GFP²⁴ to form pIRII-RBD-scFv-IRES-GFP. The RBD-scFv fusion protein was collected from cell culture supernatant after the transfection of expression plasmids in 293T cells. After purification with a nickel column via His-tag affinity, the RBD-scFv protein was run on a protein gel to confirm the proper size (FIG. 2A).

Beyond the initial testing of RBD-scFv, additional combinations of scFv's against human RBC antigens and SARS-CoV-2 viral antigens will be designed and tested as fusion proteins. The spike protein is a large structure with S1 domain forming a crown, and the S2 domain forming a stalk. The two domains can be explored separately or together in a fusion protein format to encompass the epitopes antibodies against the spike protein may target (Li, F., et al. Science 309, 1864-1868 (2005)). The SARS-CoV-2 nucleocapsid protein is also a frequent target of antibodies in patients and will be tested as well. On the RBC antigen front, the antibody 10F7 targeting Glycophorin A and the B6 antibody targets a high frequency antigen across humans will be evaluated (Gupta, A. & Chaudhary, V. K. J. Clin. Microbiol. 41, 2814-2821 (2003)). Linker regions will also be evaluated to obtain proper geometry.

Protein Production and Purification. RBD-scFv protein was produced in HEK 293T cells to preserve proper folding and glycosylation patterns of the viral domain. pIRII-RBD-scFv-IRES-GFP and pCMV-hyperPB (hyperactive piggyBac transposase) were co-transfected using Lipofectamine 3000 into HEK 293T cells. The piggyBac transposon system afforded stable integration and protein production. The supernatant containing RBD-scFv was collected at 72 hours. RBD-scFv was purified from the supernatant using Capturem™ His-Tagged Purification Miniprep Kit (Takara Bio). One prep of 800 mL supernatant through one column of the kit yielded 102 mg/mL of RBD-scFv measured by NanoDrop™ 2000/2000c Spectrophotometers (ThermoFisher), which was used in subsequent experiments. The correct size of the purified RBD-scFv protein (52.4 kDa turning into −57 kDa with glycosylation) was confirmed by protein gel electrophoresis (FIG. 2A) using Mini-PROTEAN TGX Precast Gels 4-20% (Bio-RAD). Other fusion proteins under construction will be purified in a similar manner. In some aspects, protein production will be carried out in insect, bacterial, or yeast cells which could offer similar ability for scaling.

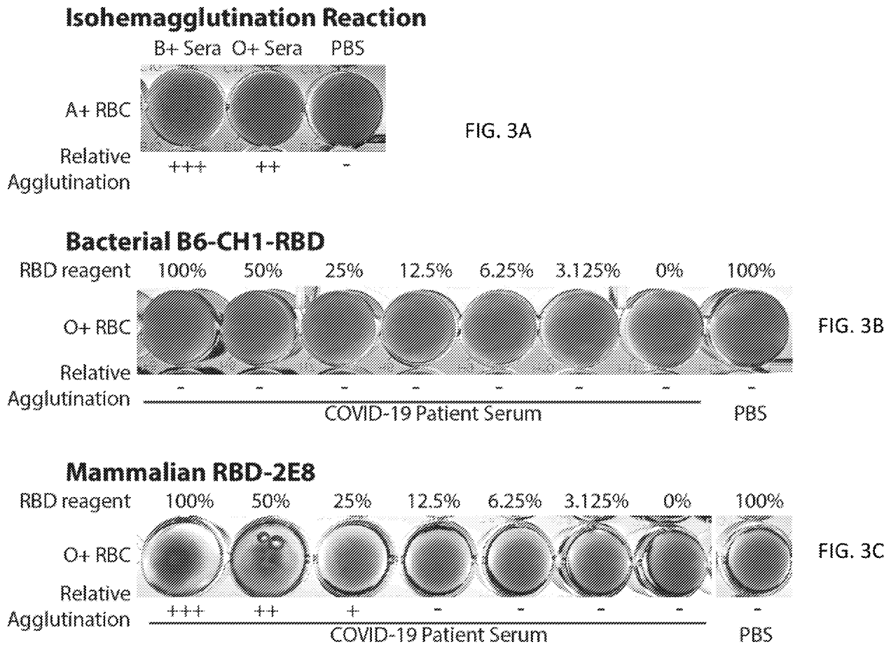

Red blood cell agglutination assay to detect SARS-CoV-2 antibodies. Agglutination testing will be carried out with the fusion proteins described herein. The following reagents were acquired for testing the RBC agglutination assay: O-type Rh-negative red blood cells suspended in 24% solution (Immucor) were obtained from the Johns Hopkins Blood Bank. A deidentified, discarded serum sample from a recovered COVID19 patient was evaluated. The patient specimen was collected greater than 28 days post symptoms of COVID-19 with negative PCR testing upon discharge. The RBD-scFv fusion protein was mixed with RBCs in the presence of COVID19 patient serum in order to detect for agglutination. The assay was carried out in a small total volume, 40 μL, in a 96-well plate. The RBC solution (2-4% red blood cells) is at a dilution commonly used in manual tube testing for ABO typing in blood banks. More specifically, a round bottom 96-well plate was used similar to validation in previous reports. Twenty μL of red blood cell solution was mixed with 10 μL of undiluted patient serum along with 10 μL of RBD-scFv solution was placed in each well, following a similar protocol from a previous study (Shao, C. & Zhang, J. Cellular and Molecular Immunology 5, 299-306 (2008)). The solution was thoroughly mixed with the pipette and allowed to incubate for five minutes at room temperature before agglutination was assessed with visualization by the naked eye. Given that the amount of RBD-scFv fusion protein needed to cross-link antibodies and RBCs and trigger agglutination as unknown, a dilution series was performed, starting with the maximal concentration of the RBD-scFv stock solution through 5 successive 1:1 dilutions. A seventh well of containing patient serum, 10 μL of phosphate-buffered saline (PBS) and RBC solution alone was used as a negative control to rule out potential patient alloantibody induced agglutination. After five minutes of incubation, agglutination was observed in the three highest concentrations of RBD-scFv protein, with no agglutination observed in the more dilute RBD-scFv concentrations (FIG. 3C). Decreasing levels of agglutination were observed in the three highest levels of RBD-scFv. Incubation of RBD-scFv and RBC's without patient serum did not yield any agglutination. These data indicate the presence of antibodies that bind to RBD in the patient's serum, with agglutination activity reflective of RBD-scFv concentration.

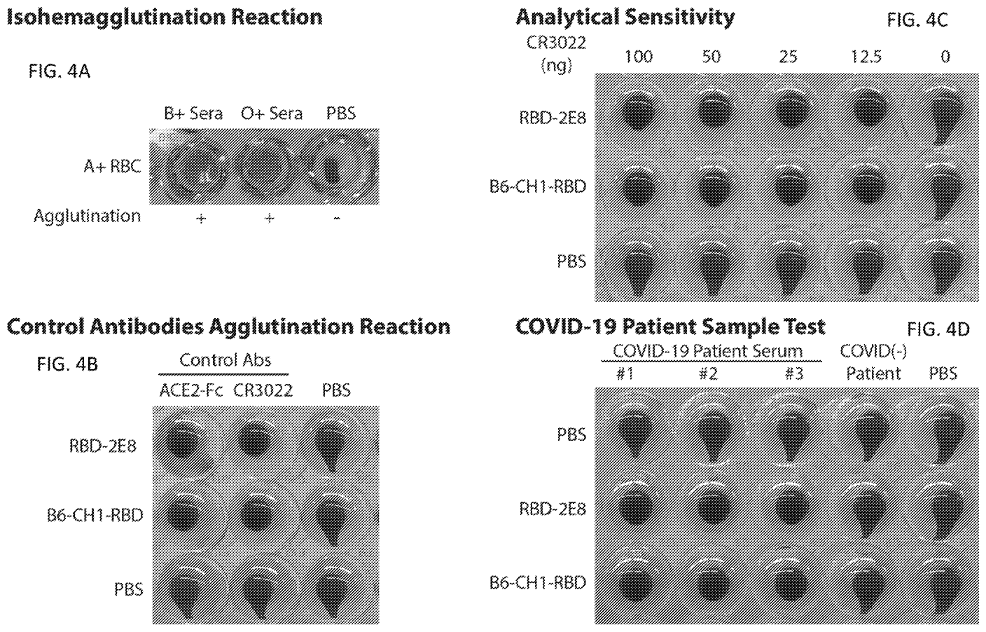

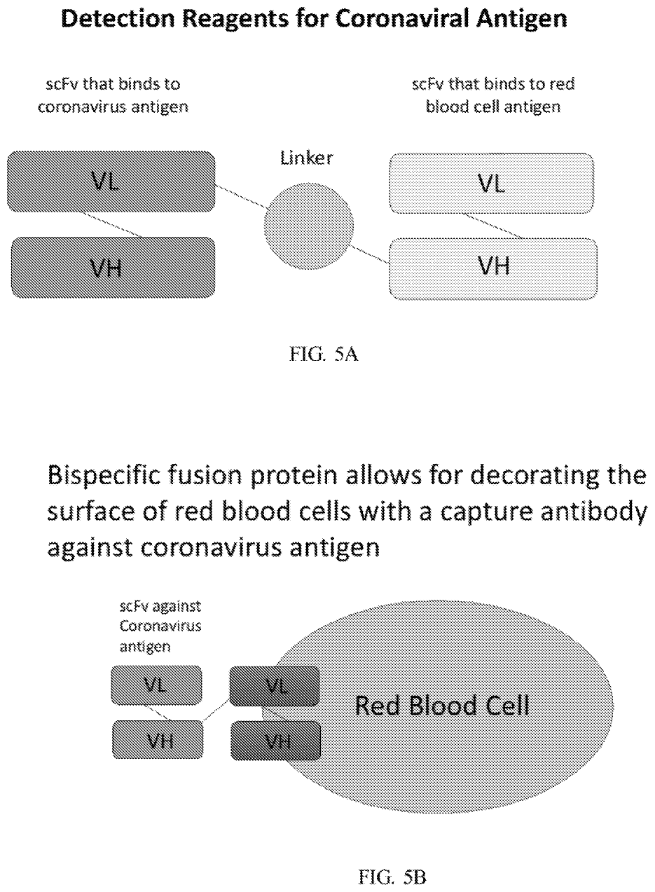

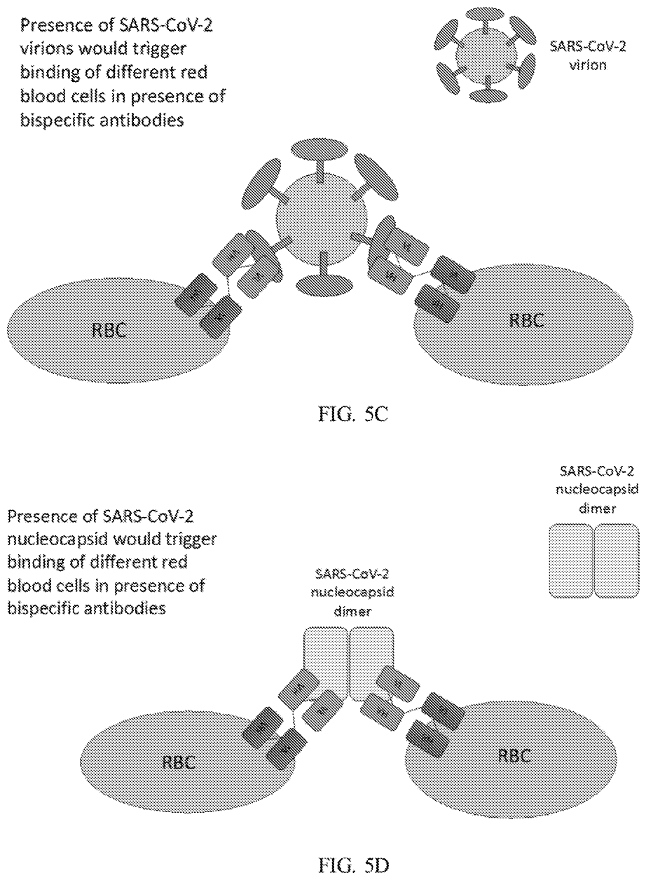

Next, the potency of additional fusion proteins covering different viral antigens will be evaluated. The relative ability of different SARS-CoV-2 antigens (e.g., S1, S2, RBD, and Nucleocapsid) to induce agglutination will be compared, as well as their synergy. The assay will also be tested in the traditional point of care slide agglutination assay (FIG. 4) that could be scaled rapidly. Adapting the assay to automated agglutination machines will also facilitate scaling as well. Titration of antibodies will also be tested by manual and automated means, which will be important for the deployment to identify the best individuals to donate convalescent plasma as a treatment for COVID-19 patients (Bloch, E. M. et al. J. Clin. Invest. (2020)).

Sensitivity and specificity testing will also be carried out. The assay can be distributed to other providers to ensure they can perform the assay and consistently get expected results with known positives and negatives. The agglutination assay will also be correlated with known results from other ELISA and lateral flow assays being developed, in order to ensure similar performance. Larger scale production of viral protein will also be explored.

The methods described herein to detect red blood cell agglutination which is visible by the naked eye to detect antibodies against SARS-CoV-2, is a cheap, and scalable method that can be deployed in a wide variety of health care settings. Given the likely need for repeated testing of millions of individuals across the world repeated in the coming year, new options such as the methods described herein are needed, particularly for low-resource countries.

Next, the potency of the agglutination observed will be evaluated and improved as well as the speed to result. These steps will involve screening additional combinations of viral antigens and antibody moieties against RBC's. Western blot will also be performed to verify successful production of fusion proteins after nickel column purification. Flow cytometry studies will be performed to evaluate binding efficiency of the fusion proteins to red blood cells, using it to estimate relative density of viral antigen on cell surface. Combinatorial strategies will also be tested by flow cytometry.

Fusion proteins will be titrated in different amounts in the presence of COVID-19 patient serum to test for agglutination efficiency and minimum concentration needed. Patient whole blood testing will also be performed to ensure assay performance in this modality as well.

Sensitivity and specificity testing will next be performed with optimized fusion protein or combination of fusion proteins formulated for the final version of the assay. Negative patient samples (e.g., 200) and positive COVID-19 patient samples (e.g., 30) will be used. A past prior patient will be identified to simulate the procedure of finger-prick blood draw, and testing on a card with result in two minutes to ensure performance of the assay in the real world. Ease of use can also be evaluated, by teaching healthcare and non-healthcare providers how to perform the assay to assess performance in real world conditions.

Next, protein production will be scaled, for example, to provide protein for several thousand tests (1 μg per test based on pilot, so 100 mg would equal 100,000 tests).

In some aspects, blood banks can be used to evaluate the method and identify convalescent plasma donors, as well as to facilitate faster antibody titering of patient plasma.

Described herein are a rapid, point of care RBC agglutination tests for SARS-CoV-2 antibodies using a fingerstick drop of blood within two minutes. For example, 30 μL of RBCs and serum can be used. For whole blood assays, RBD-scFv fusion protein alone solution could mixed with whole blood in order to facilitate the assay. Given the costs and complexities of blood specimen processing, this represents a significant advantage. The estimated cost for the research assay using small scale production and purification was 25 cents (U.S.) per test, which should be able to be reduced under 1 cent per test with larger-scale protein production and purification. At this scale, every American could be tested for $3 million dollars. While the assay was carried out in 96 well plates similar to previous studies (Shao, C. & Zhang, J. Cellular and Molecular Immunology 5, 299-306 (2008)), the format is readily transferrable to slide agglutination test, where the components are mixed on a re-usable glass slide by stirring followed by short incubation (Gupta, A. & Chaudhary, V. K. J. Clin. Microbiol. 41, 2814-2821 (2003)). Concerning practicality, the methods and compositions described herein can be used in healthcare settings across the world. Reagent stability is important for settings with refrigeration. Past studies with similar fusion proteins for hemagglutination assays found stability at least 30 days at 37° C. and 6 months at 4° C. with no loss of assay agglutination activity (Gupta, A. & Chaudhary, V. K. J. Clin. Microbiol. 41, 2814-2821 (2003)). While the point of care assay potential is emphasized in this study for simplicity, the fusion protein reagents could potentially be added into other hemagglutination assays used in clinical labs today, such as tube testing and gel testing, as well as on automated solid-phase machines. The potential repurposing of automated hemagglutination assay machines, in particular, could allow for high-throughput testing of hundreds of thousands of tests today using existing clinical infrastructure. Of note is that the methods described herein currently does not distinguish between IgG, IgA, or IgM against SARS-CoV-2.

The compositions and methods described herein can be used to design fusion proteins to detect certain subsets of SARS-CoV-2 proteins. For example a fusion protein comprising the entire or larger portions of the ectodomain of the spike protein which patients have been reported to have much higher antibody titers against (Stadlbauer, D. et al. Curr Protoc Microbiol 57, e100 (2020)), can be used which can be useful. Moreover, peptides from the SARS-CoV-2 nucleocapsid protein can also be employed. Further testing can be carried out to reduce the incubation time by optimizing fusion protein and reagent concentrations. The sensitivity and specificity of the assay can be confirmed with more COVID-19 patient samples. For clinical applications, the important utility of the test will be the ability to rapidly detect who has been exposed and has antibodies against COVID-19 in the population. Beyond epidemiology, the ability to detect potentially neutralizing antibodies in the plasma of COVID-19 recovered patients would be useful in the continued deployment of convalescent sera or hyperimmune globulin as a therapy against COVID-19 (Kruse, R. L. Therapeutic strategies in an outbreak scenario to treat the novel coronavirus originating in Wuhan, China. F1000Res 9, 72 (2020); and Duan, K. et al. Proc. Natl. Acad. Sci. U.S.A. 14, 202004168 (2020)). Convalescent serum has shown promise against the SARS coronavirus (Cheng, Y. et al. Eur. J. Clin. Microbiol. Infect. Dis. 24, 44-46 (2005)), but studies are generally limited in efficacy by variable and low neutralizing antibody titers in donors (van Griensven, J., et al. N. Engl. J. Med. 375, 2307-2309 (2016)). The ability to more rapidly screen donors for high titer antibodies against SARS-CoV-2, then they could be selected for plasma donation and this therapy could be more widely scaled and beneficial (Bloch, E. M. et al. J. Clin. Invest. (2020). doi:10.1172/JCI138745). Monitoring antibody development in patients may also help stratify COVID-19 patients and offer prognostic value for those who may clinically improve versus suffering respiratory demise.

### Example 2: A Method for Detecting SARS-CoV-2 Antigen in Body Fluids

For the assay, respiratory fluid from the nasopharynx or the oropharynx can be mixed with red blood cells from the patient or an allogeneic donor and then added to the recombinant polypeptide. Respiratory fluid can be obtained with a swab in cases and directly mixed with red blood cells, or the swab can be dipped into fluids, and then this fluid can be mixed with the red blood cells. Finally, respiratory fluid can also be obtained from fluid from a bronchoalveolar lavage procedure.

The virions in the respiratory fluids displaying the spike protein on the surface will contact the recombinant polypeptide and mediate crosslinking of red blood cells given the very high numbers spike proteins on the viral surface. This will mediate visible agglutination after mixing and several minutes of incubation.

In some aspects, blood can be taken from a patient to detect for the presence of circulating virions in the bloodstream. In a similar way, the recombinant polypeptide can be added to whole blood or reconstituted blood from patient plasma or serum with allogeneic red blood cells that can then be mixed and allowed to agglutinate based on the presence of circulating viral antigens (e.g., coronavirus) in the blood.

In other aspects, the presence of coronaviral nucleocapsid antigen can be detected in respiratory fluids or blood for the diagnosis of infection. Coronavirus nucleocapsid has been shown to be shed from virally infected cells at high levels and be found in extracellular spaces. For this purpose, the recombinant polypeptide binds to nucleocapsid, which normally exists as a free dimer, thereby leading to one dimer displaying two different red blood cell binding moieties, similar to an antibody. The recombinant polypeptide binding to the nucleocapsid dimer would then facilitate crosslinking of red blood cells in a whole blood sample or a reconstituted blood sample, either with plasma, serum, or respiratory fluids added.

For any of the methods described herein, the method can proceed by mixing the components together and allowing incubation for several minutes, after which agglutination can be observed with the naked eye or by microscopy.

To test the methods disclosed herein, SARS-CoV-2 pseudovirion was constructed that includes SARS-CoV-2 spike protein expressed on the surface of a retroviral gag protein-induced particle, with no viral genome inside. This SARS-CoV-2 pseudovirion can be efficiently produced in cell culture after transfection. The results demonstrate the feasibility of the assay yielding agglutination in a 96-well plate upon mixing SARS-CoV-2 pseudovirions and fusion protein, similar to the serology assay also disclosed herein. In combination with the microfluidic device detailed herein, a fast point of care detection device for SARS-CoV-2, with the microfluidic chamber enhancing agglutination reactions and removing the subjectivity of agglutination interpretation by the naked eye can be provided.

### Example 3: A Rapid, Point-of-Care Red Blood Cell Agglutination Assay Detecting Antibodies Against SARS-CoV-2

The COVID-19 pandemic has caused significant morbidity and mortality. There is an urgent need for serological tests to detect antibodies against SARS-CoV-2, which could be used to assess past infection, evaluate responses to vaccines in development, and determine individuals who may be protected from future infection. Current serological tests developed for SARS-CoV-2 rely on traditional technologies such as enzyme-linked immunosorbent assays (ELISA) and lateral flow assays, which have not scaled to meet the demand of hundreds of millions of antibody tests so far. Herein, an alternative method of antibody testing is described that depends on one protein reagent being added to patient serum/plasma or whole blood with direct, visual readout. Two novel fusion proteins, RBD-2E8 (SEQ ID NO: 16) and B6-CH1-RBD (SEQ ID NO: 17), were designed to bind red blood cells (RBCs) via a single-chain variable fragment (scFv), thereby displaying the receptor-binding domain (RBD) of SARS-CoV-2 spike protein on the surface of RBCs. Mixing mammalian-derived RBD-2E8 and B6-CH1-RBD with convalescent COVID-19 patient serum and RBCs led to visible hemagglutination, indicating the presence of antibodies against SARS-CoV-2 RBD. B6-CH1-RBD made in bacteria was not as effective in inducing agglutination, indicating better recognition of RBD epitopes in mammalian cells. The methods disclosed herein can be rapidly deployed in low-resource settings at minimal cost, and use in low-resource settings for detecting SARS-CoV-2 antibodies.

Serologic testing for antibodies against SARS-CoV-2 could detect both recent and past infection, which is useful for surveillance and epidemiological studies (L. Guo, et al., Clin Infect Dis. 24 (2020) 490). However, current enzyme-linked immunosorbent assay (ELISA) tests for COVID-19 require a number of steps, washes, and reagents, involving hours of manual time and/or automated machines (N. M. A. Okba, et al., Emerging Infect. Dis. 26 (2020) 270). Lateral flow immunoassays have been developed for SARS-CoV-2, but still require the manufacturing of strips, plastic holders, and multiple different antibody types and conjugates (Z. Li, Y., et al., J. Med. Virol. (2020) jmv.25727). Thus, an urgent need exists for a low complexity assay that could be performed as a point-of-care test in low-resourced health care settings, without the need for machines.

Described herein are methods that use an RBC agglutination to detect antibodies against the receptor-binding domain (RBD) of SARS-CoV-2 spike protein in COVID-19 patients, which is the frequent target of neutralizing antibodies against coronaviruses (L. Premkumar, et al., Sci Immunol. 5 (2020)). These methods can be used in low-resource settings as a simple method of testing for current or past SARS-CoV-2 infection.

Materials and Methods. Gene construction. Two different fusion proteins were designed. The first fusion protein was SARS-CoV-2 RBD (amino acids 330-524 of the spike protein) of the SARS-CoV-2 spike protein (D. Wrapp, et al., Science. 367 (2020) 1260-1263), connected via a short linker to a single-chain variable fragment (scFv) derived from the antibody 2E8 that binds to the H antigen on RBCs (C. Shao, and J. Zhang, Cellular and Molecular Immunology. 5 (2008) 299-306) to form RBD-2E8 (FIG. 1). RBD-2E8 also contained an IgG heavy-chain secretion signal for export from mammalian cells, and a hexa-histidine tag located at C-terminus to allow for convenient purification. The RBD-2E8 gene was synthesized (Twist Bioscience) and cloned into a pCMV-IRES-GFP vector.

A second fusion protein, B6-CH1-RBD, was designed, consisting of an scFv binding to RBCs at the N-terminus, and the RBD sequence at the C-terminus with hexa-histidine tag. B6 is an scFv clone against a high frequency antigen on human RBCs (A. Gupta, et al., MAbs. 1 (2009) 268-280). The human IgG CH1 domain was included as a linker to facilitate additional length for antigen binding (G. Coia, et al., J. Immunol. Methods. 192 (1996) 13-23). RBD sequence was longer than prior, ranging from 319-550 amino acid. B6-CH1-RBD was synthesized with an IgG heavy-chain secretion signal (Twist Bioscience) and cloned into the pTwist vector driven by a CMV promoter. A second B6-CH1-RBD gene was codon-optimized for E. coli expression and synthesized by BioBasic (Markham, Ontario, Canada), and subsequently cloned into a pET vector for expression.

Protein Production and Purification. The RBD-2E8 (e.g., SEQ ID NO: 38) and B6-CH1-RBD (e.g., SEQ ID NO: 39) fusion proteins were first produced in 293T cells in order to preserve proper folding and glycosylation patterns of the viral domain. pCMV-RBD-2E8-IRES-GFP and pTwist-B6-CH1-RBD were transfected using Lipofectamine 3000 (ThermoFisher) into 293T cells. The supernatant containing RBD-2E8 and B6-CH1-RBD protein was collected at 48-72 hours. RBD-2E8 and B6-CH1-RBD was purified from the supernatant using Capturem™ His-Tagged Purification Miniprep Kit (Takara Bio). Supernatant of 800 μL purified through one column of the kit and adjusted to ˜100 μg/mL of RBD-2E8 and B6-CH1-RBD, as measured by NanoDrop™ 2000/2000c Spectrophotometers (ThermoFisher). The correct size of the purified RBD-2E8 protein (˜57 kDa with glycosylation) was confirmed by protein gel electrophoresis using Mini-PROTEAN TGX Precast Gels 4-20% (Bio-RAD) and Simply Blue Safe Stain (ThermoFisher).

B6-CH1-RBD, was also expressed in E. coli, since bacterial expression was employed for similar, previous hemagglutination reagents (A. Gupta and V. K. Chaudhary, J. Clin. Microbiol. 41 (2003) 2814-2821; and I. Habib, et al., Anal Biochem. 438 (2013) 82-89). Briefly, B6-CH1-RBD was produced in E. coli via custom production with a commercial provider (BioBasic). Protein was purified with His-column affinity as an insoluble product, and subsequently refolded. The correct size of the purified B6-CH1-RBD was ˜66 kDa and confirmed on an SDS-PAGE gel. The steps were performed by BioBasic.

Patient serum samples. De-identified, discarded serum samples were collected and provided from Johns Hopkins Bayview Hospital, representing blood draws by phlebotomists for other medical testing during hospitalization. The clinical lab had collected and banked recovered COVID-19 patient specimens who were greater than 28 days post COVID-19 symptoms with negative PCR testing at the time. Patients were previously positive by nasopharyngeal swab PCR testing at their admission for COVID-19. Aliquots of these samples were then provided for the study without identification. De-identified, discarded patient samples with known ABO typing were also provided by the hospital as control anti-sera for isohemagglutination assays.

Red blood cell agglutination testing. For the RBC agglutination assay, a round-bottom 96-well plate (CoStar) was used. 0-type Rh-positive red blood cells suspended in 24% solution (Immucor) were obtained.

The assay was carried out in two different conditions. In the first, 20 μL of RBC solution, 10 μL of undiluted COVID-19 patient serum, and 10 μL of RBD-2E8 or B6-CH1-RBD solution were pipetted into each well (C. Shao and J. Zhang, Cellular and Molecular Immunology. 5 (2008) 299-306). The solution was thoroughly mixed and incubated for 5-minutes at room temperature; agglutination was then visualized by the naked eye. For testing COVID-19 patient serum, a dilution series of RBD-2E8 was performed to test for optimal levels of protein to induce agglutination in presence of patient anti-RBD antibodies. A series of six 1:1 dilutions were performed from the ˜100 μg/mL of RBD-2E8, B6-CH1-RBD (mammalian), and B6-CH1-RBD (bacterial) stocks. A seventh well containing non-infected patient serum, 10 μL of phosphate-buffered saline (PBS) and RBC solution alone was used as a negative control to rule out potential patient alloantibody induced agglutination, or alternatively, cold-reactive IgM autoantibodies.

In a second assay condition, a longer incubation time was utilized to augment a weaker rate of hemagglutination according to a published protocol (A. Townsend, et al., A hemagglutination test for rapid detection of antibodies to SARS-CoV-2, medRxiv. (2020) 2020.10.02.20205831). Fifty μL of RBCs (2-4%) were added to 50 μL of COVID-19 patient serum along with 25 μL of purified mammalian fusion protein solution. For the bacterial protein, a solution of 10 μg/mL of B6-CH1-RBD was prepared and 50 μL added to the reaction. After mixing, the solution was allowed to incubate for one hour in the 96-well plate. To read the assay, the plate was then tilted to allow the RBC pellet to dislodge; agglutination kept the place effectively in place. In other experiments, 50 μL solution of recombinant proteins ACE2-Fc (10 μg/mL), and CR3022 antibody (0.6 μg/mL) (ProMab Biotechnologies) were added in the place of COVID-19 patient serum at the designated amounts listed in the figures, and the assay similarly executed.

Results. Construction of SARS-CoV-2 fusion proteins. SARS-CoV-2 antigens were decorated on the surface of RBCs (FIG. 1A). These decorated RBCs would then yield hemagglutination in the presence of SARS-CoV-2 antibodies (FIG. 1). FIG. 1A-B shows the mechanism of using fusion proteins to induce hemagglutination to detect SARS-CoV-2 antibodies. A fusion protein, for example, RBD-2E8, was constructed, consisting of the receptor-binding domain (RBD) of the SARS-CoV-2 spike protein at the N-terminus connected via a linker to a single-chain variable fragment (scFv, consisting of VH and VL domains connected with a flexible linker) at the C-terminus targeting the H antigen on the surface of red blood cells (RBCs). Patient serum/plasma containing antibodies targeting the SARS-CoV-2 RBD are mixed with RBCs and RBD-2E8 fusion protein and allowed to cross-link multiple RBCs in mass, eventually leading to visible agglutination seen with the naked eye. VH=variable heavy; VL=variable light. As a proof of concept, the RBD of the SARS-CoV-2 spike protein, corresponding to amino acids 330-524 of the spike protein (D. Wrapp, et al., Science. 367 (2020) 1260-1263), was chosen for its small size and stable folding, as well as the fact that the RBD is the target of the majority of neutralizing antibodies against coronaviruses (W. Tai, et al., Cellular and Molecular Immunology. 7 (2020) 226-8). Any positive test for antibodies binding to RBD would be highly suggestive of the presence of neutralizing antibodies that would be protective of reinfection (J. Sui, et al., Journal of Virology. 88 (2014) 13769-13780). Moreover, the RBD antigen has demonstrated to have the highest specificity toward SARS-CoV-2 to distinguish it from other coronaviruses, and 98% of patients develop RBD antibodies by day 9 of symptoms (L. Premkumar, et al., Sci Immunol. 5 (2020) eabc8413).

Using SARS-CoV-2 RBD as a target, two different fusion protein designs were prepared. The first one had RBD at the N-terminus, a short linker, and a C-terminal scFv against the H antigen, a carbohydrate antigen located within the ABO polysaccharides (E. A. Scharberg, et al., Immunohematology. 32 (2016) 112-118). The H antigen is ubiquitous in RBCs in the human population, except among Bombay individuals, who are exceptionally rare (M. Shrivastava, et al., Asian J Transfus Sci. 9 (2015) 74-77). A previous study indicated that the scFv 2E8 could successfully bind to RBCs and be used to display HIV gp41 peptides to detect HIV antibodies in a similar RBC agglutination assay (C. Shao and J. Zhang, Cellular and Molecular Immunology. 5 (2008) 299-306). The conditioned medium containing RBD-2E8 fusion protein was harvested from 293T cell culture after 72 hours of transfection of expression plasmids. The RBD-2E8 fusion protein was purified with a nickel column via His-tag affinity, and the RBD-2E8 protein was run on a protein gel to confirm the proper size (FIG. 2A).

The second fusion protein had a scFv, B6, on the N-terminus against an uncharacterized, ubiquitous RBC antigen (A. Gupta, et al., MAbs. 1 (2009) 268-280). B6 has been also used in fusion protein constructs to detect HIV antibodies in hemagglutination assays (A. Gupta, et al., J. Immunol. Methods. 256 (2001) 121-140). A short CH1 domain linker connects to RBD at the C-terminus, which improved agglutination previously by displaying the viral antigens further away from the RBC surface (G. Coia, et al., J. Immunol. Methods. 192 (1996) 13-23). This fusion protein, B6-CH1-RBD, was prepared in 293T cells according to the same protocol for RBD-2E8. B6-CH1-RBD was also prepared in E. coli as in prior studies for this class of diagnostic (A. Gupta and V. K. Chaudhary, J. Clin. Microbiol. 41 (2003) 2814-2821; and I. Habib, et al., Anal Biochem. 438 (2013) 82-89) with the thought this could be more scalable for manufacturing the reagents. Following nickel column purification via His-tag affinity, the proper size of B6-CH1-RBD bacterial protein on a protein gel was confirmed (FIG. 2B).

Testing red blood cell agglutination in a rapid assay to detect SARS-CoV-2 antibodies. First SARS-CoV-2 antibodies were tested in a rapid, 5-minute hemagglutination assay. The mammalian RBD-2E8 and bacterial B6-CH1-RBD fusion proteins were mixed with RBCs in the presence of COVID-19 patient serum in order to detect for agglutination. The assay was carried out in a small total volume, 40 μL, in a U bottom 96-well plate. The RBC solution (24% red blood cells) is at a dilution commonly used in manual tube testing for ABO typing in blood banks. This assay was validated via ABO antibody-mediated hemagglutination, finding visible, though subtle, agglutination already at 5-minutes (FIG. 3A).

Given that the amount of RBD-2E8 and B6-CH1-RBD fusion proteins necessary to cross-link antibodies and RBCs and trigger agglutination is unknown, a dilution series was performed, starting with a high concentration of the RBD-2E8 and B6-CH1-RBD stock solution (˜100 μg/mL) through 5 successive 1:1 dilutions. A negative control contained RBCs and COVID-19 patient serum without fusion proteins. After 5-minutes of incubation, no agglutination was seen with the bacterial B6-CH1-RBD protein (FIG. 3B). By contrast, moderate but perceptible agglutination was observed in the three higher concentrations of mammalian RBD-2E8 protein, with no agglutination observed in the more dilute RBD-2E8 concentrations (FIG. 3C). Decreasing levels of agglutination were observed along with a decreased concentration of RBD-2E8 (100%>50%>25%). Incubation of RBD-2E8 and RBCs without patient serum did not yield any agglutination (FIGS. 3B-C). This indicates the presence of antibodies that bind to RBD in the patient's serum, with agglutination activity reflective of RBD-2E8 concentration.

An extended assay yields more robust agglutination to detect SARS-CoV-2 antibodies. Because the 5-minute assay results were relatively weak making interpretation difficult, the assay was extended to one hour according to an agglutination protocol (A. Townsend, et al., A haemagglutination test for rapid detection of antibodies to SARS-CoV-2, medRxiv. (2020) 2020.10.02.20205831). The longer incubation would allow the RBCs to effectively come in contact with each other by gravity at the bottom of the well. The plate would then be tilted to observe if the red blood cells held together or fell down by gravity.

As before, this assay was validated via ABO antibody-mediated hemagglutination (FIG. 4A). Control antibodies of ACE2-Fc and CR3022 were tested to mediate agglutination, both of which are known to bind to SARS-CoV-2 RBD at two different, non-overlapping epitopes (J. Huo, et al., Nat Struct Mol Biol. 27 (2020) 846-854). For this assay, B6-CH1-RBD was produced by mammalian transfection, given the failure to mediate visible agglutination in the five-minute assay. Testing mammalian RBD-2E8 and B6-CH1-RBD, it was found that both antibodies could efficiently mediate agglutination of red blood cells that was visually clear from the PBS control, confirming RBD binding (FIG. 4B). Separately, the bacterial protein for this one-hour assay was tested with ACE2-Fc and CR3022, but again found no agglutination when different concentrations of B6-CH1-RBD were used. More specifically, bacterial B6-CH1-RBD protein failed to mediate agglutination of red blood cells. Different protein amounts of bacterial B6-CH1-RBD (100 ng, 250 ng, 500 ng, 750 ng, 1000 ng) were added into each well and mixed with ACE2-Fc (500 ng) and CR3022 (30 ng) diluted into 50 μL PBS and added to 50 μL red blood cells (2-4%). After one hour incubation and tilt test, no agglutination was observed at one hour.

Next, the analytical sensitivity of the assay was tested. Serial dilutions of CR3022 antibody were made and it was found that a level of 12.5 ng of antibody in the well could still trigger agglutination in the assay (FIG. 4C). Recovered COVID-19 patient serum was next tested. It was found that mammalian RBD-2E8 and B6-CH1-RBD fusion proteins could yield efficient agglutination reactions after one hour, which significantly more visually clear than the 5-minute incubation time (FIG. 4D). No agglutination was seen without fusion protein (FIG. 4D). Purified protein could yield agglutination compared to supernatant from transfected 293T cells, emphasizing the increased concentration of protein from nickel column purification. More specifically, the His-tagged purified protein versus unpurified supernatant yielded agglutination in the presence of COVID-19 patient serum. Twenty-five μL of RBD-2E8 and B6-CH1-RBD purified fusion protein or 25 μL of supernatant from the transfection of plasmids encoding these genes were tested in an agglutination reaction. As a control, 25 μL of PBS was added. Fifty μL of RBC's were added with 50 μL of COVID-19 patient serum. The His-tagged purified protein mediated agglutination as tested in a tilt test after one hour of incubation.

Discussion. The results described herein demonstrated the utility of a rapid, point-of-care RBC agglutination test for SARS-CoV-2 antibodies. The SARS-CoV-2 RBD was chosen as the target antigen for detection, since antibodies binding to the SARS-CoV-2 RBD have not exhibited cross-reactivity with other coronaviruses (L. Premkumar, et al., Sci Immunol. 5 (2020) eabc8413). RBD antibodies were maintained with little decrease through at least 75 days of follow-up, correlating with neutralizing antibody titers (A. S. Iyer, et al., Sci Immunol. 5 (2020) eabe0367). Based on available data then, an RBD fusion protein reagent could be used to reliably detect patients who have been infected and likely protected by neutralizing antibodies from infection.

These results demonstrated that COVID-19 patient serum could agglutinate RBCs in the presence of mammalian-derived RBD-2E8 within 5-minutes of incubation, although longer incubation for one-hour was required for strong agglutination and clear visualization. It was observed that the mammalian-derived RBD-2E8 fusion protein was more effective than the bacterial-derived B6-CH1-RBD fusion protein, which did not have any significant agglutination reaction in the 5-minute or 1-hour assay. A previous study on SARS RBD documented that mammalian-expressed RBD was significantly more reactive on ELISA to antisera from vaccinated mice than E. coli RBD protein (L. Du, et al., Virology. 393 (2009) 144-150). It is likely that the native glycosylation and RBD folding in mammalian cells is useful for efficient antibody recognition. This result differs from previous studies to detect HIV antibodies, where p24 fusion protein could be expressed in E. coli and efficiently cause hemagglutination reactions (A. Gupta and V. K. Chaudhary, Protein Expr. Purif. 26 (2002) 162-170; and I. Habib, et al., Anal Biochem. 438 (2013) 82-89).

The method does not distinguish between IgG, IgA, or IgM against SARS-CoV-2, which may be desired in certain clinical scenarios. IgG subclasses can similarly not be distinguished. While the assay is simple and can be read with the naked eye, there is more subjectivity to it compared to lateral flow assays or chemiluminescent ELISA's. A negative control test without fusion protein will be important to include during clinical implementation, given that rare patients may have false positives from agglutination-inducing IgM autoantibodies (J. Yudin and N. M. Heddle, Lab Med. 45 (2014) 193-206). Ongoing studies will confirm the sensitivity and specificity of the assay with more COVID-19 patient samples. Similar prior hemagglutination assays for HIV antibodies demonstrated 100% sensitivity (n=94) and 99.5% specificity (n=596) (K. M. Wilson, et al., J. Immunol. Methods. 138 (1991) 111-119).

The hemagglutination assay described herein can use a drop of whole blood from a patient finger-stick, wherein RBD-2E8 or B6-CH1-RBD fusion protein alone could be added for the assay. The cost of the assay is financially feasible in low-resource health care settings. While the assay was carried out in 96-well plates similar to previous studies (C. Shao and J. Zhang, Cellular and Molecular Immunology. 5 (2008) 299-306), the format is transferrable to slide agglutination test (A. Gupta and V. K. Chaudhary, J. Clin. Microbiol. 41 (2003) 2814-2821), which is often used for ABO testing in low-resource settings. Point-of-care applications are emphasized in this study, but fusion protein reagents could potentially be employed in other hemagglutination assays used in clinical labs, such as tube testing and gel card testing, as well as on automated solid-phase assay machines. Beyond diagnostic use of COVID-19 patients, the ability to rapidly screen donors for RBD antibodies in the plasma of COVID-19 recovered patients could facilitate the continued deployment and scaling of convalescent plasma as a therapy against COVID-19 (R. L. Kruse, Therapeutic strategies in an outbreak scenario to treat the novel coronavirus originating in Wuhan, China, F1000Res. 9 (2020) 72; and E. M. Bloch, S et al., J. Clin. Invest. (2020)). Rapid testing would also help validate RBD antibody production induced by SARS-CoV-2 vaccines in trials, and screen for patients in need of the vaccine. In conclusion, the RBC agglutination assay with cross-linked viral antigen-antibody fusion is a simple, cheap, and scalable way to dramatically increase the capability of detecting antibodies against SARS-CoV-2. It has utility, particularly in low-resource settings, in the efforts to combat the COVID-19 pandemic.

### Example 4: A Hemagglutination-Based, Semi-Quantitative Method for Point-of-Care SARS-CoV-2 Antibody Detection

Serologic, point-of-care tests to detect antibodies against SARS-CoV-2 are an important tool in the COVID-19 pandemic. The majority of current point-of-care antibody tests developed for SARS-CoV-2 rely on lateral flow assays, but these do not offer quantitative information. To address this, a method of COVID-19 antibody testing was developed employing hemagglutination tested on a dry card. A fusion protein linking red blood cells (RBCs) to the receptor-binding domain (RBD) of SARS-CoV-2 spike protein was placed on the card. Two-hundred COVID-19 patient samples and 200 control serum samples were reconstituted with 0-negative RBCs to form whole blood and added to the dried protein, followed by a stirring step, 3-minute incubation, and a second stirring step. Among recovered COVID-19 patients, the hemagglutination test detected antibodies in 87% of patients. The Euroimmun IgG ELISA test detected antibodies in 86.5% of the same samples, while the RBD-based CoronaChek lateral flow assay was 84.5% sensitive on the same samples. Testing pre-pandemic samples, the hemagglutination test had a specificity of 95.5%, compared to 97.3% and 98.9% for the ELISA and CoronaChek, respectively. A distribution of agglutination strengths was observed in convalescent COVID-19 samples, which correlated with neutralizing antibody titers. Strong agglutinations were observed within 1 minute of testing, corresponding with specimens that had higher neutralizing antibody titers. Shorter assay time could also increase specificity to 98.5%. In conclusion, a rapid, point-of-care RBC agglutination test for the detection of SARS-CoV-2 antibodies was developed that can yield semi-quantitative information on neutralizing antibody titer in patients. The five-minute test can be used in evaluating vaccinated populations as a method of proving and monitoring immune responses.

The COVID-19 pandemic has brought enumerable healthcare morbidity and mortality on to the world, as well as economic and social disruption. The size and spread of SARS-CoV-2 infection has brought numerous challenges, including many diagnostic challenges. This includes the diagnosis of the initial infection itself, but also monitoring individuals for immune responses against SARS-CoV-2. This has led to a rush of development of SARS-CoV-2 serology assays onto the market for monitoring antibody development.

The presence of SARS-CoV-2 antibodies has been associated with prevention of re-infection in large systemic studies, wherein a cohort of millions of individuals had a 90% reduction in a subsequent positive NAAT test if they had antibodies (Harvey R A, et al. JAMA Intern Med 2021). In addition, SARS-CoV-2 vaccine trial efficacy has been associated with the titer of antibodies that are induced by the vaccines, which led to differing levels of protection to symptomatic infection (Earle K A, et al. Evidence for antibody as a protective correlate for COVID-19 vaccines. medRxiv 2021;:2021.03.17.20200246). The presence of antibodies has also been important for determining the efficacy of monoclonal antibody therapies against COVID-19 (Weinreich D M, et al. N Engl J Med 2021; 384(3):238-51). Patients negative for antibodies at diagnosis were found to have a significant decrease in hospitalization, while antibody-positive patients did not gain significant improvement from monoclonal antibody therapy (Weinreich D M, et al. N Engl J Med 2021; 384(3):238-51).

More recent studies have shown that individuals previously recovered from COVID-19 just need a single dose of vaccine to achieve similar benefit to naïve individuals receiving two doses (Wu F, et al. JAMA Intern Med 2020; 180(10):1356-62). Implementing this policy could reduce the total amount of vaccine doses, but would require a rapid method of screening individuals for antibodies to confirm previous infection. Rapid testing options for SARS-CoV-2 antibodies have relied on lateral flow assays, but the quality of later flow tests approved under emergency use authorization has been variable. Furthermore, lateral flow tests do not offer any quantitative feedback on antibody levels, which is important given the wide range of antibody responses, particularly among mild cases of SARS-CoV-2 infection (Wu F, et al. JAMA Intern Med 2020; 180(10):1356-62).

Methods. Specimens. The characteristics of the specimens were previously described (Conklin S E, et al. J Clin Microbiol 2021; 59(2); and Patel E U, et al. J Clin Microbiol 2021; 59(2)). The samples were deidentified prior to testing. The current research includes an analysis of stored samples and data from those studies. No additional samples were collected for the current study.

Briefly, the convalescent SARS-CoV-2 samples were based on patients who were confirmed RT-PCR positive and at least asymptomatic for 28 days (average 45±7.5 days). The pre-pandemic samples were collected from a prior study of patients presenting to the Johns Hopkins Hospital Emergency Department with symptoms of an acute respiratory tract infection between January 2016 and June of 2019.

Serological Assay testing using commercial assays. Lateral flow assays (see Table 4 and Table 4 for complete list) and the Euroimmun IgG Spike ELISA were conducted under manufacturer's protocols and the data previously acquired during other studies on the same specimens (Conklin S E, et al. J Clin Microbiol 2021; 59(2)). Neutralizing antibody assays were also performed previously (Patel E U, et al. J Clin Microbiol 2021; 59(2)).

TABLE 4
Hemagglutination-based assay performance. Sensitivity and specificity
are presented for the hemagglutination test using 200 samples of
PCR-confirmed COVID-19 patients and 200 pre-pandemic samples of
patients with acute respiratory symptoms. Results of a regulatory-
approved Euroimmun Spike IgG ELISA test and RBD-based CoronaChek
lateral flow test on the same samples are also presented for comparison.
Specificity results are presented for an equivalent bank of pre-
pandemic samples, although not all samples overlap between the three
groups. Borderline samples on ELISA were called positive, and faint
samples on lateral flow assay were called positive.

| Specimens | Sensitivity | Analysis |
|---|---|---|
| Hemagglutination Test | 87.0% | 174/200 samples |
| Euroimmun Spike IgG ELISA | 86.50% | 173/200 samples |
| CoronaCheck Lateral Flow Assay | 84.50% | 169/200 samples |
| Specimens | Specificity | Analysis |
| Hemagglutination Test | 95.5% | 191/200 samples |
| Euroimmun Spike IgG ELISA | 97.29% | 502/516 samples |
| CoronaCheck Lateral Flow Assay | 98.97% | 574/580 samples |

TABLE 5
Comparison of the hemagglutination test to lateral flow tests.
Lateral flow tests were compared to the hemagglutination
based test on the same samples from recovered patients,
representing a subset of the 200 PCR-confirmed COVID-19
patients presented previously. The number of samples available
for comparison are listed, as well as the calculated sensitivity
and number of positive samples for both the lateral flow
assay and hemagglutination test presented.

| | | | Hemagglutination |
|---|---|---|---|
| | Samples | Sensitivity | Test Sensitivity |
| Lateral Flow Assay | Tested | (%, # positive) | (%, # positive) |
| Biomedomics | 58 | 86.21% (50) | 94.83% (55) |
| Innovita | 20 | 95% (19) | 95% (19) |
| Smart Screen | 25 | 60.00% (15) | 92.00% (23) |
| One Milo | 21 | 66.67% (14) | 90.48% (19) |
| Premier BioTech | 21 | 95.24% (20) | 90.48% (19) |
| Ready Results | 25 | 80.00% (20) | 92.00% (23) |
| Sensing Self | 25 | 84.00% (21) | 92.00% (23) |
| AYTU | 21 | 71.43% (15) | 90.48% (19) |
| ZEUS | 21 | 52.38% (11) | 90.48% (19) |
| Wondfo IgM & IgG | 21 | 42.86% (9) | 90.48% (19) |
| Covisure | 21 | 57.14% (12) | 90.48% (19) |
| All Test | 21 | 90.48% (19) | 90.48% (19) |
| Clarity | 21 | 76.19% (16) | 90.48% (19) |
| Nirmidas | 21 | 95.24% (20) | 90.48% (19) |
| SafeCare | 21 | 90.48% (19) | 90.48% (19) |
| DNA Link | 21 | 95.24% (20) | 90.48% (19) |
| TBG | 21 | 95.24% (20) | 90.48% (19) |
| Wantai Total Ab | 46 | 69.57% (32) | 93.48% (43) |

Hemagglutination Dry Card production. Eldon Biologicals currently sells cards with dried antibodies (EldonCards) to detect ABO and Rh for blood typing. These cards were repurposed for COVID-19 antibody detection and formulated by Eldon Biologicals instead with the IH4-RBD (SEQ ID NO: 42) fusion protein (Townsend A, et al. Nat Commun 2021; 12(1):1951-12). The IH4-RBD fusion protein was obtained from Absolute Antibody (Oxford, United Kingdom) (Townsend A, et al. Nat Commun 2021; 12(1):1951-12). 533.2 ng of IH4-RBD protein was dissolved in a proprietary buffer and placed onto the card. The cards were then heated to leave a dried protein mixture on the card, which is stable at room temperature and can be packaged and shipped.

Sample preparation. Type O, Rh-negative packed red blood cells (pRBC's) were obtained from Tennessee Blood Services and provided by Biochemed Services (Winchester, Va.). pRBC's were promptly stored at 4° C. by receipt and used entirely within 28 days for the testing. The red blood cells were washed with PBS to remove any residual plasma. Washed pRBC's were combined with frozen serum to reconstitute “whole blood” with ˜40% hematocrit after combining pRBC's and frozen serum.

Testing protocol. For each test, 20 μL of tap water was placed onto the dried protein spot within the test circles on the card to dissolve the protein. Twenty μL of reconstituted whole blood was then added to the spot. The fluid of water and blood was mixed with a plastic Eldon Stick, spreading the liquid completely within the circle to make sure that the dissolved protein was mixed well with blood on the card. The card was tiled for 10 seconds in each 90 degree direction (4 times in total) and allowed to incubate on a flat surface for 3 minutes. The card was then tilted again as before for 4 times in each 90 degree direction.

Tests were interpreted according to similar protocols established for scoring hemagglutination in EldonCard blood typing assays. The tests were assigned scores of 4, 3.5, 3, 2.5, 2, 1, and 0. The scores of 1 and 0 were assigned as negative results, where a score of 2 or higher was a positive test result. Tests were interpreted both during tilting of the card, as well as interpretation on a flat horizontal surface, since weak agglutinations could be appreciated in certain cases more easily with the liquid droplet on the side.

Statistics. Statistical analyses were performed using GraphPad Prism 9. Statistical comparisons made between groups comparing neutralizing antibody titers were made using non-parametric Mann-Whitney tests. Statistical significance was set at P<0.05.

Results. Validation of hemagglutination, point of care test. Studies have demonstrated the capability of using a fusion protein of an antibody against a red blood cell antigen connected to the receptor binding domain (RBD) of SARS-CoV-2 to detect antibodies against RBD in patient serum (Kruse R L, et al. Biochemical and Biophysical Research Communications 2021; 553:165-71; and Townsend A, et al. Nat Commun 2021; 12(1):1951-12). The RBD is a small subunit of spike protein with a sequence that is highly differentiated from other seasonal coronaviruses and thus should have less cross-reactivity (Premkumar L, et al. Sci Immunol 2020; 5(48)). The goal was to adapt the methods disclosed herein, which required pipettes, 96-well plates, and an hour long of incubation, into one that could be distributed as a consumer, point-of-care test.

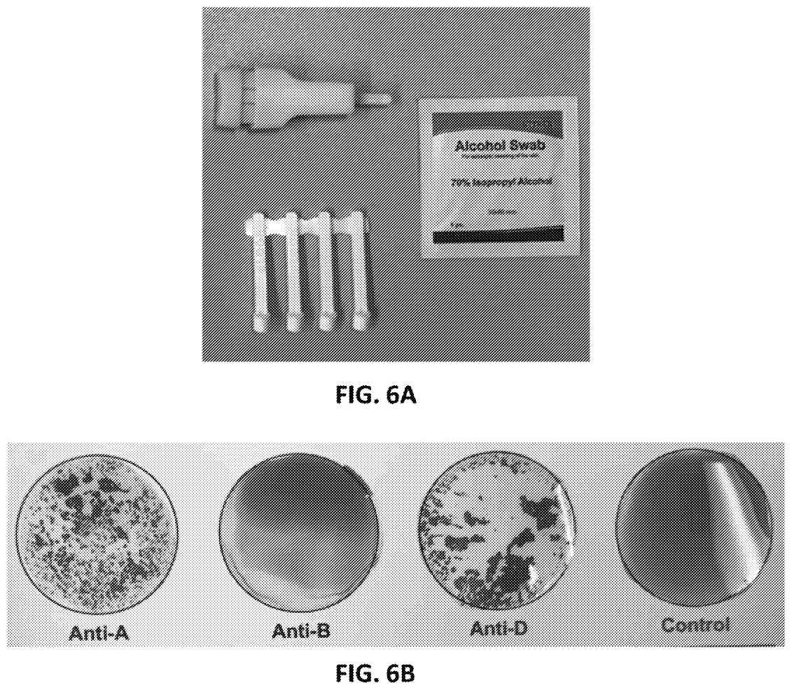

Toward this goal, the dry hemagglutination cards, which are currently used in countries across the world in a room temperature stable kit for rapid, point-of-care testing for ABO and Rh-blood types were adapted. Examples of uses include blood-typing mothers at the time of birth and blood-typing soldiers in need of emergent transfusion in the battlefield. For example, the hemagglutination card kits comes with components of a lancet to elicit blood, a dropper to add water to the platform, as well as stirring sticks to develop the assay. For example, FIG. 6A shows an Eldon Card that itself can have antibodies against ABO and Rh blood groups dried onto spots on the card. Each test circle has dried antibodies to the target RBC antigen to trigger hemagglutination and typing determination (FIG. 6B).

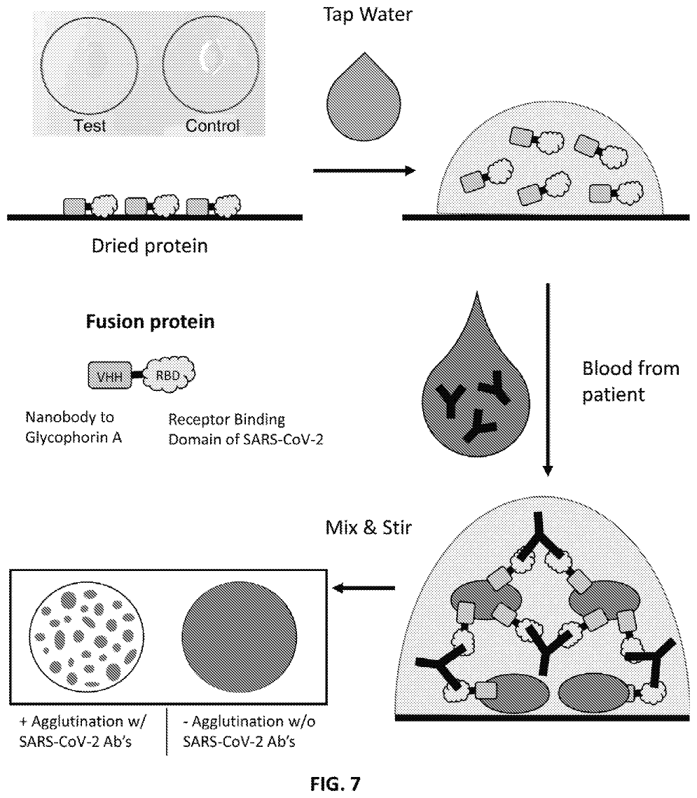

This platform was used to develop a rapid antibody test for SARS-CoV-2 that could be used in similar low-resource settings with the same cost-effective and scalable manufacturing platform. As outlined in FIG. 7, a fusion protein, for example, IH4-RBD (Townsend A, et al. Nat Commun 2021; 12(1):1951-12), and dried it onto a hemagglutination card to formulate the test. Addition of water solubilizes the fusion protein, and addition of blood containing COVID-19 antibodies facilitates cross-linking of RBC's, which after stirring, can be observed macroscopically (FIG. 7).

A bank of frozen serum samples was obtained representing COVID-19 convalescent patients, along with pre-pandemic samples of patients with respiratory illness symptoms. Serum was reconstituted with O-negative blood to a hematocrit ˜40% and placed onto the card. A protocol was developed for testing. The protocol included a stirring step for 1 minute, followed by a 3 minute incubation, and then a 1 minute stirring step, followed by test visualization. For example, a vaccinated individual can use the protocol described to detect antibodies on the EldonCard. The assay is stopped after the first round of card tilting when a strong agglutination is observed. Weaker agglutinations would continue with three minutes of incubation time, followed by a second round of card tilting.

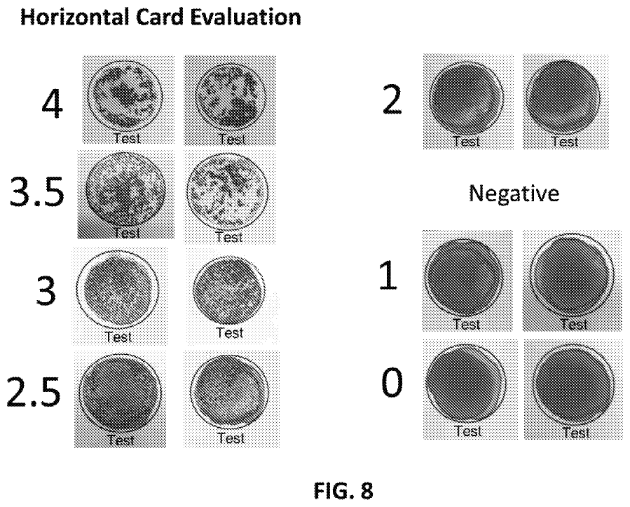

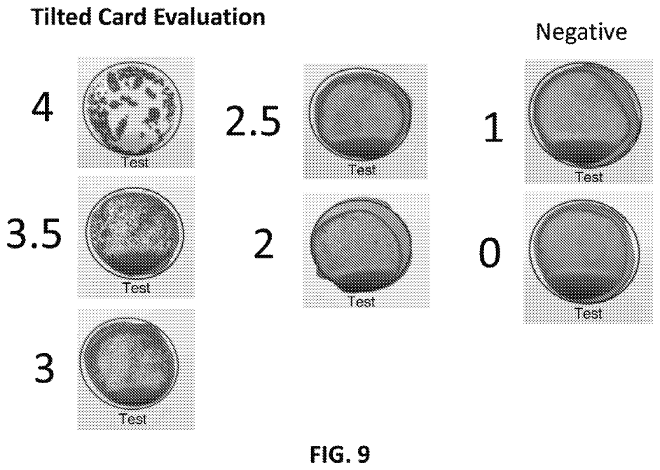

Hemagglutination test performance against clinical samples. Testing on clinical samples was performed from patients with PCR-confirmed infection. The agglutinations observed on the card across 200 samples tested and scored. As shown in FIG. 8, the highest agglutination was scored at 4, wherein large clumps of red cells are seen with few residual free cells, to 0 wherein no reaction is observed. The agglutination scores of 0 and 1 were termed to be negative, while any score at 2 or above was positive. Scoring is presented as the cards resting on a horizontal surface in FIG. 8, as well as slanted after final mixing (FIG. 9). In the latter scenario, it can often be easier to see small agglutinations without the large liquid droplet of un-agglutinated red cells obscuring the view, as well as the kinetic movement of these agglutinations across the test circle. In an agglutination score 1 field, there can be some small number of agglutinations observed, but these are usually very few and often fixed to card, and do not move like most agglutinations in a 2 score.

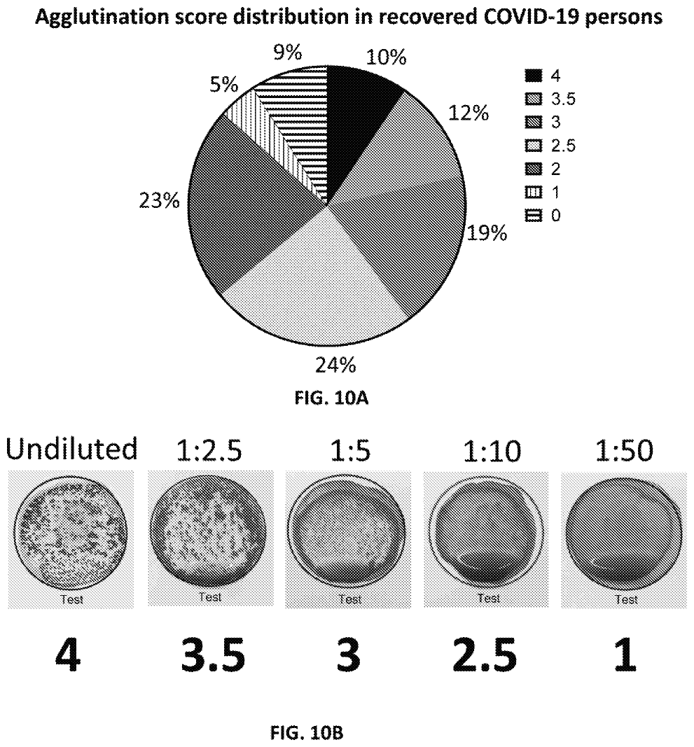

Next, these differences in agglutination were evaluated to confirm whether they are related to the antibody concentration in the serum, which would influence the amount of red blood cell cross-linking observed. Using a patient sample that scored a 4 agglutination and a serial dilution was performed in order to assess whether agglutination decreased. A decline in agglutination correlated progressively with more dilute samples (FIG. 10B), with a 1:10 dilution scoring a clear, but weak reaction, while a 1:50 dilution did not show a clear reaction after 5 minutes. Thus, the agglutination results correlate with antibody concentration.

Across the 200 recovered COVID-19 patients, a range of different agglutination scores were observed (FIG. 10A). Interestingly, relatively few patients achieved the highest levels of agglutination 4 and 3.5, while 47% patients had borderline studies (2-2.5). The relationship between agglutination score and neutralizing antibodies against SARS-CoV-2 was next determined, showing general correlation with increasing agglutination score and higher neutralizing antibody titers against the virus (FIG. 10B). Notably, agglutination scores of 1 and 2 had no difference in neutralizing titer, while strong agglutination scores 3 or higher were clearly defined by higher neutralizing antibody levels.

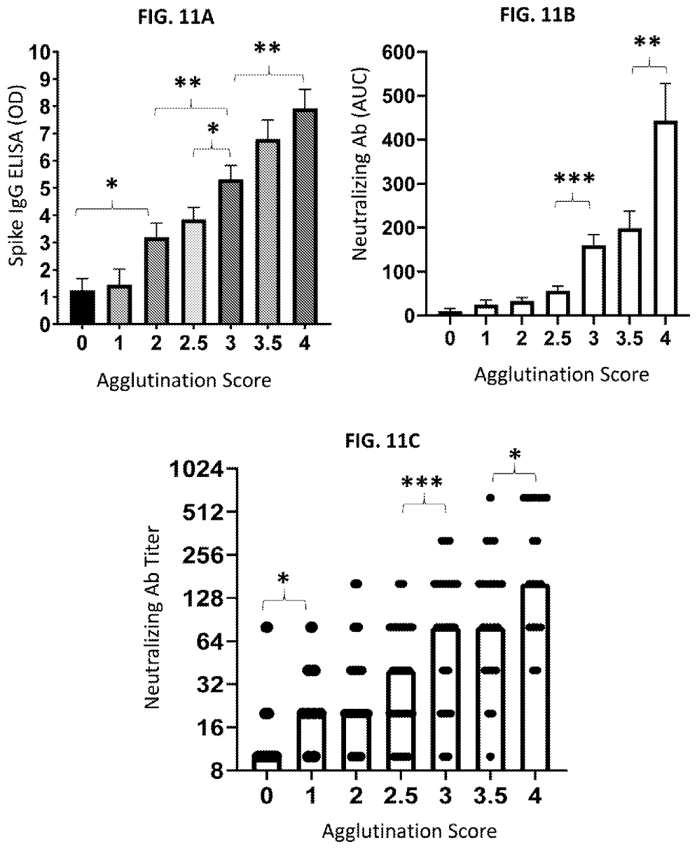

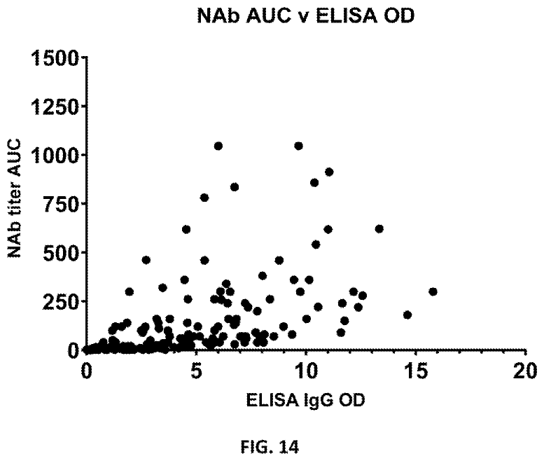

The relationship between agglutination score and traditional ELISA and neutralizing antibody assays against SARS-CoV-2 was next determined. A correlation between the optical density of the Spike IgG ELISA assay and agglutination score was observed, despite the hemagglutination assay containing RBD, which is a small portion of the Spike protein (FIG. 11A). The RBD is a major target of neutralizing antibodies, so the agglutination score was examined versus neutralizing antibody levels. A general correlation was observed between increasing agglutination score and higher neutralizing antibody levels for both the AUC (FIG. 11B) and endpoint dilution titer (FIG. 11C) against the virus. Notably, agglutination scores of 1 and 2 had no difference in neutralizing antibodies, while strong agglutination scores 3 or higher were clearly defined by higher neutralizing antibody levels. By contrast to agglutination score, the Spike ELISA OD and neutralizing antibody AUC had a weaker correlation to each other (FIG. 14).

The accuracy of the tests was next calculated across the 200 samples of recovered COVID-19 patients, and 200 pre-pandemic samples from patients with respiratory symptoms. The sensitivity for detecting antibodies was 87.0%. By comparison, the FDA-approved Euroimmun Spike IgG ELISA test showed a sensitivity of 86.5%, while a high-performing RBD-based lateral flow assay, CoronaChek, 84.50% on the same 200 samples (Table 4). Specificity for the hemagglutination test was calculated at 95.5%, which was lower than the Euroimmun ELISA (97.29%) and CoronaChek (98.97%), respectively. A cohort of additional lateral flow assays were also compared to the hemagglutination test on a smaller set of samples (Table 5), with the hemagglutination test performing similar or significantly better than 18 lateral flow assays utilized.

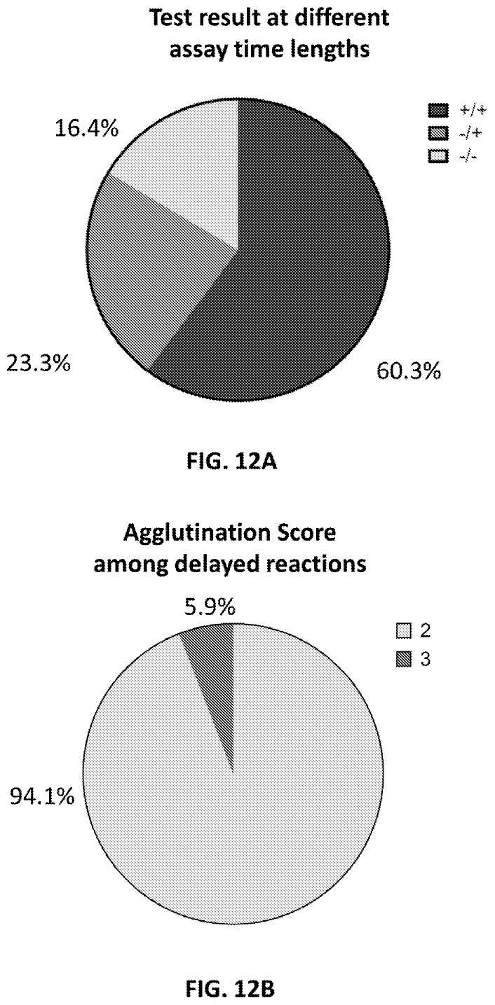

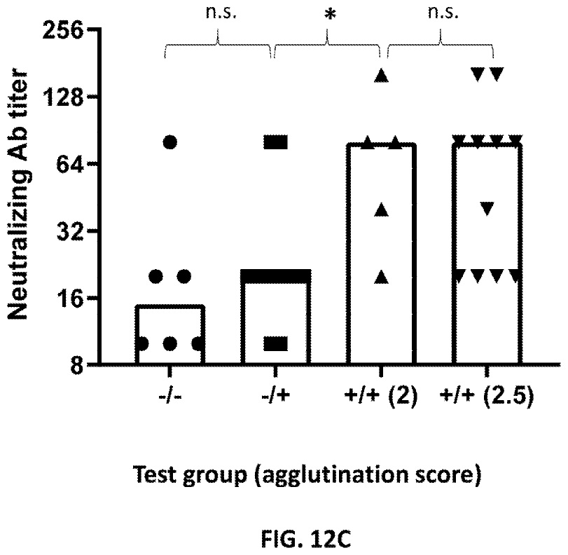

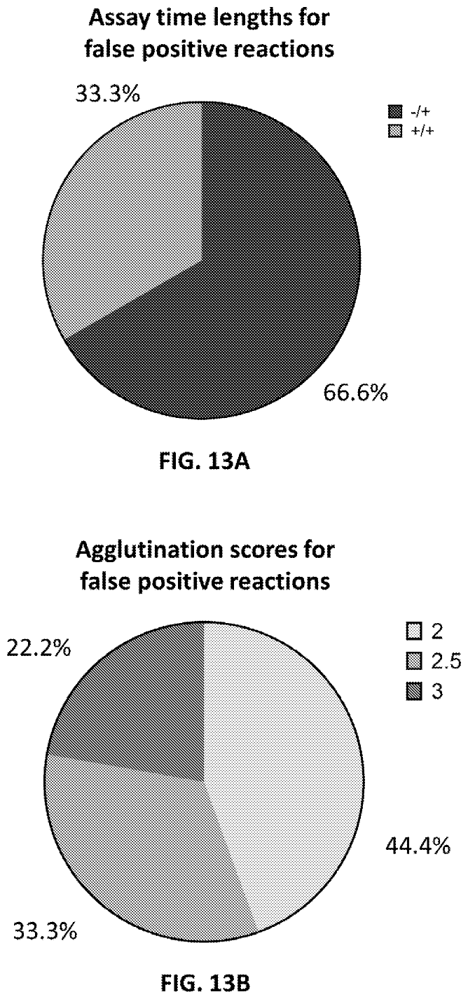

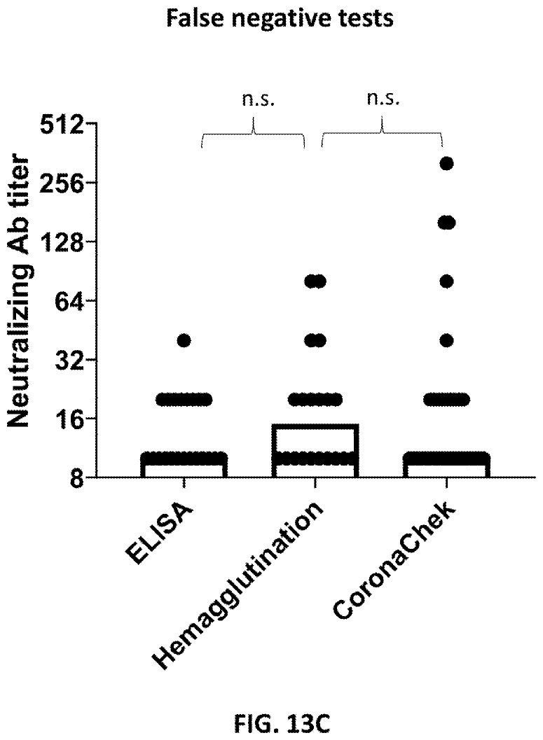

Analysis of false positive and negative samples. Next, it was determined how the assay time influenced the test performance observed, given that many strong agglutination reactions were observed after just the first series of card tilting and blood mixing. In a smaller cohort of 73 tests within the 200 samples, the test was scored after initial stirring as well as after the complete assay time. It was found that the sensitivity of the assay decreased to 60.3% if a one-minute of assay time was leveraged (FIG. 12A). The additional incubation time could pick up another (23.3%) of samples, although the agglutination scores of these tests were almost 2 (94.1%) (FIG. 12B). Interestingly, the neutralizing antibody titers of these weak reactions requiring additional incubation time were very low and were significantly lower than the neutralizing antibody titers for similarly weak reactions (2-2.5) that were initially visible after 1-minute (FIG. 12C). Together, this points to increased sensitivity, but lower functional utility in increasing assay time. The false positive samples in the pre-pandemic samples were next interrogated. Among the 9 false positives, 6 required the additional incubation time to become positive (FIG. 13A). The agglutination scores among the false positives were also weak, with 2 out of 9 registering a score of 3 (FIG. 13B). If the assays were limited to just interpretations after 1-minute, the specificity would increase to 98.5% (197/200). The false negatives among the ELISA, hemagglutination, and CoronaChek assays were next compared. While there were no statistical differences between tests, the lateral flow test had a trend toward higher levels of neutralizing antibodies completely missed (FIG. 13C). Interestingly, one sample measured 1:320 neutralizing antibody titer, a score of 4 by agglutination, but was negative on the CoronaChek test.

Discussion. In this study, a new SARS-CoV-2 antibody testing platform at the point of care was established that is distinguished from the commonly used lateral flow assays for detecting antibodies. It was found that this hemagglutination-based platform is faster than lateral flow assays (5 minutes vs 15 minutes) and ELISA assays by comparison, and can achieve similar sensitivities, when tested on the same samples. Moreover, the components to do the tests are simple.

The results demonstrate an 87.0% sensitivity among COVID-19 recovered patients, which compared well to a leading ELISA (86.5%) and lateral flow assay (84.5%) already used in patients today. This sensitivity finding was similar to the prior study using the IH4-RBD fusion in a 96-well plate with one-hour incubation, which found a 90% sensitivity (Townsend A, et al. Nat Commun 2021; 12(1):1951-12). The slight differences in sensitivity between the two assays may result from the longer incubation time and gravity in a 96-well, U-bottom plate facilitating RBC aggregation. While many ELISA and lateral flow assays use the whole spike protein, the receptor binding domain (RBD) was used for this test, since it is the main target of neutralizing antibodies, which should provide protective immunity. Technically, the RBD is also much smaller and can be easily manufactured into a fusion protein.

RBD has been employed as the target antigen in ELISA (Amanat F, et al. Nature Medicine 2020; 26(7):1033-6) and lateral flow tests (Conklin S E, et al. J Clin Microbiol 2021; 59(2)), respectively. Some results from RBD-based ELISA tests have found extremely high sensitivity (98%) and specificity (100%) (Premkumar L, et al. Sci Immunol 2020; 5(48):eabc8413)) and IgG sensitivity of 96% and specificity of 99.3% after 10 days of symptom onset (Peterhoff D, et al. Infection 2021; 49(1):75-82). While these are much higher than the results described herein, another RBD-based ELISA assay had results more similar to this study, with an 88% sensitivity for IgG against RBD, and 98% specificity in a population in Israel (Indenbaum V, et al. PLoS ONE 2020; 15(11):e0241164). As observed in the sub-group analysis comparing to different lateral flow assays, the testing performance is reported is strongly dictated by antibody levels in the specimens, which is in turn dictated by the patient population, with severe disease patients having higher antibody levels than mild cases (Lau E H Y, et al. Nat Commun 2021; 12(1):63-7). This limits comparisons between different papers for reported sensitivity and specificity papers and emphasizes the need for comparison of tests on the same set of samples.

When analyzing sensitivity, it has been observed that not every patient recovered from COVID-19 produces antibodies at high levels or in some cases at all, particularly among non-hospitalized cases with mild to no symptom (Premkumar L, et al. Sci Immunol 2020; 5(48)). This has likely led to low sensitivities when samples are tested against patients from the full spectrum of disease, which was reflected in the test performance of the hemagglutination tests disclosed herein, as well as ELISA and lateral flow assay comparators. Importantly, while patients may develop antibodies to the spike protein, some rare patients do not develop appreciable responses to RBD (Premkumar L, et al. Sci Immunol 2020; 5(48):eabc8413). While this would cause a false negative in the instant test, the functional consequences for lack of immunity against RBD may still have clinical utility. Moreover, the S2 domain is well-known to be more cross-reactive for antibodies with other seasonal coronaviruses (Khan S, et al. Analysis of Serologic Cross-Reactivity Between Common Human Coronaviruses and SARS-CoV-2 Using Coronavirus Antigen Microarray. bioRxiv 2020; 2020.03.24.006544).

The specificity in the disclosed assay (95.5%) was lower than the 99% reported using the same fusion protein previously, and also lower than the aforementioned RBD-based ELISA tests (specificity of 100% (Premkumar L, et al. Sci Immunol 2020; 5(48):eabc8413) and 99.3% (Peterhoff D, et al. Infection 2021; 49(1):75-82)). The reason for lower specificity is uncertain but is likely multi-factorial. The manufacturing of a dried protein on the card may yield fusion protein clumping not seen in protein in solution, while the use of excipients in the EldonCard to facilitate faster, stronger agglutinations that may also precipitate a higher degree of false positives among “false agglutinations.” Another consideration is that the prior study (Townsend A, et al. Nat Commun 2021; 12(1):1951-12) tested healthy donors as a control, while the negative control samples in the assay used in this Example were patients with acute respiratory illness, including a subset with active seasonal coronavirus infection. While the sequence identity is ˜20% shared between the viruses (Li D, and Li J. Immunologic testing for SARS-CoV-2 infection from the antigen perspective. J Clin Microbiol. 2021 Apr. 20; 59(5):e02160-20.) it's possible that even weakly cross-reactive antibodies could achieve binding at high concentrations. Cross-reactivity has been suggested as a reason for significantly worse specificity of SARS-CoV-2 antibody ELISA assays in African populations (90-94% against spike protein) (Emmerich P, et al. Trop Med Int Health. 2021 Mar. 5; 10.1111/tmi.13569). An important distinction is that ELISA cutoff values for optical density can be optimized for maximal specificity (Perera R A, et al. Serological assays for severe acute respiratory syndrome coronavirus 2 (SARS-CoV-2), March 2020. Euro Surveill 2020; 25(16)), whereas the hemagglutination test relies on visual interpretation with more limited nuance. Of note, specificity could be increased up to 98.5% if assay time was reduced to 1-minute, suggesting that these false positives could be eliminated with further modification of the assay.

Although the instant hemagglutination test on a dry card cannot be used to distinguish between IgG/IgM/IgA, an important advantage of this test is the semi-quantitative readout of antibody levels, which is different compared to the currently available point of care COVID-19 serology assays. A correlation between agglutination score and neutralizing antibody titer was observed, which has also been seen in RBD-based ELISA assays where a correlation with neutralizing antibody titer was found (Premkumar L, et al. Sci Immunol 2020; 5(48):eabc8413). The ability to interpret an agglutination pattern for semi-quantitative determination was previously used in the SimpliRED test, gauging D-dimer levels at the point of care (Neale D, et al. Emerg Med J 2004; 21(6):663-6)). Importantly, the correlation between neutralizing antibody levels and agglutination can also help refine use cases for the test in the future, such that scores lower than 3 are determined to lack substantial immunity. As mentioned above, specificity could also be improved, given that non-reacting tests at 1-minute do not have high levels of neutralizing antibodies anyways. Of note, preliminary testing of vaccinated individuals with the instant hemagglutination test has shown agglutinations of 3.5 or 4, matching the uniformly high levels observed in clinical trials. One study has explored the potential for interpreting strong and weak lines from lateral flow assays to correlate with antibody levels (Weidner L, et al. J Clin Virol 2020; 129:104540), but such strategy hasn't been translated into commercial instructions and is visually more subtle than the agglutination results presented in this study.

In summary, a new platform for SARS-CoV-2 antibody detection was developed that is faster than current point-of-care devices and offers semi-quantitative information. The simplicity and cheap cost of the platform could enable its widespread use in low resource settings and help contribute to the end of the current pandemic. Further, this test can be used to rapidly screen individuals before vaccination, in order to determine if they possess high or low antibody levels from a previous COVID-19 infection and could be useful in monitoring declining antibody levels in patients over time.

What is claimed is:

1. A recombinant polypeptide comprising:
a first domain, wherein the first domain comprises an epitope of a coronavirus;
a linker; and
a second domain, wherein the second domain is a moiety that is capable of specifically binding an antigen on the surface of a red blood cell.

2. The recombinant polypeptide of claim 1, wherein the epitope of a coronavirus is an epitope of a severe acute respiratory syndrome coronavirus 2 (SARS-CoV-2), a severe acute respiratory syndrome (SARS), middle east respiratory syndrome (MERS), a human coronavirus 229E, a human coronavirus NL63, Miniopterus bat coronavirus 1, a Miniopterus bat coronavirus HKU8, porcine epidemic diarrhea virus, a Rhinolophus bat coronavirus HKU2, a Scotophilus bat coronavirus 512, a bovine coronavirus, a human coronavirus OC43, a human coronavirus HKU1, murine coronavirus, a Pipistrellus bat coronavirus HKU5, a Rousettus bat coronavirus HKU9, a Tylonycteris bat coronavirus HKU4, a hedgehog coronavirus 1, an infectious bronchitis virus, a beluga whale coronavirus SW1, an infectious bronchitis virus, a Bulbul coronavirus HKU11, a pangolin coronavirus, a porcine coronavirus HKU15, a WIV1-CoV, a SHC014-CoV, a bat-SL-CoVZC45, a bat-SLCoVZXC21, a SARS-CoVGZ02, a BtKY72, a WIV16, Rs4231, a Rs7327, a Rs9401, a BtRs-BetaCoV/YN2018R, a BtRs-BetaCoV/YN2013, Anlong-112, a Rf2092, a BtRs-BetaCoV/YN2018C, a As6526, Rs4247, a BtRs-BetaCoV/GX2013, a Yunnan2011, a BtRl-BetaCoV/SC2018, a Shannxi2011, a BtRs-BetaCoV/HuB2013, a Bat_CoV_279/2005, a HKU3-12, a HKU3-3, a HKU3-7, a Longquan-140, or a RaTG13.

3. The recombinant polypeptide of claim 1 or 2, wherein the first domain comprises a sequence from a coronavirus spike (S) protein, envelope (E) protein, a membrane (M) protein, a nucleocapsid (N) protein, an antigenic fragment thereof, or a combination thereof.

4. The recombinant polypeptide of claim 3, wherein the first domain comprises a sequence from a coronavirus spike (S) protein, wherein the S protein sequence comprises a S1 domain, a S2 domain, the N-terminal domain, a receptor-binding domain or the entire S protein ectodomain.

5. The recombinant polypeptide of claim 4, wherein the first domain comprises a sequence from a coronavirus nucleocapsid (N) protein, wherein nucleocapsid protein has the dimerization domain removed or nucleocapsid polypeptide fragments do not include the dimerization domain.

6. The recombinant polypeptide of claim 1, wherein the first domain comprises one or more of SEQ ID NOs: 5-15 or an antigenic fragment thereof.

7. The recombinant polypeptide of claim 1, wherein the second domain is a single chain variable fragment (scFv), a Fab, a camelid antibody, a nanobody, a shark vNAR antibody, adnectins, anticalins, avimers, Fynomers, Kunitz domains, knottins, affibodies, β-hairpin mimetics, designed ankyrin repeat proteins, or a peptide capable of binding to an antigen on the surface of a red blood cells.

8. The recombinant polypeptide of claim 7, wherein the scFv is derived from an antibody capable of specifically binding to an antigen on the surface of a red blood cells.

9. The recombinant polypeptide of claim 7, wherein the scFv is 10F7, A41, B6, 2E8, 1C3, ABO.B1, ABO.HI1, Rh.D1, Rh.E1, Ery.X1, K.Kpb1, 4G11, or a single domain antibody IH4.

10. The recombinant polypeptide of claim 1, wherein the second domain is 10 to 100 amino acids long and is derived from an erythrocyte-binding sequence, wherein the erythrocyte-binding sequence is from a Plasmodium protein, wherein the Plasmodium protein is SERA, STEVOR, erythrocyte binding antigen-175, erythrocyte binding antigen-181, erythrocyte binding antigen-140, erythrocyte-binding ligand-1, or PfGARP.

11. The recombinant polypeptide of claim 1, wherein the second domain comprises one of SEQ ID Nos: 1-4.

12. The recombinant polypeptide of claim 1, wherein the second domain is capable of binding to a carbohydrate antigen or a protein antigen on the surface of a red blood cell.

13. The recombinant polypeptide of claim 12, wherein the carbohydrate antigen is a H antigen, a A antigen, a B antigen, a I antigen, or a Lewis antigen.

14. The recombinant polypeptide of claim 12, wherein the protein antigen is a Rh antigen, a Kell antigen, a Kidd antigen, a Duffy antigen, a Lutheran antigen, a glycophorin A, or a glycophorin B.

15. The recombinant polypeptide of claim 1, wherein the linker is a flexible linker or a rigid linker.

16. The recombinant polypeptide of claim 15, wherein the flexible linker is a glycine-serine linker having the formula (GGGGS)ₓ (SEQ ID NO: 25), a glycine linker comprising at least 5 amino acids, or an immunoglobulin G hinge region.

17. The recombinant polypeptide of claim 15, wherein the a rigid linker is an alpha helical linker having the formula (EAAAK)ₓ (SEQ ID NO: 26), an immunoglobulin domain, or a fibronectin-type domain.

18. The recombinant polypeptide of claim 1, wherein the linker comprises the sequence of SEQ ID NOs: 21, 22, 23 or 24.

19. The recombinant polypeptide of claim 17, wherein the immunoglobulin domain is a constant domain of immunoglobulin A, M, D, E, or G, or the CH1, CH2, CH3, or CL domain of immunoglobulin A, M, D, E, or G.

20. The recombinant polypeptide of claim 17, wherein the immunoglobulin domain has a mutation to abrogate natural dimerization to ensure monomer formation.

21. The recombinant polypeptide of claim 1, wherein the first domain is one or more of SEQ ID NOs: 5-15, 36 or 37 or an antigenic fragment thereof, wherein the linker is one of SEQ ID NOs: 21-24; and wherein the second domain is one of SEQ ID Nos: 1-4.

22. The recombinant polypeptide of claim 1, wherein the first domain is positioned at the N-terminus and the second domain is positioned at the C-terminus of the recombinant polypeptide.

23. The recombinant polypeptide of claim 1, wherein the second domain is positioned at the N-terminus and the first domain is positioned at the C-terminus of the recombinant polypeptide.

24. The recombinant polypeptide of claim 1, further comprising one or more residues positioned at the N-terminus, C-terminus, or both the N-terminus and C-terminus of the recombinant polypeptide, wherein the one or more residues are glycine, alanine or serine or a combination thereof.

25. The recombinant polypeptide of claim 1, further comprising one or more spacer sequences.

26. The recombinant polypeptide of claim 1, wherein the first domain is SEQ ID NO: 14, and the second domain is SEQ ID NO: 1.

27. The recombinant polypeptide of claim 1, wherein the first domain is SEQ ID NO: 15, and the second domain is SEQ ID NO: 2.

28. The recombinant polypeptide of claim 27, wherein the linker is SEQ ID NO: 23.

29. The recombinant polypeptide of claim 1, wherein the first domain is SEQ ID NO: 8, and the second domain is SEQ ID NO: 3.

30. The recombinant polypeptide of claim 29, wherein the linker is SEQ ID NO: 23.

31. The recombinant polypeptide of claim 1, wherein the first domain is SEQ ID NO: 15, and the second domain is SEQ ID NO: 4.

32. The recombinant polypeptide of claim 31, wherein the linker is SEQ ID NO: 22.

33. The recombinant polypeptide of claim 1, further comprising a protein purification affinity tag.

34. The recombinant polypeptide of claim 33, wherein the protein purification affinity tag is a His-tag, a FLAG-tag, a HA-tag, a Strep-tag, a C9-tag, a glutathione S-transferase tag, a maltose-binding protein tag, a T7 tag, a V5 tag, an S tag, a SUMO tag, a TAP tag, a TRX tag, a calmodulin binding peptide, a chitin binding domain, a E2 epitope, a HaloTag, a HSV tag, a HBH tag, a KT3 tag or a myc-tag.

35. A method for detecting one or more anti-coronavirus antibodies in a sample, the method comprising:
a) incubating the sample with a recombinant polypeptide of claim 1, wherein the sample comprises one or more red blood cells;
b) mixing the sample with the recombinant polypeptide, wherein the second domain is capable of binding to the one or more red blood cells in the sample; and
c) observing or determining whether the one or more red blood cells of the sample are agglutinated;
thereby detecting one or more anti-coronavirus antibodies in the sample.

36. The method of claim 35, wherein the sample is whole blood.

37. The method of claim 35, wherein the sample is a serum sample or a plasma sample, and wherein heterologous red blood cells are added to the serum sample or plasma sample.

38. The method of claim 35, wherein the sample is from a subject exposed to or suspected of being exposed to a coronavirus.

39. The method of claim 38, wherein the epitope of a coronavirus is an epitope of a severe acute respiratory syndrome coronavirus 2 (SARS-CoV-2), a severe acute respiratory syndrome (SARS), middle east respiratory syndrome (MERS), a human coronavirus 229E, a human coronavirus NL63, Miniopterus bat coronavirus 1, a Miniopterus bat coronavirus HKU8, porcine epidemic diarrhea virus, a Rhinolophus bat coronavirus HKU2, a Scotophilus bat coronavirus 512, a bovine coronavirus, a human coronavirus OC43, a human coronavirus HKU1, murine coronavirus, a Pipistrellus bat coronavirus HKU5, a Rousettus bat coronavirus HKU9, a Tylonycteris bat coronavirus HKU4, a hedgehog coronavirus 1, an infectious bronchitis virus, a beluga whale coronavirus SW1, an infectious bronchitis virus, a Bulbul coronavirus HKU11, a pangolin coronavirus, a porcine coronavirus HKU15, a WIV1-CoV, a SHC014-CoV, a bat-SL-CoVZC45, a bat-SLCoVZXC21, a SARS-CoVGZ02, a BtKY72, a WIV16, Rs4231, a Rs7327, a Rs9401, a BtRs-BetaCoV/YN2018R, a BtRs-BetaCoV/YN2013, Anlong-112, a Rf2092, a BtRs-BetaCoV/YN2018C, a As6526, Rs4247, a BtRs-BetaCoV/GX2013, a Yunnan2011, a BtRl-BetaCoV/SC2018, a Shannxi2011, a BtRs-BetaCoV/HuB2013, a Bat_CoV_279/2005, a HKU3-12, a HKU3-3, a HKU3-7, a Longquan-140, or a RaTG13.

40. The method of claim 35, further comprising mixing the recombinant polypeptide with a second sample, wherein the second sample is from a subject not exposed to a coronavirus.

41. The method of claim of claim 35, further comprising mixing the recombinant polypeptide with a second sample in the presence of an antibody that is capable of specifically binding to the first domain, wherein the second sample comprises an epitope of a coronavirus.

42. The method of claim 35, wherein the recombinant polypeptide further comprises a label.

43. The method of claim 42, wherein the label is detected by mixing the sample with the recombinant polypeptide in step b) in the presence of an antibody capable of specifically binding to the label thereby detecting agglutination by the binding of the antibody to the label.

44. The method of claim 43, further comprising mixing the sample with the recombinant polypeptide in step b) in the presence of an antibody capable of specifically binding to the sequence specific for an antigen on the surface of the one or more red blood cells of the second domain of the recombinant polypeptide, thereby yielding agglutination.

45. The method of claim 35, wherein the mixing and/or observing is performed in a plate well, on a slide, in a test tube, a gel card, a dry card, by an automated machine, or a microfluidic chip.

46. The method of claim 35, further comprising determining antibody titer in the sample, wherein a serial dilution of the patient whole blood or patient serum or plasma is made prior to incubation with recombinant polypeptide to determine antibody titer.

47. A recombinant polypeptide comprising:
a first domain, wherein the first domain is a moiety that is capable of specifically binding a coronavirus antigen;
a linker; and
a second domain, wherein the second domain is a moiety that is capable of specifically binding an antigen on the surface of a red blood cell.

48. The recombinant polypeptide of claim 47, wherein the coronavirus antigen is a severe acute respiratory syndrome coronavirus 2 (SARS-CoV-2), a severe acute respiratory syndrome (SARS), middle east respiratory syndrome (MERS), a human coronavirus 229E, a human coronavirus NL63, Miniopterus bat coronavirus 1, a Miniopterus bat coronavirus HKU8, porcine epidemic diarrhea virus, a Rhinolophus bat coronavirus HKU2, a Scotophilus bat coronavirus 512, a bovine coronavirus, a human coronavirus OC43, a human coronavirus HKU1, murine coronavirus, a Pipistrellus bat coronavirus HKU5, a Rousettus bat coronavirus HKU9, a Tylonycteris bat coronavirus HKU4, a hedgehog coronavirus 1, an infectious bronchitis virus, a beluga whale coronavirus SW1, an infectious bronchitis virus, a Bulbul coronavirus HKU11, a pangolin coronavirus, a porcine coronavirus HKU15, a WIV1-CoV, a SHC014-CoV, a bat-SL-CoVZC45, a bat-SLCoVZXC21, a SARS-CoVGZ02, a BtKY72, a WIV16, Rs4231, a Rs7327, a Rs9401, a BtRs-BetaCoV/YN2018R, a BtRs-BetaCoV/YN2013, Anlong-112, a Rf2092, a BtRs-BetaCoV/YN2018C, a As6526, Rs4247, a BtRs-BetaCoV/GX2013, a Yunnan2011, a BtRl-BetaCoV/SC2018, a Shannxi2011, a BtRs-BetaCoV/HuB2013, a Bat_CoV_279/2005, a HKU3-12, a HKU3-3, a HKU3-7, a Longquan-140, or a RaTG13 antigen.

49. The recombinant polypeptide of claim 47 or 48, wherein the coronavirus antigen comprises a sequence from a coronavirus spike (S) protein, envelope (E) protein, a membrane (M) protein, a nucleocapsid (N) protein, an antigenic fragment thereof, or a combination thereof.

50. The recombinant polypeptide of claim 49, wherein the coronavirus spike (S) protein comprises a S1 domain, a S2 domain, the N-terminal domain, a receptor-binding domain or the entire S protein ectodomain.

51. The recombinant polypeptide of any of claims 47-50, wherein the first domain is a single chain variable fragment (scFv), a Fab, a camelid antibody, a nanobody, a shark vNAR antibody, adnectins, anticalins, avimers, Fynomers, Kunitz domains, knottins, affibodies, β-hairpin mimetics, designed ankyrin repeat proteins, or a peptide capable of binding to the coronavirus antigen or a portion of the angiotensin converting enzyme-2 (ACE2) protein receptor that binds to the coronavirus spike protein.

52. The recombinant polypeptide of claim 51, wherein the scFv is derived from an antibody capable of specifically binding to a spike protein.

53. The recombinant polypeptide of claim 52, wherein the scFv is derived from CR3022, CR3013, m396, 80R, F26G29, 18F3, 7B11, B38, H4, CA1, CB6, S309, 47D11, 311mab-31B5, 311mab-32D4, 311mab-31B9, H014, 5A6, COV2-2196, COV-2130, COV2-2381, 414-1, P2C-1F11, P2B-2F6, or aP2C-1A3 antibody.

54. The recombinant polypeptide of claim 51, wherein the scFv is derived from an antibody capable of specifically binding to a nucleocapsid protein.

55. The recombinant polypeptide of claim 54, wherein the scFv is derived from S-A5D5, 18F629.1, P140.20B7, P140.19B6, P140.19C7, S-39-2, S-125-2, S-144-3, S-162-2, N-17-3, N-30-12, CR3009, CR3018, N10E4, N1E8, N8E1, N18, MA2.D5, MA2.D7, MA2.E3, A17, mBG17, mBG21, mBG22, mBG57, or mBG67 antibody.

56. The recombinant polypeptide of claim 55, wherein the nanobody is SARS VHH-72 or MERS VHH-55.

57. The recombinant polypeptide of claim 55, wherein the portion of the ACE2 protein receptor is derived from human ACE2 (amino acid 18-615) or human ACE2 (amino acid 18-740) or human ACE2 (amino acid 18-55) or human ACE2 (amino acid 18-88).

58. The recombinant polypeptide of claim 47, wherein the second domain is a single chain variable fragment (scFv), a Fab, a camelid antibody, a nanobody, a shark vNAR antibody, adnectins, anticalins, avimers, Fynomers, Kunitz domains, knottins, affibodies, β-hairpin mimetics, designed ankyrin repeat proteins, or a peptide capable of binding to an antigen on the surface of a red blood cells.

59. The recombinant polypeptide of claim 58, wherein the scFv is derived from an antibody capable of specifically binding to an antigen on the surface of a red blood cells.

60. The recombinant polypeptide of claim 59, wherein the scFv is 10F7, A41, B6, 2E8, 1C3, ABO.B1, ABO.HI1, Rh.D1, Rh.E1, Ery.X1, K.Kpb1, 4G11, or a single domain antibody 1H4.

61. The recombinant polypeptide of claim 62, wherein the second domain is 10 to 100 amino acids long and is derived from an erythrocyte-binding sequence, wherein the erythrocyte-binding sequence is from a Plasmodium protein, wherein the Plasmodium protein is SERA, STEVOR, erythrocyte binding antigen-175, erythrocyte binding antigen-181, erythrocyte binding antigen-140, erythrocyte-binding ligand-1, or PfGARP.

62. The recombinant polypeptide of claim 58, wherein the second domain comprises SEQ ID NOs: 1, 2, 3 or 4.

63. The recombinant polypeptide of claim 58, wherein the second domain is capable of binding to a carbohydrate antigen or a protein antigen on the surface of a red blood cell.

64. The recombinant polypeptide of claim 63, wherein the carbohydrate antigen is a H antigen, a A antigen, a B antigen, a I antigen, or a Lewis antigen.

65. The recombinant polypeptide of claim 63, wherein the protein antigen is a Rh antigen, a Kell antigen, a Kidd antigen, a Duffy antigen, a Lutheran antigen, a glycophorin A, or a glycophorin B.

66. The recombinant polypeptide of any of claims 47-63, wherein the linker is a flexible linker or a rigid linker.

67. The recombinant polypeptide of claim 66, wherein the flexible linker is a glycine-serine linker having the formula (GGGGS)_{X} (SEQ ID NO: 25), a glycine linker comprising at least 5 amino acids, or an immunoglobulin G hinge region.

68. The recombinant polypeptide of claim 66, wherein the rigid linker is an alpha helical linker having the formula (EAAAK)_{X} (SEQ ID NO: 26), an immunoglobulin domain, or a fibronectin-type domain.

69. The recombinant polypeptide of claim 66, wherein the linker comprises the sequence of SEQ ID NOs: 21, 22, 23 or 24.

70. The recombinant polypeptide of claim 68, wherein the immunoglobulin domain is a constant domain of immunoglobulin A, M, D, E, or G, or the CH1, CH2, CH3, or CL domain of immunoglobulin A, M, D, E, or G.

71. The recombinant polypeptide of claim 70, wherein the immunoglobulin domain has a mutation to abrogate natural dimerization to ensure monomer formation.

72. The recombinant polypeptide of any of claims 47-71, wherein the first domain is positioned at the N-terminus and the second domain is positioned at the C-terminus of the recombinant polypeptide.

73. The recombinant polypeptide of any of claims 47-71, wherein the second domain is positioned at the N-terminus and the first domain is positioned at the C-terminus of the recombinant polypeptide.

74. The recombinant polypeptide of any of claims 47-71, further comprising one or more residues positioned at the N-terminus, C-terminus, or both the N-terminus and C-terminus of the recombinant polypeptide, wherein the one or more residues are glycine, alanine or serine or a combination thereof.

75. The recombinant polypeptide of any of claims 47-74, further comprising one or more spacer sequences.

76. The recombinant polypeptide of any of claims 47-75, further comprising a protein purification affinity tag.

77. The recombinant polypeptide of claim 76, wherein the protein purification affinity tag is a His-tag, a FLAG-tag, a HA-tag, a Strep-tag, a C9-tag, a glutathione S-transferase tag, a maltose-binding protein tag, a T7 tag, a V5 tag, an S tag, a SUMO tag, a TAP tag, a TRX tag, a calmodulin binding peptide, a chitin binding domain, a E2 epitope, a HaloTag, a HSV tag, a HBH tag, a KT3 tag or a myc-tag.

78. A method for detecting one or more coronavirus antigens or one or more coronavirus virions in a sample, the method comprising:
a) incubating the sample with a recombinant polypeptide of claim 47, wherein the sample comprises one or more red blood cells;
b) mixing the sample with the recombinant polypeptide, wherein the second domain is capable of binding to the one or more red blood cells in the sample; and
c) observing or determining whether the one or more red blood cells of the sample are agglutinated;
thereby detecting one or more coronavirus antigens or one more coronavirus virions in the sample.

79. The method of claim 78, wherein the sample is whole blood; saliva; viral transport media, wherein the viral transport media is generated from a nasopharyngeal or oropharyngeal swab; nasopharyngeal or oropharyngeal aspirate; respiratory secretions; sputum; bronchalveolar lavage fluid; plasma; or serum.

80. The method of claim 78, further comprising adding autologous or heterologous red blood cells to the sample.

81. The method of claim 78, wherein the sample is from a subject exposed to or suspected of being exposed to a coronavirus.

82. The method of claim 78, wherein the one or more coronavirus antigens is severe acute respiratory syndrome coronavirus 2 (SARS-CoV-2), a severe acute respiratory syndrome (SARS), middle east respiratory syndrome (MERS), a human coronavirus 229E, a human coronavirus NL63, Miniopterus bat coronavirus 1, a Miniopterus bat coronavirus HKU8, porcine epidemic diarrhea virus, a Rhinolophus bat coronavirus HKU2, a Scotophilus bat coronavirus 512, a bovine coronavirus, a human coronavirus OC43, a human coronavirus HKU1, murine coronavirus, a Pipistrellus bat coronavirus HKU5, a Rousettus bat coronavirus HKU9, a Tylonycteris bat coronavirus HKU4, a hedgehog coronavirus 1, an infectious bronchitis virus, a beluga whale coronavirus SW1, an infectious bronchitis virus, a Bulbul coronavirus HKU11, a pangolin coronavirus, a porcine coronavirus HKU15, a WIV1-CoV, a SHC014-CoV, a bat-SL-CoVZC45, a bat-SLCoVZXC21, a SARS-CoVGZ02, a BtKY72, a WIV16, Rs4231, a Rs7327, a Rs9401, a BtRs-BetaCoV/YN2018R, a BtRs-BetaCoV/YN2013, Anlong-112, a Rf2092, a BtRs-BetaCoV/YN2018C, a As6526, Rs4247, a BtRs-BetaCoV/GX2013, a Yunnan2011, a BtRl-BetaCoV/SC2018, a Shannxi2011, a BtRs-BetaCoV/HuB2013, a Bat_CoV_279/2005, a HKU3-12, a HKU3-3, a HKU3-7, a Longquan-140, or a RaTG13 antigen.

83. The method of claim 78, further comprising mixing the recombinant polypeptide with a second sample, wherein the second sample is from a subject not exposed to a coronavirus.

84. The method of claim 78, further comprising mixing the recombinant polypeptide with a second sample in the presence of an antibody that is capable of specifically binding to the first domain, wherein the second sample comprises one or more coronavirus antigens or one more coronavirus virions.

85. The method of claim 78, wherein the recombinant polypeptide further comprises a label.

86. The method of claim 85, wherein the label is detected by mixing the sample with the recombinant polypeptide in step b) in the presence of an antibody capable of specifically binding to the label thereby detecting agglutination by the binding of the antibody to the label.

87. The method of claim 78, wherein the mixing and/or observing is performed in a plate well, on a slide, in a test tube, a gel card, a dry card, automated machine, or a microfluidic chip.


SEQUENCE LISTING (42 sequences)

SEQ ID NO: 1 - protein, 245 aa, Artificial Sequence
  Synthetic construct
QVQLKESGPGLVAPSSQLSITCTVSGFSLSGYSVHWVRQPPGKGLEWLGMIWGGGNTDYKSALKSRLTISKDNSRSQVLLKMNSLQIDDTAIYYCARNYGYSPFVHWGQGTLVTVSAGGGGSGGGGSGGGGSDIVMTQSPSSLAMSVGQKVTMSCKSSQSLLNSDNQKNYLAWYQQKPGQSPKLLVYFASSRESGVSDRFIGSGSGTDFTLTIGSVQSEDLAYYFCQQLYRTPFTFGSGTKLEIK

SEQ ID NO: 2 - protein, 246 aa, Artificial Sequence
  synthetic construct
QVKLVQSGPELVKPGASVKMSCKASGYTFTNYVMHWVKQQPGQVLEWIGYINPYNDDTKYNEKFKGKATLASDKSSNTAYMELSSLPSEDSAVYYCASGGYYTMDYWGQGTSVTVSSGGGGSGGGGSGGGGSDIVLTQSPLSLPVSLGDQASISCRSSQSLVHSKGYTYLHWYLQKPGQSPKLLIYKVSRRFSGVPDRFNGSGSGTDFTLKISRVEAEDLGVYFCSQSTHVPYTFGGGTKLELKRA

SEQ ID NO: 3 - protein, 246 aa, Artificial Sequence
  Synthetic construct
QVKLQQSGAELVKPGASVKLSCKASGYTFNSYFMHWMKQRPVQGLEWIGMIRPNGGTTDYNEKFKNKATLTVDKSSNTAYMQLNSLTSGDSAVYYCARWEGSYYALDYWGQGTTVTVSSGGGGSGGGGSSGGGGSSDIELTQSPAIMSATLGEKVTMTCRASSNVKYMYWYQQKSGASPKLWIYYTSNLASGVPGRFSGSGSGTSYSLTISSVEAEDAATYYCQQFTSSPYTFGGGTKLEIKRAAA

SEQ ID NO: 4 - protein, 122 aa, Artificial Sequence
  Synthetic construct
QVQLQESGGGSVQAGGSLRLSCVASGYTDSTYCVGWFRQAPGKEREGVARINTISGRPWYADSVKGRFTISQDNSKNTVFLQMNSLKPEDTAIYYCTLTTANSRGFCSGGYNYKGQGQVTVS

SEQ ID NO: 5 - protein, 1203 aa, Artificial Sequence
  Synthetic construct
VSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 6 - protein, 1203 aa, Artificial Sequence
  Synthetic construct
VSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPAAAASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 7 - protein, 419 aa, Artificial Sequence
  Synthetic construct
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKPRQKRTATKAYNVTQAFGRRGPEQTQGNFGDQELIRQGTDYKHWPQIAQFAPSASAFFGMSRIGMEVTPSGTWLTYTGAIKLDDKDPNFKDQVILLNKHIDAYKTFPPTEPKKDKKKKADETQALPQRQKKQQTVTLLPAADLDDFSKQLQQSMSSADSTQA

SEQ ID NO: 8 - protein, 319 aa, Artificial Sequence
  Synthetic construct
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKGSGSTFPPTEPKKDKKKKADETQALPQRQKKQQTVTLLPAADLDDFSKQLQQSMSSADSTQA

SEQ ID NO: 9 - protein, 75 aa, Artificial Sequence
  Synthetic construct
MYSFVSEETGTLIVNSVLLFLAFVVFLLVTLAILTALRLCAYCCNIVNVSLVKPSFYVYSRVKNLNSSRVPDLLV

SEQ ID NO: 10 - protein, 222 aa, Artificial Sequence
  Synthetic construct
MADSNGTITVEELKKLLEQWNLVIGFLFLTWICLLQFAYANRNRFLYIIKLIFLWLLWPVTLACFVLAAVYRINWITGGIAIAMACLVGLMWLSYFIASFRLFARTRSMWSFNPETNILLNVPLHGTILTRPLLESELVIGAVILRGHLRIAGHHLGRCDIKDLPKEITVATSRTLSYYKLGASQRVAGDSGFAAYSRYRIGNYKLNTDHSSSSDNIALLVQ

SEQ ID NO: 11 - protein, 671 aa, Artificial Sequence
  Synthetic construct
VSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSP

SEQ ID NO: 12 - protein, 528 aa, Artificial Sequence
  Synthetic construct
SVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 13 - protein, 528 aa, Artificial Sequence
  Synthetic construct
SVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 14 - protein, 195 aa, Artificial Sequence
  Synthetic construct
PNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATV

SEQ ID NO: 15 - protein, 231 aa, Artificial Sequence
  Synthetic construct
RVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGT

SEQ ID NO: 16 - protein, 464 aa, Artificial Sequence
  Synthetic construct
PNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVTNLVKNKSVNSNSNGLTGTGSGSGQVQLKESGPGLVAPSQSLSITCTVSGFSLSGYSVHWVRQPPGKGLEWLGMIWGGGNTDYKSALKSRLTISKDNSRSQVLLKMNSLQIDDTAIYYCARNYGYSPFVHWGQGTLVTVSAGGGGSGGGGSGGGGSDIVMTQSPSSLAMSVGQKVTMSCKSSQSLLNSDNQKNYLAWYQQKPGQSPKLLVYFASSRESGVSDRFIGSGSGTDFTLTIGSVQSEDLAYYFCQQLYRTPFTFGSGTKLEIK

SEQ ID NO: 17 - protein, 590 aa, Artificial Sequence
  Synthetic construct
QVKLVQSGPELVKPGASVKMSCKASGYTFTNYVMHWVKQQPGQVLEWIGYINPYNDDTKYNEKFKGKATLASDKSSNTAYMELSSLPSEDSAVYYCASGGYYTMDYWGQGTSVTVSSGGGGSGGGGSGGGGSDIVLTQSPLSLPVSLGDQASISCRSSQSLVHSKGYTYLHWYLQKPGQSPKLLIYKVSRRFSGVPDRFNGSGSGTDFTLKISRVEAEDLGVYFCSQSTHVPYTFGGGTKLELKRAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSGSGGGSGGSGGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGT

SEQ ID NO: 18 - protein, 678 aa, Artificial Sequence
  Synthetic construct
QVKLQQSGAELVKPGASVKLSCKASGYTFNSYFMHWMKQRPVQGLEWIGMIRPNGGTTDYNEKFKNKATLTVDKSSNTAYMQLNSLTSGDSAVYYCARWEGSYYALDYWGQGTTVTVSSGGGGSGGGGSSGGGGSSDIELTQSPAIMSATLGEKVTMTCRASSNVKYMYWYQQKSGASPKLWIYYTSNLASGVPGRFSGSGSGTSYSLTISSVEAEDAATYYCQQFTSSPYTFGGGTKLEIKRAAAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSGSGGGSGGSGGMSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFYAEGSRGGSQASSRSSSRSRNSSRNSTPGSSRGTSPARMAGNGGDAALALLLLDRLNQLESKMSGKGQQQQGQTVTKKSAAEASKKGSGSTFPPTEPKKDKKKKADETQALPQRQKKQQTVTLLPAADLDDFSKQLQQSMSSADSTQA

SEQ ID NO: 19 - protein, 1028 aa, Artificial Sequence
  Synthetic construct
QVQLKESGPGLVAPSSQLSITCTVSGFSLSGYSVHWVRQPPGKGLEWLGMIWGGGNTDYKSALKSRLTISKDNSRSQVLLKMNSLQIDDTAIYYCARNYGYSPFVHWGQGTLVTVSAGGGGSGGGGSGGGGSDIVMTQSPSSLAMSVGQKVTMSCKSSQSLLNSDNQKNYLAWYQQKPGQSPKLLVYFASSRESGVSDRFIGSGSGTDFTLTIGSVQSEDLAYYFCQQLYRTPFTFGSGTKLEIKAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDGTSGGSGGSGGVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSP

SEQ ID NO: 20 - protein, 761 aa, Artificial Sequence
  Synthetic construct
QVQLQESGGGSVQAGGSLRLSCVASGYTDSTYCVGWFRQAPGKEREGVARINTISGRPWYADSVKGRFTISQDNSKNTVFLQMNSLKPEDTAIYYCTLTTANSRGFCSGGYNYKGQGQVTVSAKTTPPSVYPLAPGSAAQTNSMVTLGCLVKGYFPEPVTVTWNSGSLSSGVHTFPAVLQSDLYTLSSSVTVPSSTWPSETVTCNVAHPASSKVDKKIVPRDGTSGGSGGSGGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 21 - protein, 20 aa, Artificial Sequence
  Synthetic construct
GGGGSGGGGSGGGGSGGGGS

SEQ ID NO: 22 - protein, 104 aa, Artificial Sequence
  Synthetic construct
AKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDGTS

SEQ ID NO: 23 - protein, 105 aa, Artificial Sequence
  Synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSGSG

SEQ ID NO: 24 - protein, 24 aa, Artificial Sequence
  Synthetic construct
TNLVKNKSVNSNSNGLTGTGSGSG

SEQ ID NO: 25 - protein, 100 aa, Artificial Sequence
  Synthetic construct
  misc_feature (6)..(101): can be present or absent
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 26 - protein, 100 aa, Artificial Sequence
  Synthetic construct
  misc_feature (6)..(101): can be present or absent
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 27 - protein, 19 aa, Artificial Sequence
  Synthetic construct
MDWIWRILFLVGAATGAHS

SEQ ID NO: 28 - protein, 245 aa, Artificial Sequence
  Synthetic construct
QMQLVQSGTEVKKPGESLKISCKGSGYGFITYWIGWVRQMPGKGLEWMGIIYPGDSETRYSPSFQGQVTISADKSINTAYLQWSSLKASDTAIYYCAGGSGISTPMDVWGQGTTVTVGGGGSGGGGSGGGGSDIQLTQSPDSLAVSLGERATINCKSSQSVLYSSINKNYLAWYQQKPGQPPKLLIYWASTRESGVPDRFSGSGSGTDFTLTISSLQAEDVAVYYCQQYYSTPYTFGQGTKVEIK

SEQ ID NO: 29 - protein, 244 aa, Artificial Sequence
  Synthetic construct
EVQLVESGGGLVQPGGSLRLSCAASGFTVSSNYMSWVRQAPGKGLEWVSVIYSGGSTFYADSVKGRFTISRDNSMNTLFLQMNSLRAEDTAVYYCARVLPMYGDYLDYWGQGTLVTVSSGGGGSGGGGSGGGGSDIVMTQSPSSLSASVGDRVTITCRASQSISRYLNWYQQKPGKAPKLLIYAASSLQSGVPSRFSGSGSGTDFTLTISSLQPEDFATYYCQQSYSTPPEYTFGQGTKLEIKR

SEQ ID NO: 30 - protein, 598 aa, Artificial Sequence
  Synthetic construct
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 31 - protein, 720 aa, Artificial Sequence
  Synthetic construct
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKVSDIIPRTEVEKAIRMSRSRINDAFRLNDNSLEFLGIQPTLGPPNQPPVS

SEQ ID NO: 32 - protein, 238 aa, Artificial Sequence
  Synthetic construct
EVQLVESGGGVVQPGRSLRLSCAASGFTFSDYPMNWVRQAPGKGLEWVSSISGSGGSTYYADSVKGRFTISRDNSKNTLYLQMNLRAEDTAVYYCAKGLFMVTTYAFDYWGQGTLVTVGGGGSGGGGSGGGGSELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 33 - protein, 236 aa, Artificial Sequence
  Synthetic construct
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKFNPFTSFDYWGQGTLVTVGGGGSGGGGSGGGGSELTQSPSSLSASVGDRVTITCRASQSISSYLNWYQQKPGKAPKLLIYAASSLQSGVPSRFSGSGSGTDFTLTISSLQPEDFATYYCQQSYSTPPTFGQGTKVEIK

SEQ ID NO: 34 - protein, 254 aa, Artificial Sequence
  Synthetic construct
SYVLTQPPSVSVAPGKTARIPCGGNNIGSKSVHWYQQKPGQAPVLVIYYDSDRPSGIPERFSGSNSGNTATLTISRVEAGDEADYYCQVWDRSSDLVVFGGGTKLTVLSGGSTITSYNVYYTKLSSSGTQVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGYWGSGYHYYGMDVWGQGTTVTVSSAS

SEQ ID NO: 35 - protein, 479 aa, Artificial Sequence
  Synthetic construct
MGWSCIILFLVATATGVHSQVKLQQSGAELVKPGASVKLSCKASGYTFNSYFMHWMKQRPVQGLEWIGMIRPNGGTTDYNEKFKNKATLTVDKSSNTAYMQLNSLTSGDSAVYYCARWEGSYYALDYWGQGTTVTVSSGGGGSGGGGSSGGGGSSDIELTQSPAIMSATLGEKVTMTCRASSNVKYMYWYQQKSGASPKLWIYYTSNLASGVPGRFSGSGSGTSYSLTISSVEAEDAATYYCQQFTSSPYTFGGGTKLEIKRAAASSTGSGGSGNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKHHHHHH

SEQ ID NO: 36 - protein, 199 aa, Artificial Sequence
  Synthetic construct
NITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKK

SEQ ID NO: 37 - protein, 199 aa, Artificial Sequence
  Synthetic construct
NITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKK

SEQ ID NO: 38 - protein, 488 aa, Artificial Sequence
  Synthetic construct
MDWIWRILFLVGAATGAHSPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGVIAWNSNNLDSKVGGNYNYLYRLFRKSNLKPFERDISTEIYQAGSTPCNGVEGFNCYFPLQSYGFQPTNGVGYQYRVVVLSFELLHAPATVTNLVKNKSVNSNSNGLTGTGSGSGQVQLKESGPGLVAPSQSLSITCTVSGFSLSGYSVHWVRQPPGKGLEWLGMIWGGGNTDYKSALKSRLTISKDNSRSQVLLKMNSLQIDDTAIYYCARNYGYSPFVHWGQGTLVTVSAGGGGWSGGGGSGGGGSDIVMTQSPSSLAMSVGQKVTMSCKSSQSLLNSDNQKNYLAWYQQKPGQSPKLLVYFASSRESGVSDRFIGSGSGTDFTLTIGSVQSEDLAYYFCQQLYRTPFTFGSGTKLEIKHHHHHH

SEQ ID NO: 39 - protein, 601 aa, Artificial Sequence
  Synthetic construct
MQVKLVQSGPELVKPGASVKMSCKASGYTFTNYVMHWVKQQPGQVLEWIGYINPYNDDTKYNEKFKGKATLASDKSSNTAYMELSSLPSEDSAVYYCASGGYYTMDYWGQGTSVTVSSGGGGSGGGGSGGGGSDIVLTQSPLSLPVSLGDQASISCRSSQSLVHSKGTYYLHWYLQKPGQSPKLLIYKVSRRFSGVPDRFNGSGSGTDFTLKISRVEAEDLGVYFCSQSTHVPYTFGGGTKELKRAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSGSGGGSGGSGGRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGGSGGHHHHHH

SEQ ID NO: 40 - protein, 243 aa, Artificial Sequence
  Synthetic construct
EVQLVESGGGLVQPGGSLKLSCAASGFTFSNYGMSWVRQTPDKRLELVATINRNGGSTYYLDSVKVRFTISRDNAKSTLFLQLSSLKSDDTAMYYCARIYDFDEDYFDVWGAGTTVTVSSGGGGSGGGGSGGGGSQIVLTQSPAIMSASLGERVTMTCTASSSVSSSYLHWYQQKPGSSPKLWIYSTSNLASGVPARFSGSGSGTSYSLTISSMEAEDAATYYCLQYHRSPWTFGGGTKLEIK

SEQ ID NO: 41 - protein, 19 aa, Artificial Sequence
  Synthetic construct
MGWSCIILFLVATATGVHS

SEQ ID NO: 42 - protein, 338 aa, Artificial Sequence
  Synthetic construct
QVQLQESGGGSVQAGGSLRLSCVASGYTDSTYCVGWFRQAPGKEREGVARINTISGRPWYADSVKGRFTISQDNSKNTVFLQMNSLKPEDTAIYYCTLTTANSRGFCSGGYNYKGQGTQVTVSSASTGSGGSGNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKHHHHHH